SEQUENCE LISTING (109 sequences)

SEQ ID NO: 1 - protein, 122 aa, Artificial Sequence
  Description of Artificial Sequence Synthetic polypeptide
EVQLVESGGGLVQPGGSLRLSCAASGFTFSNYWISWVRQAPGKGLEWVAEIRSESDASATHYAEAVKGRFTISRDNAKNSLYLQMNSLRAEDTAVYYCLAYFDYGLAIQNYWGQGTLVTVSS

SEQ ID NO: 2 - protein, 107 aa, Artificial Sequence
  Description of Artificial Sequence Synthetic polypeptide
EIVLTQSPATLSLSPGERATLSCKASKRVTTYVSWYQQKPGQAPRLLIYGASNRYLGIPARFSGSGSGTDFTLTISSLEPEDFAVYYCSQSYNYPYTFGQGTKLEIK

SEQ ID NO: 3 - protein, 10 aa, Artificial Sequence
  Description of Artificial Sequence Synthetic peptide
GFTFSNYWIS

SEQ ID NO: 4 - protein, 19 aa, Artificial Sequence
  Description of Artificial Sequence Synthetic peptide
EIRSESDASATHYAEAVKG

SEQ ID NO: 5 - protein, 11 aa, Artificial Sequence
  Description of Artificial Sequence Synthetic peptide
YFDYGLAIQNY

SEQ ID NO: 6 - protein, 11 aa, Artificial Sequence
  Description of Artificial Sequence Synthetic peptide
KASKRVTTYVS

SEQ ID NO: 7 - protein, 7 aa, Artificial Sequence
  Description of Artificial Sequence Synthetic peptide
GASNRYL

SEQ ID NO: 8 - protein, 9 aa, Artificial Sequence
  Description of Artificial Sequence Synthetic peptide
SQSYNYPYT

SEQ ID NO: 9 - DNA, 366 bp, Artificial Sequence
  Description of Artificial Sequence Synthetic polynucleotide
gaagttcagctggttgaatccggtggtggtctggttcagccaggtggttccctgcgtctgtcctgcgctgcttccggtttcaccttctccaactactggatctcctgggttcgtcaggctcctggtaaaggtctggaatgggttgctgaaatccgttccgaatccgacgcgtccgctacccattacgctgaagctgttaaaggtcgtttcaccatctcccgtgacaacgctaagaactccctgtacctgcagatgaactccctgcgtgctgaagacaccgctgtttactactgcctggcttactttgactacggtctggctatccagaactactggggtcagggtaccctggttaccgtttcctcc

SEQ ID NO: 10 - DNA, 321 bp, Artificial Sequence
  Description of Artificial Sequence Synthetic polynucleotide
gaaatcgttctgacccagtccccggctaccctgtccctgtccccaggtgaacgtgctaccctgtcctgcaaagcttccaaacgggttaccacctacgtttcctggtaccagcagaaacccggtcaggctcctcgtctgctgatctacggtgcttccaaccgttacctcggtatcccagctcgtttctccggttccggttccggtaccgacttcaccctgaccatctcctccctggaacccgaagacttcgctgtttactactgcagtcagtcctacaactacccctacaccttcggtcagggtaccaaactggaaatcaaa

SEQ ID NO: 11 - protein, 448 aa, Artificial Sequence
  Description of Artificial Sequence Synthetic polypeptide
EVQLVESGGGLVQPGGSLRLSCAASGFTFSNYWISWVRQAPGKGLEWVAEIRSESDASATHYAEAVKGRFTISRDNAKNSLYLQMNSLRAEDTAVYYCLAYFDYGLAIQNYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 12 - protein, 214 aa, Artificial Sequence
  Description of Artificial Sequence Synthetic polypeptide
EIVLTQSPATLSLSPGERATLSCKASKRVTTYVSWYQQKPGQAPRLLIYGASNRYLGIPARFSGSGSGTDFTLTISSLEPEDFAVYYCSQSYNYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - DNA, 1347 bp, Artificial Sequence
  Description of Artificial Sequence Synthetic polynucleotide
gaagttcagctggttgaatccggtggtggtctggttcagccaggtggttccctgcgtctgtcctgcgctgcttccggtttcaccttctccaactactggatctcctgggttcgtcaggctcctggtaaaggtctggaatgggttgctgaaatccgttccgaatccgacgcgtccgctacccattacgctgaagctgttaaaggtcgtttcaccatctcccgtgacaacgctaagaactccctgtacctgcagatgaactccctgcgtgctgaagacaccgctgtttactactgcctggcttactttgactacggtctggctatccagaactactggggtcagggtaccctggttaccgtttcctccgcctccaccaagggcccatctgtcttcccactggccccatgctcccgcagcacctccgagagcacagccgccctgggctgcctggtcaaggactacttcccagaacctgtgaccgtgtcctggaactctggcgctctgaccagcggcgtgcacaccttcccagctgtcctgcagtcctcaggtctctactccctcagcagcgtggtgaccgtgccatccagcaacttcggcacccagacctacacctgcaacgtagatcacaagccaagcaacaccaaggtcgacaagaccgtggagagaaagtgttgtgtggagtgtccaccttgtccagcccctccagtggccggaccatccgtgttcctgttccctccaaagccaaaggacaccctgatgatctccagaaccccagaggtgacctgtgtggtggtggacgtgtcccacgaggacccagaggtgcagttcaactggtatgtggacggagtggaggtgcacaacgccaagaccaagccaagagaggagcagttcaactccaccttcagagtggtgagcgtgctgaccgtggtgcaccaggactggctgaacggaaaggagtataagtgtaaggtgtccaacaagggactgccatccagcatcgagaagaccatctccaagaccaagggacagccaagagagccacaggtgtataccctgcccccatccagagaggagatgaccaagaaccaggtgtccctgacctgtctggtgaagggattctatccatccgacatcgccgtggagtgggagtccaacggacagccagagaacaactataagaccacccctccaatgctggactccgacggatccttcttcctgtattccaagctgaccgtggacaagtccagatggcagcagggaaacgtgttctcttgttccgtgatgcacgaggccctgcacaaccactatacccagaagagcctgtccctgtctccaggaaagtaa

SEQ ID NO: 14 - DNA, 645 bp, Artificial Sequence
  Description of Artificial Sequence Synthetic polynucleotide
gaaatcgttctgacccagtccccggctaccctgtccctgtccccaggtgaacgtgctaccctgtcctgcaaagcttccaaacgggttaccacctacgtttcctggtaccagcagaaacccggtcaggctcctcgtctgctgatctacggtgcttccaaccgttacctcggtatcccagctcgtttctccggttccggttccggtaccgacttcaccctgaccatctcctccctggaacccgaagacttcgctgtttactactgcagtcagtcctacaactacccctacaccttcggtcagggtaccaaactggaaatcaaacgcactgtggctgcaccatctgtcttcatcttccctccatctgatgagcagttgaaatccggaactgcctctgttgtgtgcctgctgaataacttctatccgcgcgaggccaaagtacagtggaaggtggataacgccctccaatccggtaactcccaggagagtgtcacagagcaggacagcaaggacagcacctacagcctcagcagcaccctgaccctgagcaaagcagactacgagaaacacaaagtctacgcctgcgaagtcacccatcagggcctgagttctccagtcacaaagagcttcaaccgcggtgagtgctaa

SEQ ID NO: 15 - protein, 37 aa, Homo sapiens
  misc_feature: C-term amidated
ACDTATCVTHRLAGLLSRSGGVVKNNFVPTNVGSKAF

SEQ ID NO: 16 - protein, 30 aa, Homo sapiens
  misc_feature: C-term amidated
VTHRLAGLLSRSGGVVKNNFVPTNVGSKAF

SEQ ID NO: 17 - protein, 19 aa, Homo sapiens
  misc_feature: C-term amidated
SGGVVKNNFVPTNVGSKAF

SEQ ID NO: 18 - protein, 19 aa, Homo sapiens
  misc_feature: C-term amidated
SGGVVKNNFVATNVGSKAF

SEQ ID NO: 19 - protein, 19 aa, Homo sapiens
  misc_feature: C-term amidated
SGGVVKNNFVPTNVGSAAF

SEQ ID NO: 20 - protein, 19 aa, Homo sapiens
  misc_feature: C-term amidated
SGGVVKNNFVPTNVGSEAF

SEQ ID NO: 21 - protein, 19 aa, Homo sapiens
  misc_feature: C-term amidated
SGGVVKNNFVPTNVGSMAF

SEQ ID NO: 22 - protein, 19 aa, Homo sapiens
  misc_feature: C-term amidated
SGGVVKNNFVPTNVGSQAF

SEQ ID NO: 23 - protein, 19 aa, Homo sapiens
  misc_feature: C-term amidated
SGGVVKNNFVPTNVGSKAA

SEQ ID NO: 24 - protein, 14 aa, Homo sapiens
  misc_feature: C-term amidated
NNFVPTNVGSKAFA

SEQ ID NO: 25 - protein, 13 aa, Homo sapiens
  misc_feature: C-term amidated
NNFVPTNVGSKAF

SEQ ID NO: 26 - protein, 13 aa, Homo sapiens
  misc_feature: C-term amidated
NNAVPTNVGSKAF

SEQ ID NO: 27 - protein, 13 aa, Homo sapiens
  misc_feature: C-term amidated
NNFAPTNVGSKAF

SEQ ID NO: 28 - protein, 13 aa, Homo sapiens
  misc_feature: C-term amidated
NNFVATNVGSKAF

SEQ ID NO: 29 - protein, 13 aa, Homo sapiens
  misc_feature: C-term amidated
NNFVPANVGSKAF

SEQ ID NO: 30 - protein, 13 aa, Homo sapiens
  misc_feature: C-term amidated
NNFVPTAVGSKAF

SEQ ID NO: 31 - protein, 13 aa, Homo sapiens
  misc_feature: C-term amidated
NNFVPTNAGSKAF

SEQ ID NO: 32 - protein, 13 aa, Homo sapiens
  misc_feature: C-term amidated
NNFVPTNVASKAF

SEQ ID NO: 33 - protein, 13 aa, Homo sapiens
  misc_feature: C-term amidated
NNFVPTNVGAKAF

SEQ ID NO: 34 - protein, 13 aa, Homo sapiens
  misc_feature: C-term amidated
NNFVPTNVGSKAA

SEQ ID NO: 35 - protein, 12 aa, Homo sapiens
  misc_feature: C-term amidated
NFVPTNVGSKAF

SEQ ID NO: 36 - protein, 19 aa, Homo sapiens
SGGVVKNNFVPTNVGSKAF

SEQ ID NO: 37 - protein, 18 aa, Homo sapiens
SGGVVKNNFVPTNVGSKA

SEQ ID NO: 38 - protein, 36 aa, Homo sapiens
ACDTATCVTHRLAGLLSRSGGVVKNNFVPTNVGSKA

SEQ ID NO: 39 - protein, 19 aa, Homo sapiens
ACDTATCVTHRLAGLLSRS

SEQ ID NO: 40 - protein, 13 aa, Homo sapiens
ACDTATCVTHRLA

SEQ ID NO: 41 - protein, 37 aa, Rattus sp.
  misc_feature: C-term amidated
SCNTATCVTHRLAGLLSRSGGVVKDNFVPTNVGSEAF

SEQ ID NO: 42 - protein, 19 aa, Rattus sp.
  misc_feature: C-term amidated
SGGVVKDNFVPTNVGSEAF

SEQ ID NO: 43 - protein, 37 aa, Homo sapiens
  misc_feature: C-term amidated
ACNTATCVTHRLAGLLSRSGGMVKSNFVPTNVGSKAF

SEQ ID NO: 44 - protein, 37 aa, Rattus sp.
  misc_feature: C-term amidated
SCNTATCVTHRLAGLLSRSGGVVKDNFVPTNVGSKAF

SEQ ID NO: 45 - protein, 32 aa, Homo sapiens
  misc_feature: C-term amidated
CGNLSTCMLGTYTQDFNKFHTFPQTAIGVGAP

SEQ ID NO: 46 - protein, 37 aa, Homo sapiens [sequence omitted]

SEQ ID NO: 47 - protein, 52 aa, Homo sapiens
  misc_feature: C-term amidated
YRQSMNNFQGLRSFGCRFGTCTVQKLAHQIYQFTDKDKDNVAPRSKISPQGY

SEQ ID NO: 48 - protein, 4 aa, Artificial Sequence
  Description of Artificial Sequence Synthetic peptide
ELLG

SEQ ID NO: 49 - protein, 4 aa, Artificial Sequence
  Description of Artificial Sequence Synthetic peptide
ELLG

SEQ ID NO: 50 - protein, 3 aa, Artificial Sequence
  Description of Artificial Sequence Synthetic peptide
PVA

SEQ ID NO: 51 - protein, 3 aa, Artificial Sequence
  Description of Artificial Sequence Synthetic peptide
ELL

SEQ ID NO: 52 - protein, 4 aa, Artificial Sequence
  Description of Artificial Sequence Synthetic peptide
EFLG

SEQ ID NO: 53 - protein, 11 aa, Artificial Sequence
  Description of Artificial Sequence Synthetic peptide
  MOD_RES (5)..(5): Arg, Trp, Gly, Leu or Asn
  MOD_RES (7)..(7): Thr, Ala, Asp, Gly, Arg, Ser, Trp or Val
KASKXVXTYVS

SEQ ID NO: 54 - protein, 7 aa, Artificial Sequence
  Description of Artificial Sequence Synthetic peptide
  MOD_RES (1)..(1): Gly or Ala
  MOD_RES (2)..(2): Ala or His
  MOD_RES (7)..(7): Leu, Thr, Ile or Ser
XXSNRYX

SEQ ID NO: 55 - protein, 19 aa, Artificial Sequence
  Description of Artificial Sequence Synthetic peptide
  MOD_RES (5)..(5): Glu, Arg, Lys, Gln or Asn
  MOD_RES (8)..(8): Ala, Gly, Asn, Glu, His, Ser, Leu, Arg, Cys, Phe, Tyr, Val, Asp or Pro
  MOD_RES (9)..(9): Ser, Gly, Thr, Tyr, Cys, Glu, Leu, Ala, Pro, Ile, Asn, Arg, Val, Asp or Met
  MOD_RES (12)..(12): His or Phe
  MOD_RES (15)..(15): Glu or Asp
EIRSXSDXXATXYAXAVKG

SEQ ID NO: 56 - protein, 6 aa, Artificial Sequence
  Description of Artificial Sequence Synthetic 6xHis tag
HHHHHH

SEQ ID NO: 57 - protein, 15 aa, Artificial Sequence
  Description of Artificial Sequence Synthetic peptide
GGGGSGGGGSGGGGS

SEQ ID NO: 58 - protein, 107 aa, Artificial Sequence
  Description of Artificial Sequence Synthetic polypeptide
DIQMTQSPSSLSASVGDRVTITCRASQDIDNYLNWYQQKPGKAPKLLIYYTSEYHSGVPSRFSGSGSGTDFTFTISSLQPEDIATYYCQQGDALPPTFGQGTKLEIK

SEQ ID NO: 59 - protein, 119 aa, Artificial Sequence
  Description of Artificial Sequence Synthetic polypeptide
QVQLVQSGAEVKKPGASVKVSCKASGYTFGNYWMQWVRQAPGQGLEWMGAIYEGTGDTRYIQKFAGRVTMTRDTSTSTVYMELSSLRSEDTAVYYCARLSDYVSGFSYWGQGTLVTVSS

SEQ ID NO: 60 - protein, 107 aa, Artificial Sequence; identical to SEQ ID NO: 58
  Description of Artificial Sequence Synthetic polypeptide

SEQ ID NO: 61 - protein, 119 aa, Artificial Sequence
  Description of Artificial Sequence Synthetic polypeptide
QVQLVQSGAEVKKPGASVKVSCKASGYTFGNYWMQWVRQAPGQGLEWMGAIYEGTGKTVYIQKFAGRVTMTRDTSTSTVYMELSSLRSEDTAVYYCARLSDYVSGFSYWGQGTLVTVSS

SEQ ID NO: 62 - protein, 107 aa, Artificial Sequence
  Description of Artificial Sequence Synthetic polypeptide
DIQMTQSPSSLSASVGDRVTITCRASKDISKYLNWYQQKPGKAPKLLIYYTSGYHSGVPSRFSGSGSGTDFTLTISSLQPEDFATYYCQQGDALPPTFGGGTKVEIK

SEQ ID NO: 63 - protein, 119 aa, Artificial Sequence
  Description of Artificial Sequence Synthetic polypeptide
QVQLVQSGAEVKKPGSSVKVSCKASGYTFGNYWMQWVRQAPGQGLEWMGAIYEGTGKTVYIQKFADRVTITADKSTSTAYMELSSLRSEDTAVYYCARLSDYVSGFGYWGQGTTVTVSS

SEQ ID NO: 64 - protein, 107 aa, Artificial Sequence
  Description of Artificial Sequence Synthetic polypeptide
DIQMTQSPSSLSASVGDRVTITCRASRPIDKYLNWYQQKPGKAPKLLIYYTSEYHSGVPSRFSGSGSGTDFTFTISSLQPEDIATYYCQQGDALPPTFGQGTKLEIK

SEQ ID NO: 65 - protein, 119 aa, Artificial Sequence
  Description of Artificial Sequence Synthetic polypeptide
QVQLVQSGAEVKKPGASVKVSCKASGYTFGNYWMQWVRQAPGQGLEWMGAIYEGTGKTVYIQKFAGRVTMTRDTSTSTVYMELSSLRSEDTAVYYCARLSDYVSGFGYWGQGTLVTVSS

SEQ ID NO: 66 - protein, 107 aa, Artificial Sequence
  Description of Artificial Sequence Synthetic polypeptide
DIQMTQSPSSLSASVGDRVTITCRASQDIDKYLNWYQQKPGKAPKLLIYYTSGYHSGVPSRFSGSGSGTDFTLTISSLQPEDFATYYCQQGDALPPTFGGGTKVEIK

SEQ ID NO: 67 - protein, 119 aa, Artificial Sequence
  Description of Artificial Sequence Synthetic polypeptide
QVQLVQSGAEVKKPGSSVKVSCKASGYTFGNYWMQWVRQAPGQGLEWMGAIYEGTGKTVYIQKFAGRVTITADKSTSTAYMELSSLRSEDTAVYYCARLSDYVSGFGYWGQGTTVTVSS

SEQ ID NO: 68 - protein, 113 aa, Artificial Sequence
  Description of Artificial Sequence Synthetic polypeptide
QVLTQSPSSLSASVGDRVTINCQASQSVYHNTYLAWYQQKPGKVPKQLIYDASTLASGVPSRFSGSGSGTDFTLTISSLQPEDVATYYCLGSYDCTNGDCFVFGGGTKVEIKR

SEQ ID NO: 69 - protein, 111 aa, Artificial Sequence
  Description of Artificial Sequence Synthetic polypeptide
EVQLVESGGGLVQPGGSLRLSCAVSGIDLSGYYMNWVRQAPGKGLEWVGVIGINGATYYASWAKGRFTISRDNSKTTVYLQMNSLRAEDTAVYFCARGDIWGQGTLVTVSS

SEQ ID NO: 70 - protein, 113 aa, Artificial Sequence
  Description of Artificial Sequence Synthetic polypeptide
QVLTQSPSSLSASVGDRVTINCQASQSVYDNNYLAWYQQKPGKVPKQLIYSTSTLASGVPSRFSGSGSGTDFTLTISSLQPEDVATYYCLGSYDCSSGDCFVFGGGTKVEIKR

SEQ ID NO: 71 - protein, 111 aa, Artificial Sequence
  Description of Artificial Sequence Synthetic polypeptide
EVQLVESGGGLVQPGGSLRLSCAVSGLDLSSYYMQWVRQAPGKGLEWVGVIGINDNTYYASWAKGRFTISRDNSKTTVYLQMNSLRAEDTAVYFCARGDIWGQGTLVTVSS

SEQ ID NO: 72 - protein, 113 aa, Artificial Sequence; identical to SEQ ID NO: 70
  Description of Artificial Sequence Synthetic polypeptide

SEQ ID NO: 73 - protein, 111 aa, Artificial Sequence; identical to SEQ ID NO: 71
  Description of Artificial Sequence Synthetic polypeptide

SEQ ID NO: 74 - protein, 113 aa, Artificial Sequence
  Description of Artificial Sequence Synthetic polypeptide
QVLTQTPSPVSAAVGSTVTINCQASQSVYHNTYLAWYQQKPGQPPKQLIYDASTLASGVPSRFSGSGSGTQFTLTISGVQCNDAAAYYCLGSYDCTNGDCFVFGGGTEVVVKR

SEQ ID NO: 75 - protein, 109 aa, Artificial Sequence
  Description of Artificial Sequence Synthetic polypeptide
QSLEESGGRLVTPGTPLTLTCSVSGIDLSGYYMNWVRQAPGKGLEWIGVIGINGATYYASWAKGRFTISKTSSTTVDLKMTSLTTEDTATYFCARGDIWGPGTLVTVSS

SEQ ID NO: 76 - protein, 113 aa, Artificial Sequence; identical to SEQ ID NO: 68
  Description of Artificial Sequence Synthetic polypeptide

SEQ ID NO: 77 - protein, 111 aa, Artificial Sequence; identical to SEQ ID NO: 69
  Description of Artificial Sequence Synthetic polypeptide

SEQ ID NO: 78 - protein, 110 aa, Artificial Sequence
  Description of Artificial Sequence Synthetic polypeptide
QSVLTQPPSVSAAPGQKVTISCSGSSSNIGNNYVSWYQQLPGTAPKLLIYDNNKRPSGIPDRFSGSKSGTSTTLGITGLQTGDEADYYCGTWDSRLSAVVFGGGTKLTVL

SEQ ID NO: 79 - protein, 130 aa, Artificial Sequence [sequence omitted]

SEQ ID NO: 80 - protein, 113 aa, Artificial Sequence; identical to SEQ ID NO: 68
  Description of Artificial Sequence Synthetic polypeptide

SEQ ID NO: 81 - protein, 219 aa, Artificial Sequence
  Description of Artificial Sequence Synthetic polypeptide
QVLTQSPSSLSASVGDRVTINCQASQSVYHNTYLAWYQQKPGKVPKQLIYDASTLASGVPSRFSGSGSGTDFTLTISSLQPEDVATYYCLGSYDCTNGDCFVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 111 aa, Artificial Sequence; identical to SEQ ID NO: 69
  Description of Artificial Sequence Synthetic polypeptide

SEQ ID NO: 83 - protein, 441 aa, Artificial Sequence
  Description of Artificial Sequence Synthetic polypeptide
EVQLVESGGGLVQPGGSLRLSCAVSGIDLSGYYMNWVRQAPGKGLEWVGVIGINGATYYASWAKGRFTISRDNSKTTVYLQMNSLRAEDTAVYFCARGDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDARVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 13 aa, Artificial Sequence
  Description of Artificial Sequence Synthetic polypeptide
QASQSVYHNTYLA

SEQ ID NO: 85 - protein, 7 aa, Artificial Sequence
  Description of Artificial Sequence Synthetic polypeptide
DASTLAS

SEQ ID NO: 86 - protein, 13 aa, Artificial Sequence
  Description of Artificial Sequence Synthetic polypeptide
LGSYDCTNGDCFV

SEQ ID NO: 87 - protein, 5 aa, Artificial Sequence
  Description of Artificial Sequence Synthetic polypeptide
GYYMN

SEQ ID NO: 88 - protein, 15 aa, Artificial Sequence
  Description of Artificial Sequence Synthetic polypeptide
IGINGATYYASWAKG

SEQ ID NO: 89 - protein, 3 aa, Artificial Sequence
  Description of Artificial Sequence Synthetic polypeptide
GDI

SEQ ID NO: 90 - protein, 9 aa, Artificial Sequence
  Description of Artificial Sequence Synthetic polypeptide
QQGDALPPT

SEQ ID NO: 91 - protein, 10 aa, Artificial Sequence
  Description of Artificial Sequence Synthetic polypeptide
RASKDISKYL

SEQ ID NO: 92 - protein, 7 aa, Artificial Sequence
  Description of Artificial Sequence Synthetic polypeptide
YTSGYSH

SEQ ID NO: 93 - protein, 10 aa, Artificial Sequence
  Description of Artificial Sequence Synthetic polypeptide
GYTFGNYWMQ

SEQ ID NO: 94 - protein, 17 aa, Artificial Sequence
  Description of Artificial Sequence Synthetic polypeptide
AIYEGTGKTVYIQKFAD

SEQ ID NO: 95 - protein, 10 aa, Artificial Sequence
  Description of Artificial Sequence Synthetic polypeptide
LSDYVSGFGY

SEQ ID NO: 96 - protein, 107 aa, Artificial Sequences; identical to SEQ ID NO: 62
  Description of Artificial Sequence Synthetic polypeptide

SEQ ID NO: 97 - protein, 119 aa, Artificial Sequence; identical to SEQ ID NO: 63
  Description of Artificial Sequence Synthetic polypeptide

SEQ ID NO: 98 - protein, 214 aa, Artificial Sequence
  Description of Artificial Sequence Synthetic polypeptide
DIQMTQSPSSLSASVGDRVTITCRASKDISKYLNWYQQKPGKAPKLLIYYTSGYHSGVPSRFSGSGSGTDFTLTISSLQPEDFATYYCQQGDALPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 445 aa, Artificial Sequence
  Description of Artificial Sequence Synthetic polypeptide
QVQLVQSGAEVKKPGSSVKVSCKASGYTFGNYWMQWVRQAPGQGLEWMGAIYEGTGKTVYIQKFADRVTITADKSTSTAYMELSSLRSEDTAVYYCARLSDYVSGFGYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 100 - protein, 13 aa, Artificial Sequence
  Synthetic polypeptide
SGSSSNIGNNYVS

SEQ ID NO: 101 - protein, 7 aa, Artificial Sequence
  Synthetic polypeptide
DNNKRPS

SEQ ID NO: 102 - protein, 11 aa, Artificial Sequence
  Synthetic polypeptide
GTWDSRLSAVV

SEQ ID NO: 103 - protein, 5 aa, Artificial Sequence
  Synthetic polypeptide
SFGMH

SEQ ID NO: 104 - protein, 17 aa, Artificial Sequence
  Synthetic polypeptide
VISFDGSIKYSVDSVKG

SEQ ID NO: 105 - protein, 21 aa, Artificial Sequence
  Synthetic polypeptide
DRLNYYDSSGYYHYKYYGMAV

SEQ ID NO: 106 - protein, 110 aa, Artificial Sequence; identical to SEQ ID NO: 78
  Synthetic polypeptide

SEQ ID NO: 107 - protein, 130 aa, Artificial Sequence [sequence omitted]

SEQ ID NO: 108 - protein, 238 aa, Artificial Sequence
  Synthetic_polypeptide
MDMRVPAQLLGLLLLWLRGARCQSVLTQPPSVSAAPGQKVTISCSGSSSNIGNNYVSWYQQLPGTAPKLLIYDNNKRPSGIPDRFSGSKSGTSTTLGITGLQTGDEADYYCGTWDSRLSAVVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 109 - protein, 478 aa, Artificial Sequence
  Synthetic polypeptide
MDMRVPAQLLGLLLLWLRGARCQVQLVESGGGVVQPGRSLRLSCAASGFTFSSFGMHWVRQAPGKGLEWVAVISFDGSIKYSVDSVKGRFTISRDNSKNTLFLQMNSLRAEDTAVYYCARDRLNYYDSSGYYHYKYYGMAVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

(19) United States
(12) Patent Application Publication
(10) Pub. No.: US 2018/0111984 A1
(43) Pub. Date: Apr. 26, 2018

(54) TREATING CLUSTER HEADACHE

(71) Applicant: Teva Pharmaceuticals International GmbH, Jona (CH)

(72) Inventors: Marcelo Bigal, Doylestown, PA (US); Ernesto Aycardi, Andover, MA (US); Orit Cohenbarak, Haifa (IL)

(21) Appl. No.: 15/712,404

(22) Filed: Sep. 22, 2017

Related U.S. Application Data
(60) Provisional application No. 62/399,156, filed on Sep. 23, 2016.

(51) Int. Cl. C07K 16/18; A61P 25/00

(52) U.S. Cl. CPC C07K 16/18; A61K 2039/545; A61P 25/00

(57) ABSTRACT

Disclosed herein are methods of treating or reducing incidence of a cluster headache, e.g., chronic cluster headache and episodic cluster headache, and/or at least one secondary symptom associated with a cluster headache in a subject comprising administering to the subject a monoclonal antibody that modulates the CGRP pathway. Compositions for use in the disclosed methods are also provided. Antagonist antibody G1 and antibodies derived from G1 directed to CGRP are also described.

### CROSS REFERENCE TO RELATED APPLICATIONS

This application claims the benefit of priority of U.S. Application No. 62/399,156, filed on Sep. 23, 2016. The content of this prior application is hereby incorporated by reference in its entirety.

### BACKGROUND

Cluster headache (CH) is a primary headache disorder characterized by repetitive attacks of excruciating unilateral head pain and associated with cranial autonomic features (such as lacrimation, conjunctival injection, nasal congestion, nasal rhinorrhea, and partial Horner's syndrome) (Rozen, Curr Pain Headache Rep 2005; 9(2):135-40). CH attacks last up to 180 minutes and occur from once every other day to 8 times a day. Cluster periods usually last a few months (typically three months) followed by remission periods of months to years (Headache Classification Committee of the International Headache Society [IHS] 2013). A unique feature of CH is the circadian and circannual periodicity nature of the headache attacks. Peak time periods for daily CH onset are 0100 to 0200, 1300 to 1500, and after 2100, with night awakening attacks being more severe than those occurring during the day (Rozen, Curr Pain Headache Rep 2005; 9(2):135-40). Some patients tend to have seasonal attacks related to the duration of the photoperiod, with the highest incidence of attacks occurring in January or July with possible relation to solstices or equinoxes (Kudrow, Cephalalgia 1987; 7(Suppl6):76-8).

There are two forms of CH: episodic cluster headache (ECH), which is the most common form, where distinct pain-free periods lasting at least one month are evident, and chronic cluster headache (CCH) occurring for more than one year without remission or with remission periods lasting less than one month. About 10% to 15% of patients with CH have the CCH form (Headache Classification Committee of the IHS 2013).

The pathophysiology of CH is complex and not fully understood. Current theories implicate mechanisms such as vascular dilation, trigeminal nerve stimulation, and circadian effects. Histamine release, an increase in mast cells, genetic factors, and autonomic nervous system activation may also contribute (Weaver-Agostoni, J. Cluster headache, Am Fam Physician 2013; 188(2): 122-8). However, three major features of CH are the main focus for understanding its pathophysiological model: trigeminal distribution of the pain (including association with neuropeptide level changes), ipsilateral cranial autonomic features, and (circadian) episodic pattern of attacks (May, Lancet 2005; 366(9488): 843-55).

The excruciatingly severe unilateral pain is likely to be mediated by activation of the first (ophthalmic) division of the trigeminal nerve, whereas the autonomic symptoms such as lacrimation are due to activation of the cranial parasympathetic outflow from the seventh cranial nerve (Goadsby, Lancet Neurol 2002; 1(4): 251-7). When the trigeminal system becomes highly activated, the excitation spreads to the superior salivary nucleus, resulting in excitation from the sphenopalatine ganglion to parasympathetic nerves of intracranial large blood vessels, lacrimal glands, and nasal mucosa. As a result, ipsilateral autonomic symptoms such as Homer's sign, lacrimation, nasal congestion, and rhinorrhea are manifested (Goadsby, Lancet Neurol 2002; 1(4): 251-7; Japanese Headache Society, Clinical practice guideline for chronic headache 2013). Stimulation of the superior sagittal sinus activates the trigeminovascular pathway, and this also results in the release of neuropeptides such as calcitonin gene-related peptide (CGRP) and vasoactive intestinal peptide (VIP) in the external jugular vein. During attacks, the levels of CGRP and VIP are raised in cranial venous blood in all patients.

Worldwide, there are currently no approved medications for the preventive treatment of CH, and medications used off-label in clinical practice for this indication lack meaningful evidence to support their use. Among the medications used for ECH and CCH are short-course corticosteroids for transitional prophylaxis and verapamil (the most common first-line treatment), anti-seizure drugs (valproic acid, topiramate), ergotamine, melatonin, and capsaicin for maintenance prophylaxis. Lithium and deep-brain stimulation have also been used as preventive treatments for CCH (Weaver-Agostoni J. Cluster headache. Am Fam Physician 2013; 188(2): 122-8). Each of these treatment options is suboptimal due to limited evidence of efficacy, troublesome side effects, and/or an unfavorable risk to benefit ratio (Rozen, Curr Pain Headache Rep 2005; 9(2): 135-40, Weaver-Agostoni, J. Cluster headache. Am Fam Physician 2013; 188(2): 122-8).

Similar to migraine, the trigeminal system plays a pivotal role in the pathophysiology of CH. During CH attacks, activation of the trigeminal system causes neurovascular inflammation mediated by CGRP and other neuropeptides (Fanciullacci et al., Pain 1995; 60(2): 119-23; Fanciullacci et al. Brain 1997; 120(Pt 2): 283-8; Goadsby and Edvinsson, Brain 1994; 117(Pt 3): 427-34). In CH, the generator appears to be in the posterior grey matter of the hypothalamus (third neuron) (May et al. Nat Med 1999; 5(7): 836-8). Blocking CGRP in the peripheral ganglia of the trigeminal system may result in desensitization of the first and second neurons of the trigeminal system.

Monoclonal antibodies that modulate the CGRP pathway thus represent a class of promising therapeutic candidates for patients diagnosed with ECH or CCH.

### SUMMARY

Disclosed herein are anti-CGRP antagonist antibodies and methods of using the same for preventing, treating, or reducing incidence of a cluster headache, e.g., chronic cluster headache (CCH) and episodic cluster headache (ECH). Also disclosed herein are methods of preventing, treating, or reducing incidence of CCH and ECH in a subject comprising administering to the subject a monoclonal antibody that modulates the CGRP pathway.

Methods of preventing, treating, or reducing incidence of at least one secondary symptom associated with CCH and ECH in a subject comprising administering to the subject a monoclonal antibody that modulates the CGRP pathway are also provided. In some embodiments, the amount of the monoclonal antibody administered to the patient can be about 225 mg to about 1000 mg, e.g., about 675 mg or about 900 mg. Accordingly, in some aspects, the methods of preventing, treating, or reducing incidence of CCH and ECH in a subject can comprise administering to the subject a monoclonal antibody that modulates the CGRP pathway, wherein the amount of the monoclonal antibody administered to the patient can be about 225 mg to about 1000 mg, e.g., about 675 mg or about 900 mg. In other aspects, the methods of preventing, treating, or reducing incidence of at least one secondary symptom associated with CCH and ECH in a subject can comprise administering to the subject a monoclonal antibody that modulates the CGRP pathway are also provided, wherein the amount of the monoclonal antibody administered to the patient can be about 225 mg to about 1000 mg, e.g., about 675 mg or about 900 mg. In one embodiment, the dosing regimen comprises administering an initial antibody dose of about 675 mg subcutaneously, followed by a monthly antibody dose of about 225 mg subcutaneously for, e.g., about two months, three months, four months, five months, six months, seven months, eight months, nine months, ten months, 11 months, or 12 months, or even a period of greater than one year (e.g., 18 months, two years, or three years). In one embodiment, the dosing regimen comprises administering an initial antibody dose of about 900 mg intravenously, followed by a monthly antibody dose of about 225 mg subcutaneously for, e.g., about two months, three months, four months, five months, six months, seven months, eight months, nine months, ten months, 11 months, 12 months, or even a period of greater than one year (e.g., 18 months, two years, or three years). Yet another dosing regimen comprises administering an initial dose of about 900 mg intravenously in an infusion over about 60 minutes, followed by doses of about 900 mg administered intravenously in an infusion over about 60 minutes every quarter for, e.g., about one year, two years, three years, four years, or five years.

As CH is considered one of the most severe forms of pain a person can experience, treatments that provide quick and lasting relief (i.e., for the duration of the cluster period) are a priority. The biological nature of the disease mandates the need for any treatment to desensitize the third order neuron, suggesting that high levels of blockade at the first neuron are necessary. Therefore, high doses are planned for the first or starting dose (e.g., 900 mg intravenously or 675 mg subcutaneously) in order to provide a rapid response. Following the high initial dose, monthly doses at 225 mg sc can be added for maintenance of efficacy. Based on modelling, the inclusion of a high initial (or starting) dose will allow patients to reach steady state faster.

Suitable administration schedules include, but are not limited to, monthly, quarterly, or a single dose. In some embodiments, the monoclonal antibody can be administered monthly. For example, the monoclonal antibody can be administered monthly for one, two, three, four, five, six, seven, eight, nine, ten, 11, 12, or more months. In some aspects, the monoclonal antibody can be administered monthly for three or more months. When administered monthly, the dose of the monoclonal antibody administered to the patient can be about 100 mg to about 1000 mg, for example 225 mg to about 900 mg. The amounts administered in a first month can differ from the amounts administered in subsequent months. For example, in a first month, the dose of the monoclonal antibody administered to the patient can be about 675 mg or about 900 mg (e.g., an initial or starting dose), and the dose of the monoclonal antibody administered monthly thereafter can be about 225 mg.

The monoclonal antibody can be administered as a single dose. When administered as a single dose, the dose of the monoclonal antibody administered to the patient can be about 675 mg to about 1000 mg.

The treating or reducing can comprise reducing the number of headache hours of any severity, reducing the number of monthly headache days of any severity, reducing the use of any acute headache medications (e.g., cluster-specific acute headache medications such as triptans and ergot compounds), reducing a 6-item Headache Impact Test (HIT-6) disability score, improving 12-Item Short Form Health Survey (SF-12) score (Ware et al., Med Care 4:220-233, 1996), reducing Patient Global Impression of Change (PGIC) score (Hurst et al., J Manipulative Physiol Ther 27:26-35, 2004), improving Sport ConCuSSion ASSeSment tool 3 (SCAT-3) score (McCrory et al. British Journal of Sports Medicine 47:263-266, 2013), or any combination thereof. In some embodiments, the number of monthly headache days can be reduced for at least seven days after a single administration.

In some embodiments, monthly headache hours experienced by the subject after said administering is reduced by 40 or more hours (e.g., 45, 50, 55, 60, 65, 70, 75, 80, or more) from a pre-administration level in the subject. Monthly headache hours may be reduced by more than 60 hours. In some embodiments, monthly headache hours experienced by the subject after said administering are reduced by 25% or more (e.g., 30%, 35%, 40%, 45%, 50%, or more) relative to a pre-administration level in the subject. Monthly headache hours may be reduced by 40% or more. In some embodiments, monthly headache days experienced by the subject after said administering is reduced by three or more days (e.g., 3, 4, 5, 6, 7, 8, 9, 10, 11, 12, 13, 14, 15, 16, 17, 18, 19, 20, or more days) from a pre-administration level in the subject. In some embodiments, the number of monthly headache days can be reduced by at least about 50% from a pre-administration level in the subject. Thus, in some aspects, the number of monthly headache days can be reduced by at least about 50%, at least about 55%, at least about 60%, at least about 65%, at least about 70%, at least about 75%, at least about 80%, or at least about 90%.

In some embodiments, the administering can be subcutaneous administration. In some embodiments, the administering can be intravenous administration. In some embodiments, the administering can comprise utilizing a pre-filled syringe, pre-filled syringe with a needle safety device, injection pen, or auto-injector comprising a dose of the monoclonal antibody. In some embodiments, the monoclonal antibody can be formulated at a concentration of at least 150 mg/mL. In some embodiments, the monoclonal antibody can be administered in a volume of less than 2 mL, e.g., about 1.5 mL.

In some embodiments, the method further comprises administering to the subject a second agent simultaneously or sequentially with the monoclonal antibody. The second agent can be any of 5-HT1 agonists, triptans, ergot alkaloids, and non-steroidal anti-inflammatory drugs. In some embodiments, the second agent is an agent taken by the subject prophylactically. In some embodiments, monthly use of the second agent by the subject is decreased by at least about 15%, e.g., at least 16%, 17%, 18%, 20%, 22%, 25%, 28%, 30%, 35%, 40%, 45%, 50%, 55%, 60%, 65%, 70%, 75%, 80%, 85%, 90%, or at least about 95%, after administering the monoclonal antibody. In some embodiments, the second agent is a triptan.

In some embodiments, the subject is a human.

The monoclonal antibody can be an anti-CGRP antagonist antibody. In some embodiments, the monoclonal antibody is a human or humanized monoclonal antibody. In some embodiments, the monoclonal antibody comprises (a) an antibody having a CDR H1 as set forth in SEQ ID NO:3; a CDR H2 as set forth in SEQ ID NO:4; a CDR H3 as set forth in SEQ ID NO:5; a CDR L1 as set forth in SEQ ID NO:6; a CDR L2 as set forth in SEQ ID NO:7; and a CDR L3 as set forth in SEQ ID NO:8; or (b) a variant of an antibody according to (a) as shown in Table 6.

Also disclosed are methods of decreasing a number of monthly headache hours experienced by a subject having CCH and ECH. In one embodiment, the method comprises administering to the subject an amount of a monoclonal antibody that modulates the CGRP pathway, wherein the monoclonal antibody is in an amount effective to decrease the number of monthly headache hours by at least 20 (e.g., 25, 30, 35, 40, 45, 50, 55, 60, 65, 70 or more headache hours) after a single dose. In some embodiments, the number of monthly headache hours is reduced by at least about 50 hours. In one embodiment, the method comprises administering to the subject an amount of a monoclonal antibody that modulates the CGRP pathway, wherein the monoclonal antibody is in an amount effective to decrease the number of monthly headache hours by at least 15% (e.g., 20%, 25%, 30%, 35%, 40%, or more) after a single dose. In some embodiments, the number of monthly headache hours is reduced by at least about 30%. In some embodiments, the monoclonal antibody is an anti-CGRP antagonist antibody. In some embodiments, the amount of the monoclonal antibody administered to the patient is about 225 mg to about 1000 mg. In some embodiments, the monoclonal antibody is administered monthly. In some embodiments, the monoclonal antibody is administered as a single dose. In some embodiments, the administering is subcutaneous or intravenous administration. In some embodiments, the monoclonal antibody is formulated at a concentration of at least 150 mg/mL. In some embodiments, the monoclonal antibody is administered in a volume of less than 2 mL, e.g., about 1.5 mL. In some embodiments, the subject is human. In some embodiments, the monoclonal antibody is human or humanized. In some embodiments, the monoclonal antibody comprises (a) an antibody having a CDR H1 as set forth in SEQ ID NO:3; a CDR H2 as set forth in SEQ ID NO:4; a CDR H3 as set forth in SEQ ID NO:5; a CDR L1 as set forth in SEQ ID NO:6; a CDR L2 as set forth in SEQ ID NO:7; and a CDR L3 as set forth in SEQ ID NO:8; or (b) a variant of an antibody according to (a) as shown in Table 6.

Also disclosed are methods of decreasing a number of monthly headache days experienced by a subject having CCH and ECH. In one embodiment, the method comprises administering to the subject an amount of a monoclonal antibody that modulates the CGRP pathway, wherein the monoclonal antibody is in an amount effective to decrease the number of monthly headache days by at least 3 (e.g., 3, 4, 5, 6, 7, 8, 9, 10, 11, 12, 13, 14, 15, 16, 17, 18, 19, 20 or more headache days) after a single dose. In some embodiments, the number of monthly headache days is reduced by at least about 6 headache days. In some embodiments, the number of monthly headache days can be reduced by at least about 50% from a pre-administration level in the subject. Thus, in some aspects, the number of monthly headache days can be reduced by at least about 50%, at least about 55%, at least about 60%, at least about 65%, at least about 70%, at least about 75%, at least about 80%, or at least about 90%. In some embodiments, the monoclonal antibody is an anti-CGRP antagonist antibody. In some embodiments, the amount of the monoclonal antibody administered to the patient is about 225 mg to about 1000 mg. In some embodiments, the monoclonal antibody is administered monthly. In some embodiments, the monoclonal antibody is administered as a single dose. In some embodiments, the administering is subcutaneous or intravenous administration. In some embodiments, the monoclonal antibody is formulated at a concentration of at least 150 mg/mL. In some embodiments, wherein the monoclonal antibody is administered in a volume of less than 2 mL, e.g., about 1.5 mL. In some embodiments, the subject is human. In some embodiments, the monoclonal antibody is human or humanized. In some embodiments, the monoclonal antibody comprises (a) an antibody having a CDR H1 as set forth in SEQ ID NO:3; a CDR H2 as set forth in SEQ ID NO:4; a CDR H3 as set forth in SEQ ID NO:5; a CDR L1 as set forth in SEQ ID NO:6; a CDR L2 as set forth in SEQ ID NO:7; and a CDR L3 as set forth in SEQ ID NO:8; or (b) a variant of an antibody according to (a) as shown in Table 6.

Also disclosed are methods of decreasing use of any acute headache medication in a subject having CCH or ECH, comprising administering to the subject a monoclonal antibody (e.g., anti-CGRP antagonist antibody) that modulates the CGRP pathway, wherein the monoclonal antibody is in an amount effective to decrease monthly use of the anti-headache medication by the subject by at least 15% (e.g., 20%, 25%, 30%, 35%, 40%, or more). In some embodiments, the anti-headache medication is selected from the group consisting of 5-HT1 agonists, triptans, opiates, β-adrenergic antagonists, ergot alkaloids, and non-steroidal anti-inflammatory drugs (NSAIDs). In some embodiments, the anti-headache medication is a triptan. In some embodiments, the amount of the monoclonal antibody administered to the patient is about 225 mg to about 1000 mg, e.g., about 675 mg or about 900 mg. In some embodiments, the monoclonal antibody is administered monthly. In some embodiments, the monoclonal antibody is administered as a single dose. In some embodiments, the administering is subcutaneous or intravenous administration. In some embodiments, the monoclonal antibody is formulated at a concentration of at least 150 mg/mL (e.g., 225 mg/1.5 mL). In some embodiments, wherein the monoclonal antibody is administered in a volume of less than 2 mL, e.g., about 1.5 mL. In some embodiments, the subject is human. In some embodiments, the monoclonal antibody is human or humanized. In some embodiments, the monoclonal antibody comprises (a) an antibody having a CDR H1 as set forth in SEQ ID NO:3; a CDR H2 as set forth in SEQ ID NO:4; a CDR H3 as set forth in SEQ ID NO:5; a CDR L1 as set forth in SEQ ID NO:6; a CDR L2 as set forth in SEQ ID NO:7; and a CDR L3 as set forth in SEQ ID NO:8; or (b) a variant of an antibody according to (a) as shown in Table 6.

In one aspect, the invention provides a method of preventing, treating, or reducing incidence of CCH or ECH in a subject comprising administering to the subject a single dose of a monoclonal antibody (e.g., monoclonal anti-CGRP-antagonist antibody) in an amount that modulates the CGRP pathway, wherein the amount of the monoclonal antibody is about 225 mg to about 1000 mg, e.g., about 675 mg or about 900 mg.

In a further embodiment, the invention provides methods for preventing, treating, ameliorating, controlling, reducing incidence of, or delaying the development or progression of CCH or ECH in an individual comprising administering to the individual an effective amount of an anti-CGRP antagonist antibody in combination with at least one additional agent useful for treating the CCH or ECH. Such additional agents include 5-HT1-like agonists (and agonists acting at other 5-HT1 sites), and non-steroidal anti-inflammatory drugs (NSAIDs).

Examples of 5-HT1 agonists that can be used in combination with an anti-CGRP antibody include a class of compounds known as triptans, such as sumatriptan, zolmitriptan, naratriptan, rizatriptan, eletriptan, almotriptan, and frovatriptan. Ergot alkaloids and related compounds are also known to have 5-HT agonist activity and have been used to treat headaches. Included among these compounds are ergotamine tartrate, ergonovine maleate, and ergoloid mesylates (e.g., dihydroergocornine, dihydroergocristine, dihydroergocryptine, and dihydroergotamine mesylate (DHE 45)).

Examples of NSAIDs that can be used in combination with an anti-CGRP antibody include aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, nabumetone, naproxen, oxaprozin, phenylbutazone, piroxicam, sulindac, tolmetin or zomepirac, cyclooxygenase-2 (COX-2) inhibitors, celecoxib; rofecoxib; meloxicam; JTE-522; L-745,337; NS398; or a pharmaceutically acceptable salt thereof.

In one embodiment, the anti-CGRP antagonist antibody used in any of the methods described above is any of the antibodies as described herein.

In some embodiments, the anti-CGRP antagonist antibody recognizes a human CGRP. In some embodiments, the anti-CGRP antagonist antibody binds to both human α-CGRP and β-CGRP. In some embodiments, the anti-CGRP antagonist antibody binds human and rat CGRP. In some embodiments, the anti-CGRP antagonist antibody binds the C-terminal fragment having amino acids 25-37 of CGRP. In some embodiments, the anti-CGRP antagonist antibody binds a C-terminal epitope within amino acids 25-37 of CGRP.

In some embodiments, the anti-CGRP antagonist antibody is a monoclonal antibody. In some embodiments, the anti-CGRP antagonist antibody is humanized. In some embodiments, the antibody is human. In some embodiments, the anti-CGRP antagonist antibody is antibody G1 (as described herein). In some embodiments, the anti-CGRP antagonist antibody comprises one or more CDR(s) (such as one, two, three, four, five, or, in some embodiments, all six CDRs) of antibody G1 or variants of G1 shown in Table 6. In still other embodiments, the anti-CGRP antagonist antibody comprises the amino acid sequence of the heavy chain variable region shown in FIG. 5 (SEQ ID NO:1) and the amino acid sequence of the light chain variable region shown in FIG. 5 (SEQ ID NO:2).

In some embodiments, the antibody comprises a modified constant region, such as a constant region that is immunologically inert (including partially immunologically inert), e.g., does not trigger complement mediated lysis, does not stimulate antibody-dependent cell mediated cytotoxicity (ADCC), does not activate microglia, or having reduced one or more of these activities. In some embodiments, the constant region is modified as described in Eur. J. Immunol. (1999) 29:2613-2624; PCT Application No. PCT/GB99/01441; and/or UK Patent Application No. 9809951.8. In other embodiments, the antibody comprises a human heavy chain IgG2 constant region comprising the following mutations: A330P331 to S330S331 (amino acid numbering with reference to the wildtype IgG2 sequence). Eur. J. Immunol. (1999) 29:2613-2624. In some embodiments, the heavy chain constant region of the antibody is a human heavy chain IgG1 with any of the following mutations: 1) A327A330P331 to G327S330S331; 2) E233L234L235G236 (SEQ ID NO:48) to P233V234A235 with G236 deleted; 3) E233L234L235 to P233V234A235; 4) E233L234L235G236A327A330P331 (SEQ ID NO:49) to P233V234A235G327S330S331 (SEQ ID NO:50) with G236 deleted; 5) E233L234L235A327A330P331 (SEQ ID NO:51) to P233V234A235G327S330S331 (SEQ ID NO:50); and 6) N297 to A297 or any other amino acid except N. In some embodiments, the heavy chain constant region of the antibody is a human heavy chain IgG4 with any of the following mutations: E233F234L235G236 (SEQ ID NO:52) to P233V234A235 with G236 deleted; E233F234L235 to P233V234A235; and 5228L235 to P228E235.

In still other embodiments, the constant region is aglycosylated for N-linked glycosylation. In some embodiments, the constant region is aglycosylated for N-linked glycosylation by mutating the oligosaccharide attachment residue (such as Asn297) and/or flanking residues that are part of the N-glycosylation recognition sequence in the constant region. In some embodiments, the constant region is aglycosylated for N-linked glycosylation. The constant region may be aglycosylated for N-linked glycosylation enzymatically or by expression in a glycosylation deficient host cell.

The binding affinity (K_{D}) of an anti-CGRP antagonist antibody to CGRP (such as human α-CGRP as measured by surface plasmon resonance at an appropriate temperature, such as 25 or 37° C.) can be about 0.02 to about 200 nM. In some embodiments, the binding affinity is any of about 200 nM, about 100 nM, about 50 nM, about 10 nM, about 1 nM, about 500 pM, about 100 pM, about 60 pM, about 50 pM, about 20 pM, about 15 pM, about 10 pM, about 5 pM, or about 2 pM. In some embodiments, the binding affinity is less than any of about 250 nM, about 200 nM, about 100 nM, about 50 nM, about 10 nM, about 1 nM, about 500 pM, about 100 pM, or about 50 pM. In some embodiments, the binding affinity is less than about 50 nM.

The anti-CGRP antagonist antibody may be administered prior to, during, and/or after a cluster headache. In some embodiments, the anti-CGRP antagonist antibody is administered prior to the CCH or ECH attack. Administration of an anti-CGRP antagonist antibody can be by any means known in the art, including: orally, intravenously, subcutaneously, intraarterially, intramuscularly, intranasally (e.g., with or without inhalation), intracardially, intraspinally, intrathoracically, intraperitoneally, intraventricularly, sublingually, transdermally, and/or via inhalation. Administration may be systemic, e.g., intravenously, or localized. In some embodiments, an initial dose and one or more additional doses are administered the same way, i.e., subcutaneously or intravenously. In some embodiments, the one or more additional doses are administered in a different way than the initial dose, i.e., the initial dose may be administered intravenously and the one or more additional doses may be administered subcutaneously.

In some embodiments, the anti-CGRP antagonist antibody may be administered in conjunction with another agent, such as another agent for treating CCH or ECH.

In another aspect, the invention provides use of an anti-CGRP antagonist antibody for the manufacture of a medicament for use in any of the methods described herein, for example, for preventing, treating, or reducing CCH or ECH.

In another aspect, the invention provides a pharmaceutical composition for preventing, treating, or reducing CCH or ECH comprising an effective amount of an anti-CGRP antagonist antibody, in combination with one or more pharmaceutically acceptable excipients.

In another aspect, the invention provides a kit for use in any of the methods described herein. In some embodiments, the kit comprises a container, a composition comprising an anti-CGRP antagonist antibody described herein, in combination with a pharmaceutically acceptable carrier, and instructions for using the composition in any of the methods described herein.

The present invention also provides anti-CGRP antagonist antibodies and polypeptides derived from antibody G1 or its variants shown in Table 6. Accordingly, in one aspect, the invention provides an antibody G1 (interchangeably termed “G1” and “TEV-48125”) that is produced by expression vectors having ATCC Accession Nos. PTA-6866 and PTA-6867. For example, in one embodiment is an antibody comprising a heavy chain produced by the expression vector with ATCC Accession No. PTA-6867. In a further embodiment is an antibody comprising a light chain produced by the expression vector with ATCC Accession No. PTA-6866. The amino acid sequences of the heavy chain and light chain variable regions of G1 are shown in FIG. 5. The complementarity determining region (CDR) portions of antibody G1 (including Chothia and Kabat CDRs) are also shown in FIG. 5. It is understood that reference to any part of or entire region of G1 encompasses sequences produced by the expression vectors having ATCC Accession Nos. PTA-6866 and PTA-6867, and/or the sequences depicted in FIG. 5. In some embodiments, the invention also provides antibody variants of G1 with amino acid sequences depicted in Table 6.

In some embodiments, the antibody comprises a V_{H} domain that is at least 85%, at least 86%, at least 87%, at least 88%, at least 89%, at least 90%, at least 91%, at least 92%, at least 93%, at least 94%, at least 95%, at least 96%, at least 97% at least 98%, at least 99% or 100% identical in amino acid sequence to SEQ ID NO:1.

In some embodiments, the antibody comprises a V_{L} domain that is at least 85%, at least 86%, at least 87%, at least 88%, at least 89%, at least 90%, at least 91%, at least 92%, at least 93%, at least 94%, at least 95%, at least 96%, at least 97% at least 98%, at least 99% or 100% identical in amino acid sequence to SEQ ID NO:2.

In some embodiments, the antibody comprises a heavy chain sequence that is at least 85%, at least 86%, at least 87%, at least 88%, at least 89%, at least 90%, at least 91%, at least 92%, at least 93%, at least 94%, at least 95%, at least 96%, at least 97% at least 98%, at least 99% or 100% identical in amino acid sequence to SEQ ID NO:11.

In some embodiments, the antibody comprises a light chain sequence that is at least 85%, at least 86%, at least 87%, at least 88%, at least 89%, at least 90%, at least 91%, at least 92%, at least 93%, at least 94%, at least 95%, at least 96%, at least 97% at least 98%, at least 99% or 100% identical in amino acid sequence to SEQ ID NO:12.

In some embodiments, the antibody comprises a fragment or a region of the antibody G1 or its variants shown in Table 6. In one embodiment, the fragment is a light chain of the antibody G1. In another embodiment, the fragment is a heavy chain of the antibody G1. In yet another embodiment, the fragment contains one or more variable regions from a light chain and/or a heavy chain of the antibody G1. In yet another embodiment, the fragment contains one or more variable regions from a light chain and/or a heavy chain shown in FIG. 5. In yet another embodiment, the fragment contains one or more CDRs from a light chain and/or a heavy chain of the antibody G1.

In some embodiments, the polypeptide (such as an antibody) comprises the amino acid sequence of KASKXaaVXaaTYVS (SEQ ID NO:53), wherein Xaa at position 5 is R, W, G, L, or N; and wherein Xaa at position 7 is T, A, D, G, R, S, W, or V. In some embodiments, the amino acid sequence of KASKXaaVXaaTYVS (SEQ ID NO:53) is CDR1 of an antibody light chain.

In some embodiments, the polypeptide (such as an antibody) comprises the amino acid sequence of XaaXaaSNRYXaa (SEQ ID NO:54), wherein Xaa at position 1 is G or A; wherein Xaa at position 2 is A or H; and wherein Xaa at position 7 is L, T, I, or S. In some embodiments, the amino acid sequence of XaaXaaSNRYXaa (SEQ ID NO:54) is CDR2 of an antibody light chain.

In some embodiments, the polypeptide (such as an antibody) comprises the amino acid sequence of EIRSXaaSDXaaXaaATXaaYAXaaAVKG (SEQ ID NO:55), wherein Xaa at position 5 is E, R, K, Q, or N; wherein Xaa at position 8 is A, G, N, E, H, S, L, R, C, F, Y, V, D, or P; wherein Xaa at position 9 is S, G, T, Y, C, E, L, A, P, I, N, R, V, D, or M; wherein Xaa at position 12 is H or F; wherein Xaa at position 15 is E or D. In some embodiments, the amino acid sequence of EIRSXaaSDXaaXaaATXaaYAXaaAVKG (SEQ ID NO:55) is CDR2 of an antibody heavy chain.

In some embodiments, the antibody is a human antibody. In other embodiments, the antibody a humanized antibody. In some embodiments, the antibody is monoclonal. In some embodiments, the antibody (or polypeptide) is isolated. In some embodiments, the antibody (or polypeptide) is substantially pure.

The heavy chain constant region of the antibodies may be from any types of constant region, such as IgG, IgM, IgD, IgA, and IgE; and any isotypes, such as IgG1, IgG2, IgG3, and IgG4.

In some embodiments, the antibody comprises a modified constant region as described herein.

In yet another aspect, the invention features uses of a monoclonal antibody comprising a CDR H1 as set forth in SEQ ID NO:3; a CDR H2 as set forth in SEQ ID NO:4; a CDR H3 as set forth in SEQ ID NO:5; a CDR L1 as set forth in SEQ ID NO:6; a CDR L2 as set forth in SEQ ID NO:7; and a CDR L3 as set forth in SEQ ID NO:8, for the manufacture of a medicament for treatment of a chronic cluster headache.

In another aspect, the invention provides uses of a monoclonal antibody comprising a CDR H1 as set forth in SEQ ID NO:3; a CDR H2 as set forth in SEQ ID NO:4; a CDR H3 as set forth in SEQ ID NO:5; a CDR L1 as set forth in SEQ ID NO:6; a CDR L2 as set forth in SEQ ID NO:7; and a CDR L3 as set forth in SEQ ID NO:8, for the manufacture of a medicament for treatment of an episodic cluster headache.

In one aspect, the invention provides a composition for use of the monoclonal antibody in accordance with any of the methods described herein.

In one aspect, the invention provides a composition for use of the monoclonal antibody in decreasing a number of monthly headache hours experienced by a subject. In one embodiment, the use comprises administering to the subject an amount of a monoclonal antibody that modulates the CGRP pathway, wherein the monoclonal antibody is in an amount effective to decrease the number of monthly headache hours by at least 20 (e.g., 25, 30, 35, 40, 45, 50, 55, 60, 65, 70 or more headache hours) after a single dose. In some embodiments, the number of monthly headache hours is reduced by at least about 50 hours. In one embodiment, the use comprises administering to the subject an amount of a monoclonal antibody that modulates the CGRP pathway, wherein the monoclonal antibody is in an amount effective to decrease the number of monthly headache hours by at least 15% (e.g., 20%, 25%, 30%, 35%, 40%, or more) after a single dose. In some embodiments, the number of monthly headache hours is reduced by at least about 30%. In some embodiments, the monoclonal antibody is an anti-CGRP antagonist antibody. In some embodiments, the amount of the monoclonal antibody administered to the patient is about 675 mg to about 1000 mg. In some embodiments, the monoclonal antibody is administered monthly. In some embodiments, the monoclonal antibody is administered as a single dose. In some embodiments, the administering is subcutaneous or intravenous administration. In some embodiments, the monoclonal antibody is formulated at a concentration of at least 150 mg/mL. In some embodiments, wherein the monoclonal antibody is administered in a volume of less than 2 mL. In some embodiments, the subject is human. In some embodiments, the monoclonal antibody is human or humanized. In some embodiments, the monoclonal antibody comprises (a) an antibody having a CDR H1 as set forth in SEQ ID NO:3; a CDR H2 as set forth in SEQ ID NO:4; a CDR H3 as set forth in SEQ ID NO:5; a CDR L1 as set forth in SEQ ID NO:6; a CDR L2 as set forth in SEQ ID NO:7; and a CDR L3 as set forth in SEQ ID NO:8; or (b) a variant of an antibody according to (a) as shown in Table 6.

In one aspect, the invention provides a composition for use of the monoclonal antibody in decreasing a number of monthly headache days experienced by a subject. In one embodiment, the use comprises administering to the subject an amount of a monoclonal antibody that modulates the CGRP pathway, wherein the monoclonal antibody is in an amount effective to decrease the number of monthly headache days by at least 3 (e.g., 3, 4, 5, 6, 7, 8, 9, 10, 11, 12, 13, 14, 15, 16, 17, 18, 19, 20 or more headache days) after a single dose. In some embodiments, the number of monthly headache days is reduced by at least about 6 headache days. In some embodiments, the monoclonal antibody is an anti-CGRP antagonist antibody. In some embodiments, the amount of the monoclonal antibody administered to the patient is about 675 mg to about 1000 mg. In some embodiments, the monoclonal antibody is administered monthly. In some embodiments, the monoclonal antibody is administered as a single dose. In some embodiments, the administering is subcutaneous or intravenous administration. In some embodiments, the monoclonal antibody is formulated at a concentration of at least 150 mg/mL. In some embodiments, wherein the monoclonal antibody is administered in a volume of less than 2 mL, e.g., about 1.5 mL. In some embodiments, the subject is human. In some embodiments, the monoclonal antibody is human or humanized. In some embodiments, the monoclonal antibody comprises (a) an antibody having a CDR H1 as set forth in SEQ ID NO:3; a CDR H2 as set forth in SEQ ID NO:4; a CDR H3 as set forth in SEQ ID NO:5; a CDR L1 as set forth in SEQ ID NO:6; a CDR L2 as set forth in SEQ ID NO:7; and a CDR L3 as set forth in SEQ ID NO:8; or (b) a variant of an antibody according to (a) as shown in Table 6.

In one aspect, the invention provides a composition for use of the monoclonal antibody in decreasing use of any acute headache medication in a subject, comprising administering to the subject a monoclonal antibody (e.g., anti-CGRP antagonist antibody) that modulates the CGRP pathway, wherein the monoclonal antibody is in an amount effective to decrease monthly use of the acute headache medication by the subject by at least 15% (e.g., 20%, 25%, 30%, 35%, 40%, or more). In some embodiments, the anti-headache medication is selected from the group consisting of 5-HT1 agonists, triptans, opiates, β-adrenergic antagonists, ergot alkaloids, and non-steroidal anti-inflammatory drugs (NSAIDs). In some embodiments, the anti-headache medication is a triptan. In some embodiments, the amount of the monoclonal antibody administered to the patient is about 675 mg to about 1000 mg. In some embodiments, the monoclonal antibody is administered monthly. In some embodiments, the monoclonal antibody is administered as a single dose. In some embodiments, the administering is subcutaneous or intravenous administration. In some embodiments, the monoclonal antibody is formulated at a concentration of at least 150 mg/mL. In some embodiments, wherein the monoclonal antibody is administered in a volume of less than 2 mL, e.g., about 1.5 mL. In some embodiments, the subject is human. In some embodiments, the monoclonal antibody is human or humanized. In some embodiments, the monoclonal antibody comprises (a) an antibody having a CDR H1 as set forth in SEQ ID NO:3; a CDR H2 as set forth in SEQ ID NO:4; a CDR H3 as set forth in SEQ ID NO:5; a CDR L1 as set forth in SEQ ID NO:6; a CDR L2 as set forth in SEQ ID NO:7; and a CDR L3 as set forth in SEQ ID NO:8; or (b) a variant of an antibody according to (a) as shown in Table 6.

In one aspect, the invention provides a composition for use of the monoclonal antibody in of preventing, treating, or reducing incidence of CCH or ECH in a subject comprising administering to the subject a single dose of a monoclonal antibody (e.g., monoclonal anti-CGRP-antagonist antibody) in an amount that modulates the CGRP pathway, wherein the amount of the monoclonal antibody administered to the patient is about 675 mg to about 1000 mg.

### BRIEF DESCRIPTION OF THE DRAWINGS

FIG. 1 is a table showing binding affinities of 12 murine antibodies for different alanine substituted human α-CGRP fragments. Binding affinities were measured at 25° C. using Biacore by flowing Fabs across CGRPs on the chip. The boxed values represent the loss in affinity of alanine mutants relative to parental fragment, 25-37 (italic), except K35A, which was derived from a 19-37 parent. “^{a}” indicates affinities for 19-37 and 25-37 fragments are the mean average±standard deviation of two independent measurements on different sensor chips. “^{b}” indicates these interactions deviated from a simple bimolecular interaction model due to a biphasic offrate, so their affinities were determined using a conformational change model. Grey-scale key: white (1.0) indicates parental affinity; light grey (less than 0.5) indicates higher affinity than parent; dark grey (more than 2) indicates lower affinity than parent; and black indicates that no binding was detected.

FIGS. 2A and 2B show the effect of administering CGRP 8-37 (400 nmol/kg), antibody 4901 (25 mg/kg), and antibody 7D11 (25 mg/kg) on skin blood flow measured as blood cell flux after electrical pulse stimulation for 30 seconds. CGRP 8-37 was administered intravenously (iv) 3-5 min before electrical pulse stimulation. Antibodies were administered intraperitoneal (IP) 72 hours before electrical pulse stimulation. Each point in the graphs represents AUC of one rat treated under the conditions as indicated. Each line in the graphs represents average AUC of rats treated under the condition as indicated. AUC (area under the curve) equals to Δflux×Δtime. “Δflux” represents the change of flux units after the electrical pulse stimulation; and “Δtime” represents the time period taken for the blood cell flux level to return to the level before the electrical pulse stimulation.

FIG. 3 shows the effect of administering different dosage of antibody 4901 (25 mg/kg, 5 mg/kg, 2.5 mg/kg, or 1 mg/kg) on skin blood flow measured as blood cell flux after electrical pulse stimulation for 30 seconds. Antibodies were administered intravenously (IV) 24 hours before electrical pulse stimulation. Each point in the graph represents AUC of one rat treated under the conditions as indicated. The line in the graph represents average AUC of rats treated under the condition as indicated.

FIGS. 4A and 4B show the effect of administering antibody 4901 (1 mg/kg or 10 mg/kg, i.v.), antibody 7E9 (10 mg/kg, i.v.), and antibody 8B6 (10 mg/kg, i.v.) on skin blood flow measured as blood cell flux after electrical pulse stimulation for 30 seconds. Antibodies were administered intravenously (i.v.) followed by electrical pulse stimulation at 30 min, 60 min, 90 min, and 120 min after antibody administration. Y axis represents percent of AUC as compared to level of AUC when no antibody was administered (time 0). X axis represents time (minutes) period between the administration of antibodies and electrical pulse stimulation. “*” indicates P<0.05, and “**” indicates P<0.01, as compared to time 0. Data were analyzed using one-way ANOVA with a Dunnett's Multiple comparison test.

FIG. 5 shows the amino acid sequence of the heavy chain variable region (SEQ ID NO:1) and light chain variable region (SEQ ID NO:2) of antibody G1. The Kabat CDRs are in bold text, and the Chothia CDRs are underlined. The amino acid residues for the heavy chain and light chain variable region are numbered sequentially.

FIG. 6 shows epitope mapping of antibody G1 by peptide competition using Biacore. N-biotinylated human α-CGRP was captured on SA sensor chip. G1 Fab (50 nM) in the absence of a competing peptide or pre-incubated for 1 hour with 10 μM of a competing peptide was flowed onto the chip. Binding of G1 Fab to the human α-CGRP on the chip was measured. Y axis represents percentage of binding blocked by the presence of the competing peptide compared with the binding in the absence of the competing peptide.

### DETAILED DESCRIPTION

In some aspects, the invention disclosed herein provides methods for preventing, treating, and/or reducing CCH or ECH in an individual by administering to the individual a therapeutically effective amount of an anti-CGRP antagonist antibody.

In some aspects, the invention disclosed herein also provides anti-CGRP antagonist antibodies and polypeptides derived from G1 or its variants shown in Table 6.

### General Techniques

The practice of the various aspects of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

### Definitions

As used herein, “about” when used in reference to numerical ranges, cutoffs, or specific values is used to indicate that the recited values may vary by up to as much as 10% from the listed value. Thus, the term “about” is used to encompass variations of ±10% or less, variations of ±5% or less, variations of ±1% or less, variations of ±0.5% or less, or variations of ±0.1% or less from the specified value.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)₂, Fv), single chain (ScFv), mutants thereof, fusion proteins comprising an antibody portion (such as domain antibodies), and any other modified configuration of the immunoglobulin molecule that comprises an antigen recognition site. An antibody includes an antibody of any class, such as IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

As used herein, “monoclonal antibody” refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, 1975, Nature, 256:495, or may be made by recombinant DNA methods such as described in U.S. Pat. No. 4,816,567. The monoclonal antibodies may also be isolated from phage libraries generated using the techniques described in McCafferty et al., 1990, Nature, 348:552-554, for example.

As used herein, “humanized” antibodies refer to forms of non-human (e.g., murine) antibodies that are specific chimeric immunoglobulins, immunoglobulin chains, or fragments thereof (such as Fv, Fab, Fab′, F(ab′)₂ or other antigen-binding subsequences of antibodies) that contain minimal sequence derived from non-human immunoglobulin. For the most part, humanized antibodies are human immunoglobulins (recipient antibody) in which residues from a complementarity determining region (CDR) of the recipient are replaced by residues from a CDR of a non-human species (donor antibody) such as mouse, rat, or rabbit having the desired specificity, affinity, and, biological activity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, six) which are altered with respect to the original antibody, which are also termed one or more CDRs “derived from” one or more CDRs from the original antibody.

As used herein, “human antibody” means an antibody having an amino acid sequence corresponding to that of an antibody produced by a human and/or has been made using any of the techniques for making human antibodies known in the art or disclosed herein. This definition of a human antibody includes antibodies comprising at least one human heavy chain polypeptide or at least one human light chain polypeptide. One such example is an antibody comprising murine light chain and human heavy chain polypeptides. Human antibodies can be produced using various techniques known in the art. In one embodiment, the human antibody is selected from a phage library, where that phage library expresses human antibodies (Vaughan et al., 1996, Nature Biotechnology, 14:309-314; Sheets et al., 1998, PNAS, (USA) 95:6157-6162; Hoogenboom and Winter, 1991, J. Mol. Biol., 227:381; Marks et al., 1991, J. Mol. Biol., 222:581). Human antibodies can also be made by introducing human immunoglobulin loci into transgenic animals, e.g., mice in which the endogenous immunoglobulin genes have been partially or completely inactivated. This approach is described in U.S. Pat. Nos. 5,545,807; 5,545,806; 5,569,825; 5,625,126; 5,633,425; and 5,661,016. Alternatively, the human antibody may be prepared by immortalizing human B lymphocytes that produce an antibody directed against a target antigen (such B lymphocytes may be recovered from an individual or may have been immunized in vitro). See, e.g., Cole et al., Monoclonal Antibodies and Cancer Therapy, Alan R. Liss, p. 77 (1985); Boerner et al., 1991, J. Immunol., 147 (1):86-95; and U.S. Pat. No. 5,750,373.

As used herein, the term “calcitonin gene-related peptide” and “CGRP” refers to any form of calcitonin gene-related peptide and variants thereof that retain at least part of the activity of CGRP. For example, CGRP may be α-CGRP or β-CGRP. As used herein, CGRP includes all mammalian species of native sequence CGRP, e.g., human, canine, feline, equine, and bovine.

As used herein, an “anti-CGRP antagonist antibody” (interchangeably termed “anti-CGRP antibody”) refers to an antibody that is able to bind to CGRP and inhibit CGRP biological activity and/or downstream pathway(s) mediated by CGRP signaling. An anti-CGRP antagonist antibody encompasses antibodies that modulate, block, antagonize, suppress or reduce (including significantly) CGRP biological activity, or otherwise antagonize the CGRP pathway, including downstream pathways mediated by CGRP signaling, such as receptor binding and/or elicitation of a cellular response to CGRP. For purpose of the present invention, it will be explicitly understood that the term “anti-CGRP antagonist antibody” encompasses all the previously identified terms, titles, and functional states and characteristics whereby CGRP itself, CGRP biological activity (including but not limited to its ability to mediate any aspect of headache), or the consequences of the biological activity, are substantially nullified, decreased, or neutralized in any meaningful degree. In some embodiments, an anti-CGRP antagonist antibody binds CGRP and prevents CGRP binding to a CGRP receptor. In other embodiments, an anti-CGRP antibody binds CGRP and prevents activation of a CGRP receptor. Examples of anti-CGRP antagonist antibodies are provided herein.

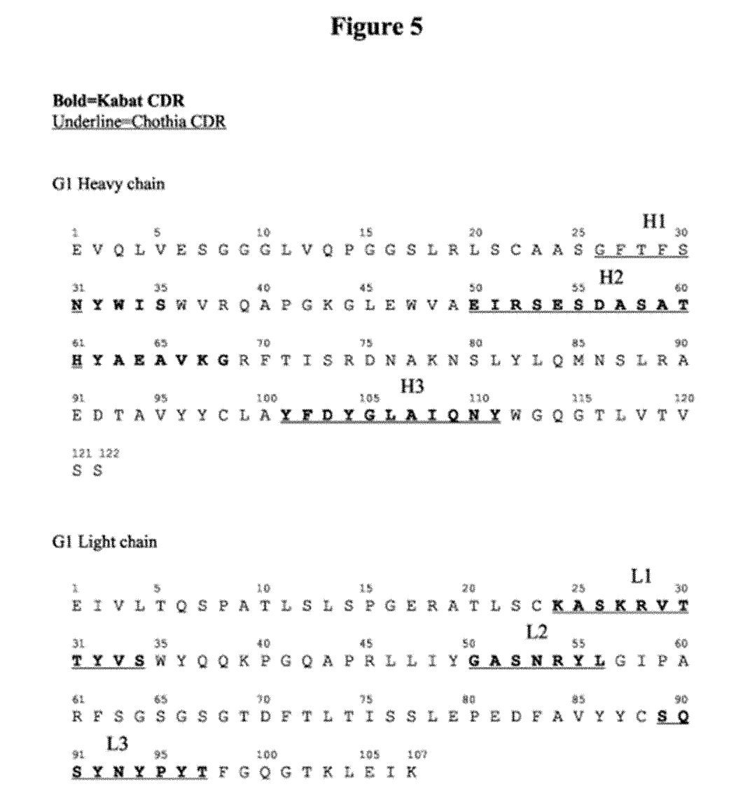

As used herein, the terms “G1,” “antibody G1,” “TEV-48125,” and “fremanezumab” are used interchangeably to refer to an anti-CGRP antagonist antibody produced by expression vectors having deposit numbers of ATCC PTA-6867 and ATCC PTA-6866. The amino acid sequence of the heavy chain and light chain variable regions are shown in FIG. 5. The CDR portions of antibody G1 (including Chothia and Kabat CDRs) are diagrammatically depicted in FIG. 5. The polynucleotides encoding the heavy and light chain variable regions are shown in SEQ ID NO:9 and SEQ ID NO:10. The G1 heavy chain full antibody amino acid sequence is shown in SEQ ID NO:11. The G1 light chain full antibody amino acid sequence is shown in SEQ ID NO:12. The characterization and processes for making antibody G1 (and variants thereof) are described in Examples 1-4 infra, as well as PCT Application No. PCT/IB2006/003181, which is hereby incorporated by reference in its entirety.

The terms “polypeptide”, “oligopeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon an antibody, the polypeptides can occur as single chains or associated chains.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

As used herein, “cluster headaches” (CH) are attacks of severe, strictly unilateral pain which is orbital, supraorbital, temporal, or in any combination of these sites, lasting 15-180 minutes and occurring from once every other day to eight times a day. The pain can be associated with ipsilateral conjunctival injection, lacrimation, nasal congestion, rhinorrhoea, forehead and facial sweating, miosis, ptosis and/or eyelid oedema, and/or with restlessness or agitation, as further described in The International Classification of Headache Disorders, 3^{rd} edition (beta version), Cephalalgia, 33(9): 629-808 (2013).

Skilled practitioners will be readily able to recognize a subject with a cluster headache. For example, diagnostic criteria for a cluster headache can include:
A. At least five attacks fulfilling criteria B-D
B. Severe or very severe unilateral orbital, supraorbital and/or temporal pain lasting 15-180 minutes (when untreated)
C. Either or both of the following:
   1. at least one of the following symptoms or signs, ipsilateral to the headache:
      a) conjunctival injection and/or lacrimation
      b) nasal congestion and/or rhinorrhoea
      c) eyelid oedema
      d) forehead and facial sweating
      e) forehead and facial flushing
      f) sensation of fullness in the ear
      g) miosis and/or ptosis
   2. a sense of restlessness or agitation
D. Attacks have a frequency between one every other day and eight per day for more than half of the time when the disorder is active
E. Not better accounted for by another ICHD-3 diagnosis.

Episodic cluster headaches are cluster headache attacks occurring in periods lasting from seven days to one year, separated by pain-free periods lasting at least one month. Diagnostic criteria can include:

A. Attacks fulfilling criteria described above for cluster headache and occurring in bouts (cluster periods)

B. At least two cluster periods lasting from seven days to one year (when untreated) and separated by pain-free remission periods of ≥1 month.

Cluster periods can last between two weeks and three months.

Chronic cluster headaches are characterized as cluster headache attacks occurring for more than one year without remission, or with remission periods lasting less than one month. Diagnostic criteria can include attacks fulfilling criteria described above for cluster headache, and occurring without a remission period, or with remissions lasting less than one month, for at least one year.

As used herein, “preventing” is an approach to stop CCH or ECH from occurring or existing in a subject, who does not already have CCH or ECH. As used herein, “treatment” is an approach for obtaining beneficial or desired clinical results. For purposes of this invention, beneficial or desired clinical results include, but are not limited to, one or more of the following: improvement in any aspect of a CCH or ECH, including lessening severity, alleviation of pain intensity, and other associated symptoms, reducing frequency of recurrence, increasing the quality of life of those suffering from the CCH or ECH, and decreasing dose of other medications required to treat the CCH or ECH.

“Reducing incidence” of CCH or ECH means any of reducing severity (which can include reducing need for and/or amount of (e.g., exposure to) other drugs and/or therapies generally used for this condition, including, for example, ergotamine, dihydroergotamine, or triptans), duration, and/or frequency (including, for example, delaying or increasing time to next episodic attack in an individual). As is understood by those skilled in the art, individuals may vary in terms of their response to treatment, and, as such, for example, a “method of reducing incidence of CCH or ECH in an individual” reflects administering the anti-CGRP antagonist antibody based on a reasonable expectation that such administration may likely cause such a reduction in incidence in that particular individual.

“Ameliorating” CCH or ECH or one or more symptoms of CCH or ECH means a lessening or improvement of one or more symptoms of CCH or ECH as compared to not administering an anti-CGRP antagonist antibody. “Ameliorating” also includes shortening or reduction in duration of a symptom.

As used herein, “controlling CCH or ECH” refers to maintaining or reducing severity or duration of one or more symptoms of CCH or ECH or frequency of CCH or ECH attacks in an individual (as compared to the level before treatment). For example, the duration or severity of head pain, or frequency of attacks is reduced by at least about any of 10%, 20%, 30%, 40%, 50%, 60%, or 70% in the individual as compared to the level before treatment.

As used herein, a “headache hour” refers to an hour during which a subject experiences headache. Headache hours can be expressed in terms of whole hours (e.g., one headache hour, two headache hours, three headache hours, etc.) or in terms of whole and partial hours (e.g., 0.5 headache hours, 1.2 headache hours, 2.67 headache hours, etc.). One or more headache hours may be described with respect to a particular time interval. For example, “daily headache hours” may refer to the number of headache hours a subject experiences within a day interval (e.g., a 24-hour period). In another example, “weekly headache hours” may refer to the number of headache hours a subject experiences within a week interval (e.g., a 7-day period). As can be appreciated, a week interval may or may not correspond to a calendar week. In another example, “monthly headache hours” may refer to the number of headache hours a subject experiences within a month interval. As can be appreciated, a month interval (e.g., a period of 28, 29, 30, or 31 days) may vary in terms of number of days depending upon the particular month and may or may not correspond to a calendar month. In yet another example, “yearly headache hours” may refer to the number of headache hours a subject experiences within a year interval. As can be appreciated, a year interval (e.g., a period of 365 or 366 days) may vary in terms of number of days depending upon the particular year and may or may not correspond to a calendar year.

As used herein, a “headache day” refers to a day during which a subject experiences headache. Headache days can be expressed in terms of whole days (e.g., one headache day, two headache days, three headache days, etc.) or in terms of whole and partial days (e.g., 0.5 headache days, 1.2 headache days, 2.67 headache days, etc.). One or more headache days may be described with respect to a particular time interval. For example, “weekly headache days” may refer to the number of headache days a subject experiences within a week interval (e.g., a 7-day period). As can be appreciated, a week interval may or may not correspond to a calendar week. In another example, “monthly headache days” may refer to the number of headache days a subject experiences within a month interval. As can be appreciated, a month interval (e.g., a period of 28, 29, 30, or 31 days) may vary in terms of number of days depending upon the particular month and may or may not correspond to a calendar month. In yet another example, “yearly headache days” may refer to the number of headache days a subject experiences within a year interval. As can be appreciated, a year interval (e.g., a period of 365 or 366 days) may vary in terms of number of days depending upon the particular year and may or may not correspond to a calendar year.

As used therein, “delaying” the development of CCH or ECH means to defer, hinder, slow, retard, stabilize, and/or postpone progression of the disease. This delay can be of varying lengths of time, depending on the history of the disease and/or individuals being treated. As is evident to one skilled in the art, a sufficient or significant delay can, in effect, encompass prevention, in that the individual does not develop CCH or ECH. A method that “delays” development of the symptom is a method that reduces probability of developing the symptom in a given time frame and/or reduces extent of the symptoms in a given time frame, when compared to not using the method. Such comparisons are typically based on clinical studies, using a statistically significant number of subjects.

“Development” or “progression” of CCH or ECH means initial manifestations and/or ensuing progression of the disorder. Development of CCH or ECH can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of CCH or ECH includes initial onset and/or recurrence.

As used herein, an “effective dosage” or “effective amount” of drug, compound, or pharmaceutical composition is an amount sufficient to effect beneficial or desired results. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as reducing pain intensity, duration, or frequency of CCH or ECH attack, and decreasing one or more symptoms resulting from CCH or ECH (biochemical, histological and/or behavioral), including its complications and intermediate pathological phenotypes presenting during development of the disease, increasing the quality of life of those suffering from the disease, decreasing the dose of other medications required to treat the disease, enhancing effect of another medication, and/or delaying the progression of the disease of patients. An effective dosage can be administered in one or more administrations. For purposes of this disclosure, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

An “individual” or a “subject” is a mammal, more preferably a human. Mammals also include, but are not limited to, farm animals, sport animals, pets, primates, horses, dogs, cats, mice and rats.

A. Methods for Preventing, Treating, or Reducing CCH or ECH and/or at Least One Secondary Symptom Associated with CCH or ECH

In one aspect, the invention provides methods of preventing, treating, or reducing incidence of CCH or ECH in a subject. In another aspect, the invention provides a method of treating or reducing incidence of at least one secondary symptom associated with CCH or ECH in a subject. In some embodiments, the method comprises administering to the individual an effective amount of an antibody or polypeptides derived from the antibody that modulates the CGRP pathway (e.g., a monoclonal anti-CGRP antagonist antibody).

In another aspect, the invention provides methods for preventing, ameliorating, controlling, reducing incidence of, or delaying the development or progression of CCH or ECH in an individual or symptoms associated with CCH or ECH comprising administering to the individual an effective amount of an antibody that modulates the CGRP pathway or an anti-CGRP antagonist antibody in combination with at least one additional agent useful for preventing, treating, or reducing CCH or ECH.

Such additional agents include, but are not limited to, 5-HT agonists and NSAIDs. For example, the antibody and the at least one additional agent can be concomitantly administered, i.e., they can be given in close enough temporal proximity to allow their individual therapeutic effects to overlap. For example, the amount of 5-HT agonist or NSAID administered in combination with an anti-CGRP antibody should be sufficient to reduce the frequency of CCH or ECH relapse in patients or produce longer lasting efficacy compared to the administration of either one of these agents in the absence of the other.

Additional non-limiting examples of additional agents that may be administered in combination with an anti-CGRP antagonist antibody include one or more of:

(i) an opioid analgesic, e.g., morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine or pentazocine;
(ii) a nonsteroidal antiinflammatory drug (NSAID), e.g., aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, nabumetone, naproxen, oxaprozin, phenylbutazone, piroxicam, sulindac, tolmetin or zomepirac, cyclooxygenase-2 (COX-2) inhibitors, celecoxib; rofecoxib; meloxicam; JTE-522; L-745,337; NS398; or a pharmaceutically acceptable salt thereof;
(iii) a barbiturate sedative, e.g., amobarbital, aprobarbital, butabarbital, butabital, (including butalbital combinations, e.g., butalbital/aspirin/caffeine (Fiorinal®, Actavis) or butalbital/paracetamol/caffeine (Fioricet®, Cardinal Health)) mephobarbital, metharbital, methohexital, pentobarbital, phenobartital, secobarbital, talbutal, theamylal or thiopental or a pharmaceutically acceptable salt thereof;
(iv) a barbiturate analgesic, e.g., butalbital or a pharmaceutically acceptable salt thereof or a composition comprising butalbital.
(v) a benzodiazepine having a sedative action, e.g., chlordiazepoxide, clorazepate, diazepam, flurazepam, lorazepam, oxazepam, temazepam, or triazolam or a pharmaceutically acceptable salt thereof;
(vi) an H₁ antagonist having a sedative action, e.g., diphenhydramine, pyrilamine, promethazine, chlorpheniramine, or chlorcyclizine or a pharmaceutically acceptable salt thereof;
(vii) a sedative such as glutethimide, meprobamate, methaqualone or dichloralphenazone or a pharmaceutically acceptable salt thereof;
(viii) a skeletal muscle relaxant, e.g., baclofen, carisoprodol, chlorzoxazone, cyclobenzaprine, methocarbamol or orphrenadine or a pharmaceutically acceptable salt thereof;
(ix) an NMDA receptor antagonist, e.g., dextromethorphan ((+)-3-hydroxy-N-methylmorphinan) or its metabolite dextrorphan ((+)-3-hydroxy-N-methylmorphinan), ketamine, memantine, pyrroloquinoline quinone or cis-4-(phosphonomethyl)-2-piperidinecarboxylic acid or a pharmaceutically acceptable salt thereof;
(x) an alpha-adrenergic, e.g., doxazosin, tamsulosin, clonidine or 4-amino-6,7-dimethoxy-2-(5-methanesulfonamido-1,2,3,4-tetrahydroisoquinol-2-yl)-5-(2-pyridyl) quinazoline;
(xi) a tricyclic antidepressant, e.g., desipramine, imipramine, amytriptiline or nortriptiline;
(xii) an anticonvulsant, e.g., carbamazepine or valproate;
(xiii) a tachykinin (NK) antagonist, particularly an NK-3, NK-2 or NK-1 antagonist, e.g., (αR,9R)-7-[3,5-bis(trifluoromethyl)benzyl]-8,9,10,11-tetrahydro-9-methyl-5-(4-methylphenyl)-7H-[1,4]diazocino[2,1-g][1,7]naphthridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]methyl]-1,2-dihydro-3H-1,2,4-triazol-3-one (MK-869), lanepitant, dapitant or 3-[[2-methoxy-5-(trifluoromethoxy)phenyl]methylamino]-2-phenyl-piperidine (2S,3S);
(xiv) a muscarinic antagonist, e.g., oxybutin, tolterodine, propiverine, tropsium chloride or darifenacin;
(xv) a COX-2 inhibitor, e.g., celecoxib, rofecoxib or valdecoxib;
(xvi) a non-selective COX inhibitor (preferably with GI protection), e.g., nitroflurbiprofen (HCT-1026);
(xvii) a coal-tar analgesic, in particular paracetamol;
(xviii) a neuroleptic such as droperidol;
(xix) a vanilloid receptor agonist (e.g., resinferatoxin) or antagonist (e.g., capsazepine);
(xx) a beta-adrenergic such as propranolol;
(xxi) a local anaesthetic, such as mexiletine;
(xxii) a corticosteroid, such as dexamethasone;
(xxiii) a serotonin receptor agonist or antagonist;
(xxiv) a cholinergic (nicotinic) analgesic;
(xxv) Tramadol (trade mark);
(xxvi) a PDEV inhibitor, such as sildenafil, vardenafil or taladafil;
(xxvii) an alpha-2-delta ligand such as gabapentin or pregabalin;
(xxviii) a canabinoid; and
(xxix) an antidepressant, such as amitriptyline (Elavil), trazodone (Desyrel), and imipramine (Tofranil) or anticonvulsants such as phenytoin (Dilantin) or carbamazepine (Tegretol).

Those skilled in the art will be able to determine appropriate dosage amounts for particular agents to be used in combination with an anti-CGRP antibody. For example, sumatriptan may be administered in a dosage from about 0.01 to about 300 mg. In some cases, sumatriptan may be administered in a dosage from about 2 mg to about 300 mg, e.g., about 5 mg to about 250 mg, about 5 mg to about 200 mg, about 5 mg to about 100 mg, about 5 mg to about 50 mg, or about 5 mg to about 25 mg. When administered non-parenterally, the typical dosage of sumatriptan is from about 25 to about 100 mg with about 50 mg being generally preferred, e.g., about 45 mg, about 55 mg, or about 60 mg. When sumatriptan is administered parenterally, the preferred dosage is about 6 mg, e.g., about 5 mg, about 7 mg, or about 8 mg. However, these dosages may be varied according to methods standard in the art so that they are optimized for a particular patient or for a particular combination therapy. Further, for example, celecoxib may be administered in an amount of between 50 and 500 mg, e.g., about 50 mg to about 400 mg, about 50 mg to about 300 mg, about 50 mg to about 200 mg, about 50 mg to about 100 mg, about 100 mg to about 400 mg, or about 200 mg to about 300 mg.

In another aspect, the disclosure provides a method of preventing, treating, or reducing incidence of CCH or ECH in a subject comprising administering to the subject a monoclonal antibody (e.g., a monoclonal, anti-CGRP antagonist antibody) that modulates the CGRP pathway. In some embodiments, the amount of the monoclonal antibody administered on each of the plurality of days may be between 0.1 mg-5000 mg, 1 mg-5000 mg, 10 mg-5000 mg, 100 mg-5000 mg, 1000 mg-5000 mg, 0.1 mg-4000 mg, 1 mg-4000 mg, 10 mg-4000 mg, 100 mg-4000 mg, 1000 mg-4000 mg, 0.1 mg-3000 mg, 1 mg-3000 mg, 10 mg-3000 mg, 100 mg-3000 mg, 1000 mg-3000 mg, 0.1 mg-2000 mg, 1 mg-2000 mg, 10 mg-2000 mg, 100 mg-2000 mg, 1000 mg-2000 mg, 0.1 mg-1000 mg, 1 mg-1000 mg, 10 mg-1000 mg, or 100 mg-1000 mg. In some embodiments, the amount is between about 225 mg and about 1000 mg, e.g., about 675 mg or about 900 mg. An exemplary dosing regimen comprises administering an initial antibody dose of about 675 mg subcutaneously, followed by a monthly antibody dose of about 225 mg subcutaneously for about, e.g., about two months, three months, four months, five months, six months, seven months, eight months, nine months, ten months, 11 months or 12 months, or even a period of greater than one year (e.g., 18 months, two years, or three years). In one embodiment, the dosing regimen comprises administering an initial antibody dose of about 900 mg intravenously, followed by a monthly antibody dose of about 225 mg subcutaneously for, e.g., about two months, three months, four months, five months, six months, seven months, eight months, nine months, ten months, 11 months, or 12 months, or even a period of greater than one year (e.g., 18 months, two years, or three years). Yet another dosing regimen comprises administering an initial antibody dose of about 900 mg intravenously in an infusion over about 60 minutes, followed by doses of about 900 mg administered intravenously in an infusion over about 60 minutes every quarter for one year, two years, three years, four years, or five years. However, other dosage regimens may be useful, depending on the pattern of pharmacokinetic decay that the practitioner wishes to achieve. In some embodiments, the initial dose and one or more of the additional doses are administered the same way, e.g., subcutaneously or intravenously. In some embodiments, the one or more additional doses are administered in a different way than the initial dose, e.g., the initial dose may be administered intravenously and the one or more additional doses may be administered subcutaneously.

In another aspect, the disclosure provides a method of preventing, treating, or reducing incidence of CCH or ECH in a subject comprising administering to the subject a single dose of a monoclonal antibody (e.g., a monoclonal, anti-CGRP antagonist antibody) in an amount that modulates the CGRP pathway. In some embodiments, the single dose may be an amount of antibody between 0.1 mg-5000 mg, 1 mg-5000 mg, 10 mg-5000 mg, 100 mg-5000 mg, 1000 mg-5000 mg, 0.1 mg-4000 mg, 1 mg-4000 mg, 10 mg-4000 mg, 100 mg-4000 mg, 1000 mg-4000 mg, 0.1 mg-3000 mg, 1 mg-3000 mg, 10 mg-3000 mg, 100 mg-3000 mg, 1000 mg-3000 mg, 0.1 mg-2000 mg, 1 mg-2000 mg, 10 mg-2000 mg, 100 mg-2000 mg, 1000 mg-2000 mg, 0.1 mg-1000 mg, 1 mg-1000 mg, 10 mg-1000 mg or 100 mg-1000 mg. In some embodiments, the single dose may be an amount of antibody between 225 mg and about 1000 mg, e.g., about 675 mg or about 900 mg.

In another aspect, the disclosure provides a method of preventing, treating, or reducing incidence of CCH or ECH in a subject comprising administering to the subject a monthly dose of a monoclonal antibody (e.g., a monoclonal, anti-CGRP antagonist antibody) in an amount that modulates the CGRP pathway. In some embodiments, the single dose may be an amount of antibody between 0.1 mg-5000 mg, 1 mg-5000 mg, 10 mg-5000 mg, 100 mg-5000 mg, 1000 mg-5000 mg, 0.1 mg-4000 mg, 1 mg-4000 mg, 10 mg-4000 mg, 100 mg-4000 mg, 1000 mg-4000 mg, 0.1 mg-3000 mg, 1 mg-3000 mg, 10 mg-3000 mg, 100 mg-3000 mg, 1000 mg-3000 mg, 0.1 mg-2000 mg, 1 mg-2000 mg, 10 mg-2000 mg, 100 mg-2000 mg, 1000 mg-2000 mg, 0.1 mg-1000 mg, 1 mg-1000 mg, 10 mg-1000 mg or 100 mg-1000 mg. In some embodiments, the monthly dose may be an amount of antibody between about 225 mg and about 1000 mg, e.g., about 675 mg or about 900 mg. An exemplary dosing regimen comprises administering an initial antibody dose of about 675 mg subcutaneously, followed by a monthly antibody dose of about 225 mg subcutaneously for, e.g., about two months, three months, four months, five months, six months, seven months, eight months, nine months, ten months, 11 months, or 12 months, or even a period of greater than one year (e.g., 18 months, two years, or three years). In one embodiment, the dosing regimen comprises administering an initial antibody dose of about 900 mg intravenously, followed by a monthly antibody dose of about 225 mg subcutaneously for, e.g., about two months, three months, four months, five months, six months, seven months, eight months, nine months, ten months, 11 months, or 12 months, or even a period of greater than one year (e.g., 18 months, two years, or three years). Yet another dosing regimen comprises administering an initial antibody dose of about 900 mg intravenously in an infusion over about 60 minutes, followed by doses of about 900 mg administered intravenously in an infusion over about 60 minutes every quarter for one year, two years, three years, four years, or five years. However, other dosage regimens may be useful, depending on the pattern of pharmacokinetic decay that the practitioner wishes to achieve. In some embodiments, the initial dose and one or more of the additional doses are administered the same way, e.g., subcutaneously or intravenously. In some embodiments, the one or more additional doses are administered in a different way than the initial dose, e.g., the initial dose may be administered intravenously and the one or more additional doses may be administered subcutaneously.

In another aspect, the disclosure provides a method of decreasing a number of monthly headache hours experienced by a subject, comprising administering to the subject an amount of a monoclonal antibody (e.g., a monoclonal, anti-CGRP antagonist antibody) that modulates the CGRP pathway. In some embodiments, the monoclonal antibody can be in an amount effective to decrease the number of monthly headache hours by at least 0.1, 1, 5, 10, 15, 20, 25, 30, 35, 40, 45, 50, 55, 60, 65, 70, 75, 80, 85, 90, 95, 100 or more headache hours after a single dose, monthly dose, or quarterly dose. In some embodiments, the monoclonal antibody can be in an amount effective to decrease the number of monthly headache hours by at least 20 headache hours after a single dose, monthly dose, or quarterly dose. In some embodiments, the monoclonal antibody can be in an amount effective to decrease the number of monthly headache hours by at least 40, 45, 50, 55, 60, 65, 70, 75, 80, 85, 90, 95, 100, 105, 110, 115, 120, 125, or more headache hours. In some embodiments, the monoclonal antibody can be in an amount effective to decrease the number of monthly headache hours by at least 0.1%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or more after a single dose. In some embodiments, the monoclonal can be in an amount effective to decrease the number of monthly headache hours by at least 15%, 20%, 25%, 30%, 35%, 40%, 45%, 50%, 55%, 60%, 65%, 70%, 75%, 80%, 85%, 90%, 95%, 99%, or more after a single dose, monthly dose, or quarterly dose.

In another aspect, the disclosure provides a method of decreasing a number of monthly headache days experienced by a subject, comprising administering to the subject an amount of a monoclonal antibody (e.g., a monoclonal, anti-CGRP antagonist antibody) that modulates the CGRP pathway. In some embodiments, the monoclonal antibody can be in an amount effective to decrease the number of monthly headache days by at least 3, 4, 5, 6, 7, 8, 9, 10, 11, 12, 13, 14, 15, 16, 17, 18, 19, 20, or more headache days after a single dose. In some embodiments, the monoclonal antibody can be in an amount effective to decrease the number of monthly headache days by at least 3, 4, 5, 6, 7, 8, 9, 10, 11, 12, 13, 14, 15, 16, 17, 18, 19, 20, or more headache days after a monthly dose or quarterly dose. In some embodiments, the monoclonal antibody can be in an amount effective to decrease the number of monthly headache days by at least 0.1%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or more after a single dose, monthly dose, or quarterly dose.

In another aspect, the disclosure provides a method of decreasing use of an anti-headache medication in a subject, comprising administering to the subject a monoclonal antibody (e.g., a monoclonal anti-CGRP antagonist antibody) that modulates the CGRP pathway. In some embodiments, the monoclonal antibody can be in an amount effective to decrease daily, monthly, quarterly, and/or yearly use of the anti-headache medication by the subject by at least 0.1%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or more. In some embodiments, the monoclonal antibody can be in an amount effective to decrease monthly use of the anti-headache medication by the subject by at least 15%, 20%, 25%, 30%, 35%, 40%, 45%, 50%, 55%, 60%, 65%, 70%, 75%, 80%, 85%, 90%, 95%, 99%, or more. The anti-headache medication can be any type of anti-headache medication described herein. Non-limiting examples of anti-headache medications include, for example, 5-HT1 agonists (and agonists acting at other 5-HT1 sites), triptans (e.g., sumatriptan, zolmitriptan, naratriptan, rizatriptan, eletriptan, almotriptan, afrovatriptan), ergot alkaloids (e.g., ergotamine tartrate, ergonovine maleate, and ergoloid mesylates (e.g., dihydroergocornine, dihydroergocristine, dihydroergocryptine, and dihydroergotamine mesylate (DHE 45)) and non-steroidal anti-inflammatory drugs (NSAIDs) (e.g., aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, nabumetone, naproxen, oxaprozin, phenylbutazone, piroxicam, sulindac, tolmetin or zomepirac, cyclooxygenase-2 (COX-2) inhibitors, celecoxib; rofecoxib; meloxicam; JTE-522; L-745,337; NS398; or a pharmaceutically acceptable salt thereof), opiates (e.g., oxycodone), and β-adrenergic antagonists (e.g., propranolol).

In another aspect, the disclosure provides a method of decreasing the weekly average number of days of use of a cluster-specific acute headache medication in a subject having cluster headache (ECH or CCH), comprising administering to the subject a monoclonal antibody (e.g., a monoclonal anti-CGRP antagonist antibody) that modulates the CGRP pathway. In some embodiments, the monoclonal antibody can be in an amount effective to decrease the weekly average number of days of use of the acute headache medication by 1, 2, 3, 4, 5, 6, or 7 days after a single dose. In some embodiments, the monoclonal antibody can be in an amount effective to decrease the weekly average number of days of use of the acute headache medication by 1, 2, 3, 4, 5, 6, or 7 days after a monthly dose or quarterly dose. In some embodiments, the cluster-specific acute headache medication is a triptan or ergot compound.

In another aspect, the disclosure provides a method of decreasing the weekly average number of days of use of oxygen to treat a subject having cluster headache (ECH or CCH), comprising administering to the subject a monoclonal antibody (e.g., a monoclonal anti-CGRP antagonist antibody) that modulates the CGRP pathway. In some embodiments, the monoclonal antibody can be in an amount effective to decrease the weekly average number of days of use of the oxygen by 1, 2, 3, 4, 5, 6, or 7 days after a single dose. In some embodiments, the monoclonal antibody can be in an amount effective to decrease the weekly average number of days of use of the oxygen by 1, 2, 3, 4, 5, 6, or 7 days after a monthly dose or quarterly dose.

In another aspect, the disclosure provides a method of improving the health-related quality of life of a subject having cluster headache, comprising administering to the subject a monoclonal antibody (e.g., a monoclonal anti-CGRP antagonist antibody) that modulates the CGRP pathway. In some embodiments, changes in health-related quality of life are self-reported by the subject. In some embodiments, changes in the quality of life of a subject are measured using a Patient-Perceived Satisfactory Improvement (PPSI) or the Patient Global Impression of Change (PGIC) scale. The PPSI and PGIC assessments and various versions thereof, are known in the art.

With respect to all methods described herein, references to antibodies (e.g., monoclonal antibodies that modulate the CGRP pathway, anti-CGRP antagonist antibodies, monoclonal anti-CGRP antagonist antibodies) also include compositions comprising one or more of these agents. Accordingly, such a composition may be used according to a method referring to an antibody described herein. These compositions may further comprise suitable excipients, such as pharmaceutically acceptable excipients as described elsewhere herein. The present invention can be used alone or in combination with other conventional methods of treatment.

An antibody described herein (e.g., a monoclonal antibody, an anti-CGRP antagonist antibody, a monoclonal anti-CGRP antagonist antibody) can be administered to an individual or subject in any therapeutic dose, via any suitable route and in any suitable formulation. It should be apparent to a person skilled in the art that the examples described herein are not intended to be limiting but to be illustrative of the techniques available. Accordingly, in some embodiments, an antibody described herein can be administered to a subject in accord with known methods, such as intravenous administration, e.g., as a bolus or by continuous infusion over a period of time, e.g., about 10 minutes, about 20 minutes, about 30 minutes, about 40 minutes, about 50 minutes, about 60 minutes, about 90 minutes, about 120 minutes, about 180 minutes, or about 240 minutes. The antibody described herein can also be administered to the subject by subcutaneous, intramuscular, intraperitoneal, intracerebrospinal, intra-articular, sublingually, intra-arterial, intrasynovial, via insufflation, intrathecal, oral, inhalation, intranasal (e.g., with or without inhalation), buccal, rectal, transdermal, intracardiac, intraosseous, intradermal, transmucosal, vaginal, intravitreal, peri-articular, local, epicutaneous, or topical routes. Administration can be systemic, e.g., intravenous administration, or localized. Commercially available nebulizers for liquid formulations, including jet nebulizers and ultrasonic nebulizers are useful for administration. Liquid formulations can be directly nebulized and lyophilized powder can be nebulized after reconstitution. Alternatively, an antibody described herein can be aerosolized using a fluorocarbon formulation and a metered dose inhaler, or inhaled as a lyophilized and milled powder.

In some embodiments, an antibody described herein can be administered via site-specific or targeted local delivery techniques. Examples of site-specific or targeted local delivery techniques include various implantable depot sources of the antibody or local delivery catheters, such as infusion catheters, an indwelling catheter, or a needle catheter, synthetic grafts, adventitial wraps, shunts and stents or other implantable devices, site specific carriers, direct injection, or direct application. See e.g., PCT Publication No. WO 00/53211 and U.S. Pat. No. 5,981,568, which are hereby incorporated by reference in their entireties.

Various formulations of an antibody described herein may be used for administration. In some embodiments, an antibody may be administered neat. In some embodiments, antibody and a pharmaceutically acceptable excipient may be in various formulations. Pharmaceutically acceptable excipients are known in the art, and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington, The Science and Practice of Pharmacy 20th Ed. Mack Publishing (2000).

In some embodiments, these agents, including antibodies described herein, may be formulated for administration by injection (e.g., intravenously, subcutaneously, intraperitoneally, intramuscularly, etc.). Accordingly, these agents can be combined with pharmaceutically acceptable vehicles such as saline, Ringer's solution, dextrose solution, and the like. The particular dosage regimen, i.e., dose, timing and repetition, will depend on the particular individual and that individual's medical history.

In some embodiments, these agents, including antibodies described herein, may be formulated for peripheral administration. Such formulations can be administered peripherally via any suitable peripheral route, including intravenously and subcutaneously. An agent prepared for peripheral administration can include a substance, medicament, and/or antibody that is not delivered centrally, spinally, intrathecally, or directly into the CNS. Non-limiting examples of peripheral administration routes include a route which is oral, sublingual, buccal, topical, rectal, via inhalation, transdermal, subcutaneous, intravenous, intra-arterial, intramuscular, intracardiac, intraosseous, intradermal, intraperitoneal, transmucosal, vaginal, intravitreal, intra-articular, peri-articular, local, or epicutaneous.

Therapeutic formulations of the antibodies used in accordance with the present disclosure can be prepared for storage and/or use by mixing an antibody having the desired degree of purity with optional pharmaceutically acceptable carriers, excipients or stabilizers (Remington, The Science and Practice of Pharmacy 20th Ed. Mack Publishing (2000)), and can in some cases be in the form of lyophilized formulations or aqueous solutions. Acceptable carriers, excipients, or stabilizers are nontoxic to recipients at the dosages and concentrations employed. A therapeutic formulation of an antibody may comprise one or more pharmaceutically acceptable carriers, excipients or stabilizes with non-limiting examples of such species that include buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben;

catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids (e.g., at concentrations of 0.1 mM to 100 mM, 0.1 mM to 1 mM, 0.01 mM to 50 mM, 1 mM to 50 mM, 1 mM to 30 mM, 1 mM to 20 mM, 10 mM to 25 mM) such as glycine, glutamine, methionine, asparagine, histidine, arginine, or lysine; monosaccharides, disaccharides, and other carbohydrates including glucose, mannose, or dextrins; chelating agents (e.g., at concentrations of 0.001 mg/mL to 1 mg/mL, 0.001 mg/mL to 1 mg/mL, 0.001 mg/mL to 0.1 mg/mL, 0.001 mg/mL to 0.01 mg/mL, 0.01 mg/mL to 0.1 mg/mL) such as EDTA (e.g., disodium EDTA dihydrate); sugars (e.g., at concentrations of 1 mg/mL to 500 mg/mL, 10 mg/mL to 200 mg/mL, 10 mg/mL to 100 mg/mL, 50 mg/mL to 150 mg/mL) such as sucrose, mannitol, trehalose or sorbitol; salt-forming counter-ions such as sodium; metal complexes (e.g., Zn-protein complexes); and/or non-ionic surfactants (e.g., at concentrations of 0.01 mg/mL to 10 mg/mL, 0.01 mg/mL to 1 mg/mL, 0.1 mg/mL to 1 mg/mL, 0.01 mg/mL to 0.5 mg/mL) such as TWEEN™ (e.g., polysorbate (e.g., polysorbate 20, polysorbate 40, polysorbate 60, polysorbate 80)), PLURONICS™ or polyethylene glycol (PEG).

An antibody formulation may be characterized in terms of any of a variety of physical properties. For example, a liquid antibody formulation may have any suitable pH for therapeutic efficacy, safety and storage. For example, the pH of a liquid antibody formulation may be from pH 4 to about pH 9, from about pH 5 to about pH 8, from about pH 5 to about pH 7 or from about pH 6 to about pH 8. In some embodiments, a liquid antibody formulation may have a pH of about 3.0, 3.5, 4.0, 4.5, 5.0, 5.5, 6.0, 6.5, 7.0, 7.5, 8.0, 8.5, 9.0, 9.5, or about 10 or higher or lower.

In another example, a liquid antibody formulation may have any suitable viscosity for therapeutic efficacy, safety and storage. For example, the viscosity of a liquid antibody formulation may be from about 0.5 centipoise (cP) to about 100 cP, about 1 cP to about 50 cP, about 1 cP to about 20 cP, about 1 cP to about 15 cP, or about 5 cP to about 15 cP at 25° C. In some embodiments, a liquid antibody formulation may have a viscosity of about 0.5 cP, 1 cP, 1.2 cP, 1.4 cP, 1.6 cP, 1.8 cP, 2.0 cP, 2.2 cP, 2.4 cP, 2.6 cP, 2.8 cP, 3.0 cP, 3.2 cP, 3.4 cP, 3.6 cP, 3.8 cP, 4.0 cP, 4.2 cP, 4.4 cP, 4.6 cP, 4.8 cP, 5.0 cP, 5.2 cP, 5.4 cP, 5.6 cP, 5.8 cP, 6.0 cP, 6.2 cP, 6.4 cP, 6.6 cP, 6.8 cP, 7.0 cP, 7.2 cP, 7.4 cP, 7.6 cP, 7.8 cP, 8.0 cP, 8.2 cP, 8.4 cP, 8.6 cP, 8.8 cP, 9.0 cP, 9.2 cP, 9.4 cP, 9.6 cP, 9.8 cP, 10.0 cP, 10.2 cP, 10.4 cP, 10.6 cP, 10.8 cP, 11.0 cP, 11.2 cP, 11.4 cP, 11.6 cP, 11.8 cP, 12.0 cP, 12.2 cP, 12.4 cP, 12.6 cP, 12.8 cP, 13.0 cP, 13.2 cP, 13.4 cP, 13.6 cP, 13.8 cP, 14.0 cP, 14.2 cP, 14.4 cP, 14.6 cP, 14.8 cP, or about 15.0 cP at 25° C. or the viscosity may be higher or lower.

In another example, a liquid antibody formulation may have any suitable conductivity for therapeutic efficacy, safety and storage. For example, the conductivity of a liquid antibody formulation may be from about 0.1 millisiemens per centimeter (mS/cm) to about 15 mS/cm, 0.1 mS/cm to 10 mS/cm, 0.1 mS/cm to 5 mS/cm, 0.1 mS/cm to 2 mS/cm or 0.1 mS/cm to 1.5 mS/cm. In some embodiments, a liquid antibody formulation may have a conductivity of 0.19 mS/cm, 0.59 mS/cm, 1.09 mS/cm, 1.19 mS/cm, 1.29 mS/cm, 1.39 mS/cm, 1.49 mS/cm, 1.59 mS/cm, 1.69 mS/cm, 1.79 mS/cm, 1.89 mS/cm, 1.99 mS/cm, 2.09 mS/cm, 2.19 mS/cm, 2.29 mS/cm, 2.39 mS/cm, 2.49 mS/cm, 2.59 mS/cm, 2.69 mS/cm, 2.79 mS/cm, 2.89 mS/cm, 2.99 mS/cm, 3.09 mS/cm, 3.19 mS/cm, 3.29 mS/cm, 3.39 mS/cm, 3.49 mS/cm, 3.59 mS/cm, 3.69 mS/cm, 3.79 mS/cm, 3.89 mS/cm, 3.99 mS/cm, 4.09 mS/cm, 4.19 mS/cm, 4.29 mS/cm, 4.39 mS/cm, 4.49 mS/cm, 4.59 mS/cm, 4.69 mS/cm, 4.79 mS/cm, 4.89 mS/cm, 4.99 mS/cm, 5.09 mS/cm, 6.09 mS/CM, 6.59 mS/cm, 7.09 mS/cm, 7.59 mS/cm, 8.09 mS/cm, 8.59 mS/cm, 9.09 mS/cm, 9.59 mS/cm, 10.09 mS/cm, 10.59 mS/cm, 11.09 mS/cm, 11.59 mS/cm, 12.09 mS/cm, 12.59 mS/cm, 13.09 mS/cm, 13.59 mS/cm, 14.09 mS/cm, 14.59 mS/cm, or about 15.09 mS/cm or the conductivity may be higher or lower.

In another example, a liquid antibody formulation may have any suitable osmolality for therapeutic efficacy, safety, and storage. For example, the osmolality of a liquid antibody formulation may be from about 50 milliosmole per kilogram (mOsm/kg) to about 5000 mOsm/kg, about 50 mOsm/kg to about 2000 mOsm/kg, about 50 mOsm/kg to about 1000 mOsm/kg, about 50 mOsm/kg to about 750 mOsm/kg, or about 50 mOsm/kg to about 500 mOsm/kg. In some embodiments, a liquid antibody formulation may have an osmolality of about 50 mOsm/kg, 60 mOsm/kg, 70 mOsm/kg, 80 mOsm/kg, 90 mOsm/kg, 100 mOsm/kg 120 mOsm/kg, 140 mOsm/kg, 160 mOsm/kg, 180 mOsm/kg, 200 mOsm/kg, 220 mOsm/kg, 240 mOsm/kg, 260 mOsm/kg, 280 mOsm/kg, 300 mOsm/kg, 320 mOsm/kg, 340 mOsm/kg, 360 mOsm/kg, 380 mOsm/kg, 400 mOsm/kg, 420 mOsm/kg, 440 mOsm/kg, 460 mOsm/kg, 480 mOsm/kg, 500 mOsm/kg, 520 mOsm/kg, 540 mOsm/kg, 560 mOsm/kg, 580 mOsm/kg, 600 mOsm/kg, 620 mOsm/kg, 640 mOsm/kg, 660 mOsm/kg, 680 mOsm/kg, 700 mOsm/kg, 720 mOsm/kg, 740 mOsm/kg, 760 mOsm/kg, 780 mOsm/kg, 800 mOsm/kg, 820 mOsm/kg, 840 mOsm/kg, 860 mOsm/kg, 880 mOsm/kg, 900 mOsm/kg, 920 mOsm/kg, 940 mOsm/kg, 960 mOsm/kg, 980 mOsm/kg, 1000 mOsm/kg, 1050 mOsm/kg, 1100 mOsm/kg, 1150 mOsm/kg, 1200 mOsm/kg, 1250 mOsm/kg, 1300 mOsm/kg, 1350 mOsm/kg, 1400 mOsm/kg, 1450 mOsm/kg, about 1500 mOsm/kg, or the osmolality may be higher or lower.

Liposomes containing antibody can be prepared by methods known in the art, such as described in Epstein, et al., Proc. Natl. Acad. Sci. USA 82:3688 (1985); Hwang, et al., Proc. Natl Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy 20th Ed. Mack Publishing (2000).

Sustained-release preparations may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or ‘poly(v nylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The formulations to be used for in vivo administration should generally be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

The compositions according to the present invention may be in unit dosage forms such as tablets, pills, capsules, powders, granules, solutions or suspensions, or suppositories, for oral, parenteral or rectal administration, or administration by inhalation or insufflation. In some cases, a unit dosage form may be supplied in a prefilled receptacle (e.g., a prefilled syringe) useful in administering the unit dosage to a subject.

In some embodiments, a formulation comprising an antibody (e.g., monoclonal antibody that modulates the CGRP pathway, anti-CGRP antagonist antibody, monoclonal anti-CGRP antagonist antibody) described herein may be prepared for any suitable route of administration with an antibody amount ranging from about 0.1 mg to about 3000 mg, about 1 mg to about 1000 mg, about 100 mg to about 1000 mg, or about 100 mg to about 500 mg, about 200 mg to about 800 mg, about 500 mg to about 1500 mg, about 1500 mg to about 2500 mg, or about 2000 mg to about 3000 mg. In some cases, a formulation comprising an antibody (e.g., monoclonal antibody that modulates the CGRP pathway, anti-CGRP antagonist antibody, monoclonal anti-CGRP antagonist antibody) described herein may comprise an antibody amount of, at most, or at least about 0.1 mg, 1 mg, 100 mg, 1 mg, 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1100 mg, 1200 mg, 1300 mg, 1400 mg, 1500 mg, 1600 mg, 1700 mg, 1800 mg, 1900 mg, 2000 mg, or about 3000 mg.

In some embodiments, a liquid formulation comprising an antibody (e.g., monoclonal antibody that modulates the CGRP pathway, anti-CGRP antagonist antibody, monoclonal anti-CGRP antagonist antibody) described herein may be prepared for any suitable route of administration with an antibody concentration ranging from about 0.1 mg/mL to about 500 mg/mL, about 0.1 mg/mL to about 375 mg/mL, about 0.1 mg/mL to about 250 mg/mL, about 0.1 to about 175 mg/mL, about 0.1 to 100 mg/mL, about 1 mg/mL to about 500 mg/mL, about 1 mg/mL to about 375 mg/mL, about 1 mg/mL to about 300 mg/mL, about 1 mg/mL to 250 mg/mL, about 1 mg/mL to 200 mg/mL, about 1 mg/mL to 150 mg/mL, about 1 mg/mL to about 100 mg/mL, about 10 mg/mL to 500 mg/mL, about 10 mg/mL to about 375 mg/mL, about 10 mg/mL to 250 mg/mL, about 10 mg/mL to about 150 mg/mL, about 10 mg/mL to 100 mg/mL, about 100 mg/mL to 500 mg/mL, about 100 mg/mL to 450 mg/mL, about 100 mg/mL to 400 mg/mL, about 100 mg/mL to about 350 mg/mL, about 100 mg/mL to about 300 mg/mL, about 100 mg/mL to about 250 mg/mL, 100 mg/mL to 200 mg/mL, or about 100 mg/mL to about 150 mg/mL. In some embodiments, a liquid formulation may comprise an antibody described herein at a concentration of, of at most, of at least, or less than about 0.1, 0.5, 1, 5, 10, 15 20, 25, 30, 35, 40, 45, 50, 55, 60, 65, 70, 75, 80, 85, 90, 95, 100, 105 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or about 500 mg/mL.

An antibody formulation may comprise one or more components including the antibody and other species described elsewhere herein. The antibody and other components may be in any suitable amount and/or any suitable concentration for therapeutic efficacy of the antibody, safety and storage. In one example, an antibody formulation may be a solution comprising about 51.4 mg/mL antibody (e.g., antibody G1, another anti-CGRP antagonist antibody, or a monoclonal antibody that modulates the CGRP pathway), 16-20 mM histidine, 0.1 mg/mL methionine, 84 mg/mL trehalose dihydrate, 0.05 mg/mL disodium EDTA dihydrate, and 0.2 mg/mL polysorbate 80.

In another example, an antibody formulation may comprise about 200 mg/mL antibody (e.g., antibody G1, another anti-CGRP antagonist antibody, or a monoclonal antibody that modulates the CGRP pathway), 15 mM arginine, 78 mg/mL sucrose, 0.3 mg/mL EDTA, and 0.1 mg/mL polysorbate 80.

In another example, an antibody formulation may comprise about 175 mg/mL antibody (e.g., antibody G1, another anti-CGRP antagonist antibody, or a monoclonal antibody that modulates the CGRP pathway), 20 mM glycine, 88 mg/mL trehalose dihydrate, 0.015 mg/mL EDTA, and 0.25 mg/mL polysorbate 80.

In another example, an antibody formulation may comprise about 225 mg/mL antibody (e.g., antibody G1, another anti-CGRP antagonist antibody, or a monoclonal antibody that modulates the CGRP pathway), 23 mM asparagine, 84 mg/mL sorbitol, 0.1 mg/mL EDTA, and 0.15 mg/mL polysorbate 60.

In another example, an antibody formulation may comprise about 150 mg/mL antibody (e.g., antibody G1, another anti-CGRP antagonist antibody, or a monoclonal antibody that modulates the CGRP pathway), 17 mM asparagine, 74 mg/mL mannitol, 0.025 mg/mL EDTA, and 0.2 mg/mL polysorbate 80.

In another example, an antibody formulation may comprise about 100 mg/mL antibody (e.g., antibody G1, another anti-CGRP antagonist antibody, or a monoclonal antibody that modulates the CGRP pathway), 16 mM arginine, 87 mg/mL mannitol, 0.025 mg/mL EDTA, and 0.15 mg/mL polysorbate 20.

In another example, an antibody formulation may comprise about 250 mg/mL antibody (e.g., antibody G1, another anti-CGRP antagonist antibody, or a monoclonal antibody that modulates the CGRP pathway), 25 mM histidine, 74 mg/mL mannitol, 0.025 mg/mL EDTA, and 0.25 mg/mL polysorbate 20.

In another example, an antibody formulation may comprise about 50 mg/mL antibody (e.g., antibody G1, another anti-CGRP antagonist antibody, or a monoclonal antibody that modulates the CGRP pathway), 19 mM arginine, 84 mg/mL sucrose, 0.05 mg/mL EDTA, and 0.3 mg/mL polysorbate 80.

In another example, an antibody formulation may comprise about 125 mg/mL antibody (e.g., antibody G1, another anti-CGRP antagonist antibody, or a monoclonal antibody that modulates the CGRP pathway), 22 mM glycine, 79 mg/mL trehalose dihydrate, 0.15 mg/mL EDTA, and 0.15 mg/mL polysorbate 80.

In another example, an antibody formulation may be a solution comprising about 175 mg/mL antibody (e.g., antibody G1, another anti-CGRP antagonist antibody, or a monoclonal antibody that modulates the CGRP pathway), 20 mM histidine, 0.1 mg/mL methionine, 84 mg/mL trehalose dihydrate, 0.05 mg/mL disodium EDTA dihydrate, and 0.2 mg/mL polysorbate 80.

In another example, an antibody formulation may comprise about 200 mg/mL antibody (e.g., antibody G1, another anti-CGRP antagonist antibody, or a monoclonal antibody that modulates the CGRP pathway), 30 mM arginine, 78 mg/mL sucrose, 0.3 mg/mL EDTA, and 0.1 mg/mL polysorbate 80.

In another example, an antibody formulation may comprise about 175 mg/mL antibody (e.g., antibody G1, another anti-CGRP antagonist antibody, or a monoclonal antibody that modulates the CGRP pathway), 20 mM glycine, 88 mg/mL trehalose dihydrate, 0.015 mg/mL EDTA, and 0.15 mg/mL polysorbate 80.

In another example, an antibody formulation may comprise about 150 mg/mL antibody (e.g., antibody G1, another anti-CGRP antagonist antibody, or a monoclonal antibody that modulates the CGRP pathway), 20 mM histidine, 84 mg/mL sucrose, 0.05 mg/mL EDTA, and 0.2 mg/mL polysorbate 80.

In another example, an antibody formulation may comprise about 225 mg/mL antibody (e.g., antibody G1, another anti-CGRP antagonist antibody, or a monoclonal antibody that modulates the CGRP pathway), 23 mM histidine, 84 mg/mL sorbitol, 0.1 mg/mL EDTA, and 0.15 mg/mL polysorbate 60.

In another example, an antibody formulation may comprise about 150 mg/mL antibody (e.g., antibody G1, another anti-CGRP antagonist antibody, or a monoclonal antibody that modulates the CGRP pathway), 17 mM asparagine, 74 mg/mL mannitol, 0.3 mg/mL EDTA, and 0.2 mg/mL polysorbate 80.

In another example, an antibody formulation may comprise about 100 mg/mL antibody (e.g., antibody G1, another anti-CGRP antagonist antibody, or a monoclonal antibody that modulates the CGRP pathway), 16 mM arginine, 87 mg/mL mannitol, 0.025 mg/mL EDTA, and 0.25 mg/mL polysorbate 20.

In another example, an antibody formulation may comprise about 250 mg/mL antibody (e.g., antibody G1, another anti-CGRP antagonist antibody, or a monoclonal antibody that modulates the CGRP pathway), 25 mM histidine, 89 mg/mL mannitol, 0.025 mg/mL EDTA, and 0.25 mg/mL polysorbate 20.

In another example, an antibody formulation may comprise 125 mg/mL antibody (e.g., antibody G1, another anti-CGRP antagonist antibody, or a monoclonal antibody that modulates the CGRP pathway), 29 mM arginine, 84 mg/mL sucrose, 0.05 mg/mL EDTA, and 0.3 mg/mL polysorbate 80.

In another example, an antibody formulation may comprise 150 mg/mL antibody (e.g., antibody G1, another anti-CGRP antagonist antibody, or a monoclonal antibody that modulates the CGRP pathway), 25 mM asparagine, 84 mg/mL mannitol, 0.05 mg/mL EDTA, and 0.2 mg/mL polysorbate 80.

In another example, an antibody formulation may comprise 145 mg/mL antibody (e.g., antibody G1, another anti-CGRP antagonist antibody, or a monoclonal antibody that modulates the CGRP pathway), 22 mM histidine, 72 mg/mL trehalose dihydrate, 0.05 mg/mL EDTA, and 0.1 mg/mL polysorbate 80.

An antibody described herein can be administered using any suitable method, including by injection (e.g., intravenously, subcutaneously, intraperitoneally, intramuscularly, etc.). Antibodies can also be administered via inhalation, as described herein. In some cases, an antibody may be administered nasally with or without inhalation. Generally, for administration of an antibody described herein, an initial candidate dosage can be about 2 mg/kg. For the purpose of the present invention, a typical daily dosage might range from about any of 3 μg/kg to 30 μg/kg to 300 μg/kg to 3 mg/kg, to 30 mg/kg to 100 mg/kg or more, depending on the factors mentioned above. For example, dosage of about 1 mg/kg, about 2.5 mg/kg, about 5 mg/kg, about 10 mg/kg, about 25 mg/kg, and about 30 mg/kg may be used. For repeated administrations over several days or longer, depending on the condition, the treatment is sustained until a desired suppression of symptoms occurs or until sufficient therapeutic levels are achieved, for example, to reduce pain. An exemplary dosing regimen comprises administering an initial or starting dose of about 8.5 mg/kg, or about 10 mg/kg, followed by a maintenance dose of about 2.8 mg/kg of an antibody, or followed by a maintenance dose of about 2.8 mg/kg every other week. Another exemplary dosing regimen comprises administering a dose of about 100 mg, 125 mg, 150 mg, 200 mg, 225 mg, 250 mg, 275 mg, 300 mg, 350 mg, 400 mg, 450 mg, 500 mg, 550 mg, 600 mg, about 675 mg, or about 900 mg to a subject once per month (e.g., approximately every 28 days) intravenously in an infusion over about one hour, or subcutaneously. Another exemplary dosing regimen comprises administering an initial or starting antibody dose of about 675 mg subcutaneously, followed by a monthly antibody dose of about 225 mg subcutaneously for, e.g., about two months, three months, four months, five months, six months, seven months, eight months, nine months, ten months, 11 months, or 12 months, or even a period of greater than one year (e.g., 18 months, two years, or three years). Another exemplary dosing regimen comprises administering an initial antibody dose of about 900 mg intravenously, followed by a monthly antibody dose of about 225 mg subcutaneously for, e.g., about two months, three months, four months, five months, six months, seven months, eight months, nine months, ten months, 11 months, or 12 months, or even a period of greater than one year (e.g., 18 months, two years, or three years). Yet another dosing regimen comprises administering an initial dose of about 900 mg intravenously in an infusion over about 60 minutes, followed by doses of about 900 mg administered intravenously in an infusion over about 60 minutes every quarter for one year, two years, three years, four years, or five years. However, other dosage regimens may be useful, depending on the pattern of pharmacokinetic decay that the practitioner wishes to achieve. For example, in some embodiments, dosing from about one to about four times a week is contemplated. The progress of this therapy is easily monitored by conventional techniques and assays. The dosing regimen (including the CGRP antagonist(s) used) can vary over time.

In some embodiments, the dose or amount of an antibody (e.g., monoclonal antibody that modulates the CGRP pathway, anti-CGRP antagonist antibody, monoclonal anti-CGRP antagonist antibody) described herein and administered to a subject may range from about 0.1 μg to about 3000 mg, 1 mg to 1000 mg, 100 mg to 1000 mg, 100 mg to 500 mg, 0.1 mg to 5000 mg, 1 mg to 4000 mg, 250 mg to 1000 mg, 500 mg to 1000 mg, 100 mg to 900 mg, 400 mg to 900 mg, 10 mg to 3000 mg, 10 mg to 2000 mg, 100 mg to 2000 mg, 150 mg to 2000 mg, 200 mg to 2000 mg, 250 mg to 2000 mg, 300 mg to 2000 mg, 350 mg to 2000 mg, 400 mg to 2000 mg, 450 mg to 2000 mg, 500 mg to 2000 mg, 550 mg to 2000 mg, 600 mg to 2000 mg, 650 mg to 2000 mg, 700 mg to 2000 mg, 750 mg to 2000 mg, 800 mg to 2000 mg, 850 mg to 2000 mg, 900 mg to 2000 mg, 950 mg to 2000 mg, or 1000 mg to 2000 mg. In some embodiments, the dose or amount of an antibody described herein and administered to a subject may be, may be at most, may be less than, or may be at least about 0.1 μg, 1 μg, 100 μg, 1 mg, 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1100 mg, 1200 mg, 1300 mg, 1400 mg, 1500 mg, 1600 mg, 1700 mg, 1800 mg, 1900 mg, 2000 mg, or about 3000 mg. In some embodiments, the amount is between about 225 mg to about 1000 mg, e.g., about 675 mg or about 900 mg. An exemplary dosing regimen comprises administering an initial antibody dose of about 675 mg subcutaneously, followed by a monthly antibody dose of about 225 mg subcutaneously for, e.g., about two months, three months, four months, five months, six months, seven months, eight months, nine months, ten months, 11 months, or 12 months, or even a period of greater than one year (e.g., 18 months, two years, or three years). Another exemplary dosing regimen comprises administering an initial antibody dose of about 900 mg intravenously, followed by a monthly antibody dose of about 225 mg subcutaneously for, e.g., about two months, three months, four months, five months, six months, seven months, eight months, nine months, ten months, 11 months, or 12 months, or even a period of greater than one year (e.g., 18 months, two years, or three years). Yet another dosing regimen comprises administering an initial dose of about 900 mg intravenously in an infusion over about 60 minutes, followed by doses of about 900 mg administered intravenously in an infusion over about 60 minutes every quarter for one year, two years, three years, four years, or five years. However, other dosage regimens may be useful, depending on the pattern of pharmacokinetic decay that the practitioner wishes to achieve.

In some embodiments, the dose or amount of an antibody (e.g., monoclonal antibody that modulates the CGRP pathway, anti-CGRP antagonist antibody, monoclonal anti-CGRP antagonist antibody) described herein and administered to a subject may range from about 0.1 to 500, 0.1 to 100, 0.1 to 50, 0.1 to 20, 0.1 to 10, 1 to 10, 1 to 7, 1 to 5 or 0.1 to 3 mg/kg of body weight. In some embodiments, the dose or amount of an antibody (e.g., monoclonal antibody that modulates the CGRP pathway, anti-CGRP antagonist antibody, monoclonal anti-CGRP antagonist antibody) described herein and administered to a subject may be, may be at most, may be less than, or may be at least about 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 225, 250, 275, 300, 325, 350, 375, 400, 425, 450, 475, or about 500 mg/kg of body weight.

In some embodiments, the frequency at which a dose or amount of an antibody (e.g., monoclonal antibody that modulates the CGRP pathway, anti-CGRP antagonist antibody, monoclonal anti-CGRP antagonist antibody) described herein is administered to a subject may vary. In some embodiments, a single dose of antibody may be given to a subject across therapy. In some embodiments, the frequency at which a dose or amount of an antibody is administered to a subject is constant (e.g., administered about once per month or about once per quarter). In some embodiments, the frequency at which a dose or amount of an antibody is administered to a subject is about every quarter for about one year, two years, three years, four years, or five years. In some embodiments, the frequency at which a dose or amount of an antibody described herein is administered to a subject is variable (e.g., an initial dose followed by a dose at once per month, followed by additional doses at about three months and about seven months).

In some embodiments, the frequency at which an antibody is administered to a subject is, is at least, is less than, or is at most about one, two, three, four, five, or six time(s) per day. In some embodiments, the frequency at which an antibody (e.g., monoclonal antibody that modulates the CGRP pathway, anti-CGRP antagonist antibody, monoclonal anti-CGRP antagonist antibody) is administered to a subject is, is at least, is less than, or is at most about one, two, three, four, five, or six dose(s) per day.

In some embodiments, the frequency at which a dose or amount of an antibody (e.g., monoclonal antibody that modulates the CGRP pathway, anti-CGRP antagonist antibody, monoclonal anti-CGRP antagonist antibody) described herein is administered to a subject is, is at least, is less than, or is at most one, two, three, four, five, six, seven, eight, nine, ten, eleven, twelve, thirteen, fourteen, fifteen, sixteen, seventeen, eighteen, nineteen, or twenty time(s) per every one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, forty-one, forty-two, forty-three, forty-four, forty-five, forty-six, forty-seven, forty-eight, forty-nine, fifty, fifty-five, sixty, sixty-five, seventy, seventy-five, eighty, eighty-five, ninety, ninety-five, one-hundred, one-hundred twenty-five, one-hundred fifty, one-hundred eighty, or two-hundred day(s).

In some embodiments, the frequency at which a dose or amount of an antibody (e.g., monoclonal antibody that modulates the CGRP pathway, anti-CGRP antagonist antibody, monoclonal anti-CGRP antagonist antibody) described herein is administered to a subject is, is at least, is less than, or is at most one, two, three, four, five, six, seven, eight, nine, ten, eleven, twelve, thirteen, fourteen, fifteen, sixteen, seventeen, eighteen, nineteen, or twenty time(s) per every one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, forty-one, forty-two, forty-three, forty-four, forty-five, forty-six, forty-seven, forty-eight, forty-nine, fifty, fifty-five, sixty, sixty-five, seventy, seventy-five, eighty, eighty-five, ninety, ninety-five, or one-hundred week(s). In some embodiments, the frequency at which an antibody (e.g., monoclonal antibody that modulates the CGRP pathway, anti-CGRP antagonist antibody, monoclonal anti-CGRP antagonist antibody) described herein is administered to a subject is less than one, two, three, four, five, six, seven, eight, nine, ten, eleven, twelve, thirteen, fourteen, or fifteen dose(s) per week.

In some embodiments, the frequency at which a dose or amount of an antibody (e.g., monoclonal antibody that modulates the CGRP pathway, anti-CGRP antagonist antibody, monoclonal anti-CGRP antagonist antibody) is administered to a subject is, is at least, is less than, or is at most about one, two, three, four, five, six, seven, eight, nine, ten, eleven, twelve, thirteen, fourteen, fifteen, sixteen, seventeen, eighteen, nineteen, or twenty time(s) per every month, every two months, every three months, every four months, every five months, every six months, every seven months, every eight months, every nine months, every ten months, every eleven months, every twelve months, every thirteen months, every fourteen months, every fifteen months, every sixteen months, every seventeen months, or every eighteen month(s). In some embodiments, the frequency at which a dose or amount of an antibody (e.g., monoclonal antibody that modulates the CGRP pathway, anti-CGRP antagonist antibody, monoclonal anti-CGRP antagonist antibody) is administered to a subject is about one time per every one month. In some embodiments, the frequency at which a dose or amount of an antibody (e.g., monoclonal antibody that modulates the CGRP pathway, anti-CGRP antagonist antibody, monoclonal anti-CGRP antagonist antibody) is administered to a subject is about one time per every three months. In some embodiments, the frequency at which an antibody (e.g., monoclonal antibody that modulates the CGRP pathway, anti-CGRP antagonist antibody, monoclonal anti-CGRP antagonist antibody) described herein is administered to a subject is less than about one, two, three, four, five, six, seven, eight, nine, ten, eleven, twelve, thirteen, fourteen, or fifteen dose(s) per month. In some embodiments, a dose or amount of an antibody may be administered (e.g., subcutaneously or intravenously in an infusion) to a subject one time, two times, three times, four times, five times, six times, seven times, eight times, nine times, ten times or more per month.

In some embodiments, an antibody in a dose or amount of about 50 mg, 100 mg 150 mg, 200 mg, 225 mg, 250 mg, 300 mg, 350 mg, 400 mg, 450 mg, 500 mg, 550 mg, 600 mg, 650 mg, 675 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg, 2950 mg, 3000 mg, or more may be administered (e.g., subcutaneously or intravenously in an infusion) to a subject once per month. In some embodiments, an antibody in a dose or amount of between about 0.1 mg to 5000 mg, 1 mg to 4000 mg, 10 mg to 3000 mg, 10 mg to 2000 mg, 100 mg to 2000 mg, 150 mg to 2000 mg, 200 mg to 2000 mg, 250 mg to 2000 mg, 300 mg to 2000 mg, 350 mg to 2000 mg, 400 mg to 2000 mg, 450 mg to 2000 mg, 500 mg to 2000 mg, 550 mg to 2000 mg, 600 mg to 2000 mg, 650 mg to 2000 mg, 700 mg to 2000 mg, 750 mg to 2000 mg, 800 mg to 2000 mg, 850 mg to 2000 mg, 900 mg to 2000 mg, 950 mg to 2000 mg, or about 1000 mg to 2000 mg may be administered (e.g., subcutaneously or intravenously in an infusion) to a subject once per month. In some embodiments, between about 225 mg and about 1000 mg, e.g., about 225 mg of antibody are administered once per month. An exemplary dosing regimen comprises administering an initial antibody dose of about 675 mg subcutaneously, followed by a monthly antibody dose of about 225 mg subcutaneously for, e.g., about two months, three months, four months, five months, six months, seven months, eight months, nine months, ten months, 11 months, or 12 months, or even a period of greater than one year (e.g., 18 months, two years, or three years. An exemplary dosing regimen comprises administering an initial antibody dose of about 900 mg intravenously, followed by a monthly antibody dose of about 225 mg subcutaneously for, e.g., about two months, three months, four months, five months, six months, seven months, eight months, nine months, ten months, 11 months, or 12 months, or even a period of greater than one year (e.g., 18 months, two years, or three years. However, other dosage regimens may be useful, depending on the pattern of pharmacokinetic decay that the practitioner wishes to achieve.

In some embodiments, an antibody in a dose or amount of about 50 mg, 100 mg 150 mg, 200 mg, 225 mg, 250 mg, 300 mg, 350 mg, 400 mg, 450 mg, 500 mg, 550 mg, 600 mg, 650 mg, 675 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg, 2950 mg, 3000 mg, or more may be administered (e.g., subcutaneously or intravenously in an infusion) to a subject every three months. In some embodiments, an antibody in a dose or amount of between about 0.1 mg to 5000 mg, 1 mg to 4000 mg, 10 mg to 3000 mg, 10 mg to 2000 mg, 100 mg to 2000 mg, 150 mg to 2000 mg, 200 mg to 2000 mg, 250 mg to 2000 mg, 300 mg to 2000 mg, 350 mg to 2000 mg, 400 mg to 2000 mg, 450 mg to 2000 mg, 500 mg to 2000 mg, 550 mg to 2000 mg, 600 mg to 2000 mg, 650 mg to 2000 mg, 700 mg to 2000 mg, 750 mg to 2000 mg, 800 mg to 2000 mg, 850 mg to 2000 mg, 900 mg to 2000 mg, 950 mg to 2000 mg, or 1000 mg to 2000 mg may be administered (e.g., subcutaneously or intravenously in an infusion) to a subject every three months. In some embodiments, between about 225 mg to about 1000 mg is administered once every three months or less, e.g., about 900 mg is administered every three months intravenously in an infusion. However, other dosage regimens may be useful, depending on the pattern of pharmacokinetic decay that the practitioner wishes to achieve.

In some embodiments, an antibody in a dose or amount of about 50 mg, 100 mg 150 mg, 200 mg, 225 mg, 250 mg, 300 mg, 350 mg, 400 mg, 450 mg, 500 mg, 550 mg, 600 mg, 650 mg, 675 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg, 2950 mg, 3000 mg, or more may be administered (e.g., subcutaneously or intravenously in an infusion) to a subject every six months. In some embodiments, an antibody in a dose or amount of between about 0.1 mg to 5000 mg, 1 mg to 4000 mg, 10 mg to 3000 mg, 10 mg to 2000 mg, 100 mg to 2000 mg, 150 mg to 2000 mg, 200 mg to 2000 mg, 250 mg to 2000 mg, 300 mg to 2000 mg, 350 mg to 2000 mg, 400 mg to 2000 mg, 450 mg to 2000 mg, 500 mg to 2000 mg, 550 mg to 2000 mg, 600 mg to 2000 mg, 650 mg to 2000 mg, 700 mg to 2000 mg, 750 mg to 2000 mg, 800 mg to 2000 mg, 850 mg to 2000 mg, 900 mg to 2000 mg, 950 mg to 2000 mg, or 1000 mg to 2000 mg may be administered (e.g., subcutaneously or intravenously in an infusion) to a subject every six months. In some embodiments, between 225 mg to 1000 mg is administered once every six months or less. However, other dosage regimens may be useful, depending on the pattern of pharmacokinetic decay that the practitioner wishes to achieve.

In some embodiments, the frequency at which a dose or amount of an antibody (e.g., monoclonal antibody that modulates the CGRP pathway, anti-CGRP antagonist antibody, monoclonal anti-CGRP antagonist antibody) is administered to a subject (e.g., subcutaneously or intravenously) is, is at least, is less than, or is at most one, two, three, four, five, six, seven, eight, nine, ten, eleven, twelve, thirteen, fourteen, fifteen, sixteen, seventeen, eighteen, nineteen, or twenty time(s) per every quarter. As can be appreciated, a “quarter” can refer to a time period of a quarter year or may also refer to a calendar quarter such as a time period of January 1-March 31, April 1-June 30, July 1-September 30, or October 1-December 31. In some cases, a “quarter” may refer to a time period of approximately three months.

In some embodiments, an antibody in a dose or amount of about 50 mg, 100 mg 150 mg, 200 mg, 225 mg, 250 mg, 300 mg, 350 mg, 400 mg, 450 mg, 500 mg, 550 mg, 600 mg, 650 mg, 675 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg, 2950 mg, 3000 mg, or more may be administered (e.g., subcutaneously or intravenously in an infusion) to a subject every quarter. In some embodiments, an antibody in a dose or amount of between about 0.1 mg to 5000 mg, 1 mg to 4000 mg, 10 mg to 3000 mg, 10 mg to 2000 mg, 100 mg to 2000 mg, 150 mg to 2000 mg, 200 mg to 2000 mg, 250 mg to 2000 mg, 300 mg to 2000 mg, 350 mg to 2000 mg, 400 mg to 2000 mg, 450 mg to 2000 mg, 500 mg to 2000 mg, 550 mg to 2000 mg, 600 mg to 2000 mg, 650 mg to 2000 mg, 700 mg to 2000 mg, 750 mg to 2000 mg, 800 mg to 2000 mg, 850 mg to 2000 mg, 900 mg to 2000 mg, 950 mg to 2000 mg, or 1000 mg to 2000 mg may be administered (e.g., subcutaneously or intravenously in an infusion) to a subject every quarter. Yet another dosing regimen comprises administering an initial dose of about 900 mg intravenously in an infusion over about 60 minutes, followed by doses of about 900 mg administered intravenously in an infusion over about 60 minutes every quarter for one year, two years, three years, four years, or five years. However, other dosage regimens may be useful, depending on the pattern of pharmacokinetic decay that the practitioner wishes to achieve.

In some embodiments, the frequency at which a dose or amount of an antibody (e.g., monoclonal antibody that modulates the CGRP pathway, anti-CGRP antagonist antibody, monoclonal anti-CGRP antagonist antibody) is administered is, is at least, is less than, or is at most about one, two, three, four, five, six, seven, eight, nine, ten, eleven, twelve, thirteen, fourteen, fifteen, sixteen, seventeen, eighteen, nineteen, or twenty time(s) per every year, every two years, every three years, every four years, or every five years. In some embodiments, the frequency at which an antibody (e.g., monoclonal antibody that modulates the CGRP pathway, anti-CGRP antagonist antibody, monoclonal anti-CGRP antagonist antibody) is administered to a subject is less than one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four or twenty-five dose(s) per year.

In some embodiments, an antibody in a dose or amount of about 50 mg, 100 mg 150 mg, 200 mg, 225 mg, 250 mg, 300 mg, 350 mg, 400 mg, 450 mg, 500 mg, 550 mg, 600 mg, 650 mg, 675 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg, 2950 mg, 3000 mg, or more may be administered to a subject once per year. In some embodiments, an antibody in a dose or amount of between about 0.1 mg to 5000 mg, 1 mg to 4000 mg, 10 mg to 3000 mg, 10 mg to 2000 mg, 100 mg to 2000 mg, 150 mg to 2000 mg, 200 mg to 2000 mg, 250 mg to 2000 mg, 300 mg to 2000 mg, 350 mg to 2000 mg, 400 mg to 2000 mg, 450 mg to 2000 mg, 500 mg to 2000 mg, 550 mg to 2000 mg, 600 mg to 2000 mg, 650 mg to 2000 mg, 700 mg to 2000 mg, 750 mg to 2000 mg, 800 mg to 2000 mg, 850 mg to 2000 mg, 900 mg to 2000 mg, 950 mg to 2000 mg, or 1000 mg to 2000 mg may be administered to a subject every once per year. In some embodiments, between about 450 mg and about 2000 mg is administered once every year or less.

In some embodiments, a method may comprise administering an antibody (e.g., monoclonal antibody that modulates the CGRP pathway, anti-CGRP antagonist antibody, monoclonal anti-CGRP antagonist antibody) described herein to a subject on a plurality of days. Two, three, four, five, six, seven, eight or more days of the plurality of days may be more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75 or more days apart. In some embodiments, two of the plurality of days are more than one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty or more days apart. Moreover, in some embodiments, the amount of antibody administered on a first day of the plurality of days may be different (e.g., higher or lower) than the amount of the antibody administered on a second day.

In some embodiments, an initial dose (which can also be referred to as a loading dose or a starting dose) of an antibody (e.g., monoclonal antibody that modulates the CGRP pathway, anti-CGRP antagonist antibody, monoclonal anti-CGRP antagonist antibody) described herein may be administered to a subject, followed by administration of one or more additional doses at desired intervals. In some embodiments, the initial dose (or starting dose) and one or more of the additional doses are the same dose. In some embodiments, the one or more additional doses are a different dose than the initial or starting dose. In some embodiments, the initial dose and one or more of the additional doses are administered the same way, i.e., subcutaneously or intravenously. In some embodiments, the one or more additional doses are administered in a different way than the initial dose, e.g., the initial dose may be administered intravenously and the one or more additional doses may be administered subcutaneously. In some embodiments, the frequency at which the one or more additional doses are administered is constant (e.g., every month or every three months). In some embodiments, the frequency at which the one or more additional doses are administered is variable (e.g., one additional dose administered at one month following the initial dose, followed by another additional dose at three months following the initial dose). Any desirable and/or therapeutic regimen of initial loading dose, additional doses, and frequency (e.g., including those described herein) of additional doses may be used. An exemplary regimen includes an initial loading dose of about 675 mg anti-CGRP antagonist antibody administered subcutaneously, followed by subsequent maintenance doses of about 225 mg of the antibody administered subcutaneously at one month intervals. Another exemplary dosing regimen comprises an initial loading dose of about 900 mg anti-CGRP antagonist antibody administered intravenously, followed by subsequent maintenance doses of about 225 mg of the antibody administered subcutaneously at one month intervals. Yet another exemplary regimen includes an initial dose of about 900 mg anti-CGRP antagonist antibody administered intravenously in an infusion over about 60 minutes, followed by subsequent maintenance doses of about 900 mg anti-CGRP antagonist antibody administered intravenously in an infusion over about 60 minutes at three month intervals.

In some embodiments, an initial dose (or starting dose) of an antibody (e.g., monoclonal antibody that modulates the CGRP pathway, anti-CGRP antagonist antibody, monoclonal anti-CGRP antagonist antibody) of about 0.1 μg, 1 μg, 100 μg, 1 mg, 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1500 mg, 2000 mg, or about 3000 mg may be administered to a subject followed by one or more additional doses of the antibody of about 0.1 μg, 1 μg, 100 μg, 1 mg, 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1500 mg, 2000 mg, or about 3000 mg. An exemplary regimen includes an initial loading dose of about 675 mg anti-CGRP antagonist antibody administered subcutaneously, followed by subsequent maintenance doses of about 225 mg of the antibody administered subcutaneously at one month intervals. An exemplary regimen includes an initial loading dose of about 900 mg anti CGRP antagonist antibody administered intravenously, followed by subsequent maintenance doses of about 225 mg of the antibody administered subcutaneously at one month intervals. Yet another exemplary regimen includes an initial dose of about 900 mg anti-CGRP antagonist antibody administered intravenously in an infusion over about 60 minutes, followed by subsequent maintenance doses of about 900 mg anti-CGRP antagonist antibody administered intravenously in an infusion over about 60 minutes at three month intervals.

In some embodiments, a dose or amount of antibody (e.g., monoclonal antibody that modulates the CGRP pathway, anti-CGRP antagonist antibody, monoclonal anti-CGRP antagonist antibody) described herein may be divided into sub-doses and administered as multiple sub-doses, depending, for example, on the route of administration and/or particular formulation administered. For example, in cases where a dose is administered subcutaneously, the subcutaneous dose may be divided into multiple sub-doses and each sub-dose administered at a different site in order to avoid, for example, a larger, single subcutaneous injection at a single site. For example, an intravenous dose of 900 mg may be divided into four sub-doses of 225 mg each. As another example, a subcutaneous dose of 675 mg may be divided into three sub-doses of 225 mg each and each 225 mg dose may be administered at a different site, which can help minimize the volume injected at each site. The division of sub-doses may be equal (e.g., three equal sub-doses) or may be unequal (e.g., three sub-doses, two of the sub-doses twice as large as the other sub-doses).

In some embodiments, the number of doses of antibody administered to a subject over the course of treatment may vary depending upon, for example, achieving reduced incidence of a CCH or ECH and/or secondary symptom associated with a CCH or ECH in the subject. For example, the number of doses administered over the course of treatment may be, may be at least, or may be at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or treatment may be given indefinitely. In some cases, treatment may be acute such that at most 1, 2, 3, 4, 5, or 6 doses are administered to a subject for treatment.

In some embodiments, a dose (or sub-dose) or amount of an antibody (e.g., monoclonal antibody that modulates the CGRP pathway, anti-CGRP antagonist antibody, monoclonal anti-CGRP antagonist antibody) described herein may be formulated in a liquid formulation and administered (e.g., via subcutaneous injection, via intravenous injection) to a subject. In such cases, the volume of liquid formulation comprising antibody may vary depending upon, for example, the concentration of antibody in the liquid formulation, the desired dose of antibody, and/or the route of administration used. For example, the volume of liquid formulation comprising an antibody described herein and administered (e.g., via an injection, such as, for example, a subcutaneous injection or an intravenous infusion) to a subject may be from about 0.001 mL to about 10.0 mL, about 0.01 mL to about 5.0 mL, about 0.1 mL to about 5 mL, about 0.1 mL to about 3 mL, about 0.5 mL to about 2.5 mL, or about 1 mL to about 2.5 mL. For example, the volume of liquid formulation comprising an antibody (e.g., monoclonal antibody that modulates the CGRP pathway, anti-CGRP antagonist antibody, monoclonal anti-CGRP antagonist antibody) described herein and administered (e.g., via an injection, such as, for example, a subcutaneous injection, or an intravenous infusion) to a subject may be, may be at least, may be less than, or may be at most about 0.001, 0.005, 0.01, 0.02, 0.03, 0.04, 0.05, 0.06, 0.07, 0.08, 0.09, 0.10, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, 8.0, 8.5, 9.0, 9.5, or about 10.0 mL.

In some embodiments, a dose (or sub-dose) or amount of an antibody (e.g., monoclonal antibody that modulates the CGRP pathway, anti-CGRP antagonist antibody, monoclonal anti-CGRP antagonist antibody) described herein may be supplied in prefilled receptacles useful in administering antibody to a subject. Such prefilled receptacles may be designed for self-administration or for administration by another. For example, a dose (or sub-dose) or amount of antibody described herein may be supplied as a liquid formulation in pre-filled syringes, pre-filled syringes with a needle safety device, injection pens, or auto-injectors. In such examples, the pre-filled syringes may be designed for self-administration or for administration by another. In some cases, the pre-filled syringes or auto-injectors may be designed for subcutaneous administration and/or intravenous administration.

For the purpose of the present invention, the appropriate dosage of an antibody may depend on the antibody (or compositions thereof) employed, the type and severity of the secondary symptom, the type and severity of the CCH or ECH or other condition to be treated, whether the agent is administered for preventive or therapeutic purposes, previous therapy, the patient's clinical history and response to the agent, and the discretion of the attending physician. Typically, the clinician will administer an antibody, until a dosage is reached that achieves the desired result. Dose and/or frequency can vary over course of treatment.

Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of CCH or ECH or other condition. Alternatively, sustained continuous release formulations of antibodies may be appropriate. Various formulations and devices for achieving sustained release are known in the art.

In one embodiment, dosages for an antibody (e.g., monoclonal antibody that modulates the CGRP pathway, anti-CGRP antagonist antibody, monoclonal anti-CGRP antagonist antibody) described herein may be determined empirically in individuals who have been given one or more administration(s) of the antibody. Individuals are given incremental dosages of an antibody. To assess efficacy of an antibody, an indicator of the disease can be followed.

Administration of an antibody (e.g., monoclonal antibody that modulates the CGRP pathway, anti-CGRP antagonist antibody, monoclonal anti-CGRP antagonist antibody) in accordance with the methods of the present invention can be continuous or intermittent, depending, for example, upon the recipient's physiological condition, whether the purpose of the administration is therapeutic or prophylactic, and other factors known to skilled practitioners. The administration of an antibody may be essentially continuous over a preselected period of time or may be in a series of spaced dose, e.g., either before, during, or after developing CCH or ECH; before; during; before and after; during and after; before and during; or before, during, and after developing CCH or ECH. Administration can be before, during and/or after any event likely to give rise to CCH or ECH.

In some embodiments, more than one antibody may be present. At least one, at least two, at least three, at least four, at least five different, or more antibodies can be present. Generally, those antibodies may have complementary activities that do not adversely affect each other. An antibody (e.g., monoclonal antibody that modulates the CGRP pathway, anti-CGRP antagonist antibody, monoclonal anti-CGRP antagonist antibody) described herein can also be used in conjunction with other CGRP antagonists or CGRP receptor antagonists. For example, one or more of the following CGRP antagonists may be used: an anti-sense molecule directed to a CGRP (including an anti-sense molecule directed to a nucleic acid encoding CGRP), a CGRP inhibitory compound, a CGRP structural analog, a dominant-negative mutation of a CGRP receptor that binds a CGRP, and an anti-CGRP receptor antibody. An antibody can also be used in conjunction with other agents that serve to enhance and/or complement the effectiveness of the agents.

Diagnosis or assessment of CCH or ECH is well-established in the art. Assessment may be performed based on subjective measures, such as patient characterization of symptoms. In some embodiments, assessment of CCH or ECH may be via headache hours, as described elsewhere herein. For example, assessment of CCH or ECH may be in terms of daily headache hours, weekly headache hours, monthly headache hours and/or yearly headache hours. In some cases, headache hours may be as reported by the subject.

Treatment efficacy can be assessed by methods well-known in the art. For example, pain relief may be assessed. Accordingly, in some embodiments, pain relief is subjectively observed after 1, 2, or a few hours after administering an anti-CGRP antibody. In some embodiments, frequency of CCH or ECH attacks is subjectively observed after administering an anti-CGRP antibody.

In some embodiments, a method for preventing, treating, or reducing incidence of CCH or ECH in a subject as described herein may reduce incidence of CCH or ECH after a single administration of an antibody (e.g., monoclonal antibody that modulates the CGRP pathway, anti-CGRP antagonist antibody, monoclonal anti-CGRP antagonist antibody) described herein for an extended period of time. For example, incidence of CCH or ECH may be reduced for at least 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more days after a single administration.

In some embodiments, a method for treating or reducing incidence of CCH or ECH in a subject as described herein may reduce the number of headache hours experienced by a subject from a pre-administration level after administration of one or more doses of an antibody (e.g., monoclonal antibody that modulates the CGRP pathway, anti-CGRP antagonist antibody, monoclonal anti-CGRP antagonist antibody) described herein to the subject. For example, daily headache hours experienced by the subject after administering one or more doses of an antibody to the subject may be reduced by 0.5, 1, 2, 3, 4, 5, 6, 7, 8, 9, 10, 11, 12, 13, 14, 15, 16, 17, 18, 19, 20, 21, 22, 23, or 24 headache hours from a pre-administration level in the subject. In some cases, daily headache hours experienced by the subject after administering one or more doses of an antibody to the subject may be reduced by 0.5%, 1%, 5%, 10%, 15%, 20%, 25%, 30%, 35%, 40%, 45%, 50%, 55%, 60%, 65%, 70%, 75%, 80%, 85%, 90%, 95%, 99%, or more relative to a pre-administration level in the subject. In another example, weekly headache hours experienced by the subject after administering one or more doses of an antibody to the subject may be reduced by 0.5, 1, 5, 10, 15, 20, 25, 30, 35, 40, 45, 50, 55, 60, 65, 70, 75 or more headache hours from a pre-administration level in the subject. In some cases, weekly headache hours experienced by the subject after administering one or more doses of an antibody to the subject may be reduced by 0.5%, 1%, 5%, 10%, 15%, 20%, 25%, 30%, 35%, 40%, 45%, 50%, 55%, 60%, 65%, 70%, 75%, 80%, 85%, 90%, 95%, 99%, or more relative to a pre-administration level in the subject. In another example, monthly headache hours experienced by the subject after administering one or more doses of an antibody to the subject may be reduced by 0.5, 1, 5, 10, 15, 20, 25, 30, 35, 40, 45, 50, 55, 60, 65, 70, 75, 80, 85, 90, 95, 100, 105, 110, 115, 120, 125, or more headache hours from a pre-administration level. In some cases, monthly headache hours experienced by the subject after administering one or more doses of an antibody to the subject may be reduced by 0.5%, 1%, 5%, 10%, 15%, 20%, 25%, 30%, 35%, 40%, 45%, 50%, 55%, 60%, 65%, 70%, 75%, 80%, 85%, 90%, 95%, 99% or more relative to a pre-administration level in the subject.

In some embodiments, a method for treating or reducing incidence of CCH or ECH in a subject as described herein may reduce the number of headache days experienced by a subject from a pre-administration level after administration of one or more doses of an antibody (e.g., monoclonal antibody that modulates the CGRP pathway, anti-CGRP antagonist antibody, monoclonal anti-CGRP antagonist antibody) described herein to the subject. For example, weekly headache days experienced by the subject after administering one or more doses of an antibody to the subject may be reduced by 0.5, 1, 1.5, 2, 2.5, 3, 3.5, 4, 4.5, 5, 5.5, 6, 6.5, or 7 headache days from a pre-administration level in the subject. In some cases, weekly headache days experienced by the subject after administering one or more doses of an antibody to the subject may be reduced by 0.5%, 1%, 5%, 10%, 15%, 20%, 25%, 30%, 35%, 40%, 45%, 50%, 55%, 60%, 65%, 70%, 75%, 80%, 85%, 90%, 95%, 99% or more relative to a pre-administration level in the subject. In another example, monthly headache days experienced by the subject after administering one or more doses of an antibody to the subject may be reduced by 0.5, 1, 2, 3, 4, 5, 6, 7, 8, 9, 10, 15, 20 or more headache days from a pre-administration level.

In some embodiments, a method may comprise administering to a subject one or more additional agent(s) simultaneously or sequentially with an antibody (e.g., monoclonal antibody that modulates the CGRP pathway, anti-CGRP antagonist antibody, monoclonal anti-CGRP antagonist antibody). In some embodiments, an additional agent may be an anti-headache medication such as an example anti-headache medication (e.g., 5-HT1 agonists, triptans, ergot alkaloids, opiates, β-adrenergic antagonists, NSAIDs) described elsewhere herein. In some embodiments, a therapeutic effect may be greater as compared to use of an antibody or one or more additional agent(s) alone. Accordingly, a synergistic effect between an antibody and the one or more additional agents may be achieved. In some embodiments, the one or more additional agent(s) may be taken by a subject prophylactically.

### B. Anti-CGRP Antagonist Antibodies

In some embodiments, the methods of the invention use an antibody, which can be an anti-CGRP antagonist antibody. An anti-CGRP antagonist antibody can refer to any antibody molecule that blocks, suppresses or reduces (including significantly) CGRP biological activity, including downstream pathways mediated by CGRP signaling, such as receptor binding and/or elicitation of a cellular response to CGRP.

An anti-CGRP antagonist antibody can exhibit any one or more of the following characteristics: (a) bind to CGRP; (b) block CGRP from binding to its receptor(s); (c) block or decrease CGRP receptor activation (including, but not limited to, cAMP activation); (d) inhibit CGRP biological activity or downstream pathways mediated by CGRP signaling function; (e) prevent, ameliorate, or treat any aspect of CCH or ECH; (f) increase clearance of CGRP; and (g) inhibit (reduce) CGRP synthesis, production or release. Anti-CGRP antagonist antibodies are known in the art. See e.g., Tan et al., Clin. Sci. (Lond). 89:565-73, 1995; Sigma (Missouri, US), product number C7113 (clone #4901); Plourde et al., Peptides 14:1225-1229, 1993.

In some embodiments, the antibody reacts with CGRP in a manner that inhibits CGRP, and/or the CGRP pathway, including downstream pathways mediated by the CGRP signaling function. In some embodiments, the anti-CGRP antagonist antibody recognizes human CGRP. In some embodiments, the anti-CGRP antagonist antibody binds to both human α-CGRP and β-CGRP. In some embodiments, the anti-CGRP antagonist antibody binds human and rat CGRP. In some embodiments, the anti-CGRP antagonist antibody binds the C-terminal fragment having amino acids 25-37 of CGRP. In some embodiments, the anti-CGRP antagonist antibody binds a C-terminal epitope within amino acids 25-37 of CGRP.

The antibodies useful in the present invention can encompass monoclonal antibodies, polyclonal antibodies, antibody fragments (e.g., Fab, Fab′, F(ab′)2, Fv, Fc, etc.), chimeric antibodies, bispecific antibodies, heteroconjugate antibodies, single chain (ScFv), mutants thereof, fusion proteins comprising an antibody portion (e.g., a domain antibody), humanized antibodies, and any other modified configuration of the immunoglobulin molecule that comprises an antigen recognition site of the required specificity, including glycosylation variants of antibodies, amino acid sequence variants of antibodies, and covalently modified antibodies. The antibodies may be murine, rat, human, or any other origin (including chimeric or humanized antibodies).

In some embodiments, the anti-CGRP antagonist antibody is a monoclonal antibody. In some embodiments, the anti-CGRP antagonist antibody is humanized. In some embodiments, the antibody is human. In some embodiments, the anti-CGRP antagonist antibody is antibody G1 (as described herein). In some embodiments, the anti-CGRP antagonist antibody comprises one or more CDR(s) (such as one, two, three, four, five, or, in some embodiments, all six CDRs) of antibody G1 or variants of G1 shown in Table 6. In still other embodiments, the anti-CGRP antagonist antibody comprises the amino acid sequence of the heavy chain variable region shown in FIG. 5 (SEQ ID NO:1) and the amino acid sequence of the light chain variable region shown in FIG. 5 (SEQ ID NO:2). In still other embodiments, the anti-CGRP antagonist antibody comprises a heavy chain full antibody amino acid sequence shown in SEQ ID NO:11 and a light chain full antibody amino acid sequence shown in SEQ ID NO:12.

In some embodiments, the antibody comprises a light chain variable region (LCVR) and a heavy chain variable region (HCVR) selected from the groups consisting of: (a) LCVR17 (SEQ ID NO:58) and HCVR22 (SEQ ID NO:59); (b) LCVR18 (SEQ ID NO:60) and HCVR23 (SEQ ID NO:61); (c) LCVR19 (SEQ ID NO:62) and HCVR24 (SEQ ID NO:63); (d) LCVR20 (SEQ ID NO:64) and HCVR25 (SEQ ID NO:65); (e) LCVR21 (SEQ ID NO:66) and HCVR26 (SEQ ID NO:67); (f) LCVR27 (SEQ ID NO:68) and HCVR28 (SEQ ID NO:69); (g) LCVR29 (SEQ ID NO:70) and HCVR30 (SEQ ID NO:71); (h) LCVR31 (SEQ ID NO:72) and HCVR32 (SEQ ID NO:73); (i) LCVR33 (SEQ ID NO:74) and HCVR34 (SEQ ID NO:75); (j) LCVR35 (SEQ ID NO:76) and HCVR36 (SEQ ID NO:77); and (k) LCVR37 (SEQ ID NO:78) and HCVR38 (SEQ ID NO:79). Sequences of these regions are provided herein. Other examples of anti-CGRP antibodies are described in US20110305711 (SEQ ID NOs:5, 6, 7, 12, 16, 19, 24, 29, 34, and 39), US20120294802, US20120294797 (SEQ ID NOs:51-60), which are hereby incorporated by reference in their entireties. For example, antibodies with any of the following sequences may be used.

Ab6 Variable region Light chain (humanized)
(SEQ ID NO: 80)
protein sequence (US20120294797)
QVLTQSPSSLSASVGDRVTINCQASQSVYHNTYLAWYQQKPGKVPKQLIY
DASTLASGVPSRFSGSGSGTDFTLTISSLQPEDVATYYCLGSYDCTNGDC
FVFGGGTKVEIKR
Ab6 Light chain (humanized) Full length protein
sequence (US20120294797)
(SEQ ID NO: 81)
QVLTQSPSSLSASVGDRVTINCQASQSVYHNTYLAWYQQKPGKVPKQLIY
DASTLASGVPSRFSGSGSGTDFTLTISSLQPEDVATYYCLGSYDCTNGDC
FVFGGGTKVEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
Ab6 Variable region heavy chain (humanized)
protein sequence (US20120294797)
(SEQ ID NO: 82)
EVQLVESGGGLVQPGGSLRLSCAVSGIDLSGYYMNWVRQAPGKGLEWVGV
IGINGATYYASWAKGRFTISRDNSKTTVYLQMNSLRAEDTAVYFCARGDI
WGQGTLVTVSS
Ab6 Heavy chain (humanized) Full length protein
sequence - yeast produced (US20120294797)
(SEQ ID NO: 83)
EVQLVESGGGLVQPGGSLRLSCAVSGIDLSGYYMNWVRQAPGKGLEWVGV
IGINGATYYASWAKGRFTISRDNSKTTVYLQMNSLRAEDTAVYFCARGDI
WGQGTLVTVSSASTKGPSVFPLAPSSKSTSGGTAALGCLVKDYFPEPVTV
SWNSGALTSGVHTFPAVLQSSGLYSLSSVVTVPSSSLGTQTYICNVNHKP
SNTKVDARVEPKSCDKTHTCPPCPAPELLGGPSVFLFPPKPKDTLMISRT
PEVTCVVVDVSHEDPEVKFNWYVDGVEVHNAKTKPREEQYASTYRVVSVL
TVLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFFL
YSKLTVDKSRWQQGNVFSCSVMHEALHNHYTQKSLSLSPGK
Ab6 Variable region Light chain (humanized)
protein sequence CDRI (US20120294797)
(SEQ ID NO: 84)
QASQSVYHNTYLA
Ab6 Variable region Light chain (humanized)
protein sequence CDR2 (US20120294797)
(SEQ ID NO: 85)
DASTLAS
Ab6 Variable region Light chain (humanized)
protein sequence CDR3 (US20120294797)
(SEQ ID NO: 86)
LGSYDCTNGDCFV
Ab6 Variable region heavy chain (humanized)
protein sequence CDRI (US20120294797)
(SEQ ID NO: 87)
GYYMN
Ab6 Variable region heavy chain (humanized)
protein sequence CDR2 (US20120294797)
(SEQ ID NO: 88)
IGINGATYYASWAKG
Ab6 Variable region heavy chain (humanized)
protein sequence CDR3 (US20120294797)
(SEQ ID NO: 89)
GDI
Light chain variable region protein sequence CDR3
(US20110305711)
(SEQ ID NO: 90)
QQGDALPPT
Light chain variable region protein sequence CDR1
(US20110305711)
(SEQ ID NO: 91)
RASKDISKYL
Light chain variable region protein sequence CDR2
(US20110305711)
(SEQ ID NO: 92)
YTSGYSH
Heavy chain variable region protein sequence CDR1
(US20110305711)
(SEQ ID NO: 93)
GYTFGNYWMQ
Heavy chain variable region protein sequence CDR2
(US20110305711)
(SEQ ID NO: 94)
AIYEGTGKTVYIQKFAD
Heavy chain variable region protein sequence CDR3
(US20110305711)
(SEQ ID NO: 95)
LSDYVSGFGY
Light chain variable region protein sequence
(US20110305711)
(SEQ ID NO: 96)
DIQMTQSPSSLSASVGDRVTITCRASKDISKYLNWYQQKPGKAPKLLIYY
TSGYHSGVPSRFSGSGSGTDFTLTISSLQPEDFATYYCQQGDALPPTFGG
GTKVEIK
Heavy chain variable region protein sequence
(US20110305711)
(SEQ ID NO: 97)
QVQLVQSGAEVKKPGSSVKVSCKASGYTFGNYWMQWVRQAPGQGLEWMGA
IYEGTGKTVYIQKFADRVTITADKSTSTAYMELSSLRSEDTAVYYCARLS
DYVSGFGYWGQGTTVTVSS
Light chain protein sequence (US20110305711)
(SEQ ID NO: 98)
DIQMTQSPSSLSASVGDRVTITCRASKDISKYLNWYQQKPGKAPKLLIYY
TSGYHSGVPSRFSGSGSGTDFTLTISSLQPEDFATYYCQQGDALPPTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
Heavy chain protein sequence (US20110305711)
(SEQ ID NO: 99)
QVQLVQSGAEVKKPGSSVKVSCKASGYTFGNYWMQWVRQAPGQGLEWMGA
IYEGTGKTVYIQKFADRVTITADKSTSTAYMELSSLRSEDTAVYYCARLS
DYVSGFGYWGQGTTVTVSSASTKGPSVFPLAPCSRSTSESTAALGCLVKD
YFPEPVTVSWNSGALTSGVHTFPAVLQSSGLYSLSSVVTVPSSSLGTKTY
TCNVDHKPSNTKVDKRVESKYGPPCPPCPAPEAAGGPSVFLFPPKPKDTL
MISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLG

In some embodiments, the antibody comprises a modified constant region, such as a constant region that is immunologically inert described herein. In some embodiments, the constant region is modified as described in Eur. J. Immunol. (1999) 29:2613-2624; PCT Application No. PCT/GB99/01441; and/or UK Patent Application No. 9809951.8. In other embodiments, the antibody comprises a human heavy chain IgG2 constant region comprising the following mutations: A330P331 to S330S331 (amino acid numbering with reference to the wildtype IgG2 sequence). Eur. J. Immunol. (1999) 29:2613-2624. In some embodiments, the antibody comprises a constant region of IgG4 comprising the following mutations: E233F234L235 to P233V234A235. In still other embodiments, the constant region is aglycosylated for N-linked glycosylation. In some embodiments, the constant region is aglycosylated for N-linked glycosylation by mutating the oligosaccharide attachment residue (such as Asn297) and/or flanking residues that are part of the N-glycosylation recognition sequence in the constant region. In some embodiments, the constant region is aglycosylated for N-linked glycosylation. The constant region may be aglycosylated for N-linked glycosylation enzymatically or by expression in a glycosylation deficient host cell.

The binding affinity (K_{D}) of an anti-CGRP antagonist antibody to CGRP (such as human α-CGRP) can be about 0.02 to about 200 nM. In some embodiments, the binding affinity is any of about 200 nM, about 100 nM, about 50 nM, about 10 nM, about 1 nM, about 500 pM, about 100 pM, about 60 pM, about 50 pM, about 20 pM, about 15 pM, about 10 pM, about 5 pM, or about 2 pM. In some embodiments, the binding affinity is less than any of about 250 nM, about 200 nM, about 100 nM, about 50 nM, about 10 nM, about 1 nM, about 500 pM, about 100 pM, or about 50 pM.

One way of determining binding affinity of antibodies to CGRP is by measuring binding affinity of monofunctional Fab fragments of the antibody. To obtain monofunctional Fab fragments, an antibody (for example, IgG) can be cleaved with papain or expressed recombinantly. The affinity of an anti-CGRP Fab fragment of an antibody can be determined by surface plasmon resonance (Biacore3000™ surface plasmon resonance (SPR) system, Biacore, INC, Piscataway N.J.) equipped with pre-immobilized streptavidin sensor chips (SA) using HBS-EP running buffer (0.01M HEPES, pH 7.4, 0.15 NaCl, 3 mM EDTA, 0.005% v/v Surfactant P20). Biotinylated human CGRP (or any other CGRP) can be diluted into HBS-EP buffer to a concentration of less than 0.5 μg/mL and injected across the individual chip channels using variable contact times, to achieve two ranges of antigen density, either 50-200 response units (RU) for detailed kinetic studies or 800-1,000 RU for screening assays. Regeneration studies have shown that 25 mM NaOH in 25% v/v ethanol effectively removes the bound Fab while keeping the activity of CGRP on the chip for over 200 injections. Typically, serial dilutions (spanning concentrations of 0.1-10× estimated K_{D}) of purified Fab samples are injected for 1 min at 100 μL/minute and dissociation times of up to 2 hours are allowed. The concentrations of the Fab proteins are determined by ELISA and/or SDS-PAGE electrophoresis using a Fab of known concentration (as determined by amino acid analysis) as a standard. Kinetic association rates (kₒₙ) and dissociation rates (k_{off}) are obtained simultaneously by fitting the data globally to a 1:1 Langmuir binding model (Karlsson, R. Roos, H. Fagerstam, L. Petersson, B. (1994). Methods Enzymology 6. 99-110) using the BIAevaluation program. Equilibrium dissociation constant (K_{D}) values are calculated as k_{off}/kₒₙ. This protocol is suitable for use in determining binding affinity of an antibody to any CGRP, including human CGRP, CGRP of another mammalian (such as mouse CGRP, rat CGRP, primate CGRP), as well as different forms of CGRP (such as a and β form). Binding affinity of an antibody is generally measured at 25° C., but can also be measured at 37° C.

Antibodies, including anti-CGRP antagonist antibodies, may be made by any method known in the art. The route and schedule of immunization of the host animal are generally in keeping with established and conventional techniques for antibody stimulation and production, as further described herein. General techniques for production of human and mouse antibodies are known in the art and are described herein.

It is contemplated that any mammalian subject including humans or antibody producing cells therefrom can be manipulated to serve as the basis for production of mammalian, including human, hybridoma cell lines. Typically, the host animal is inoculated intraperitoneally, intramuscularly, orally, subcutaneously, intraplantar, and/or intradermally with an amount of immunogen, including as described herein.

Antibodies (e.g., anti-CGRP antagonist antibodies) and polypeptides derived from antibodies can be identified or characterized using methods known in the art, whereby reduction, amelioration, or neutralization of a CGRP biological activity is detected and/or measured. For example, anti-CGRP antagonist antibody can also be identified by incubating a candidate agent with CGRP and monitoring any one or more of the following characteristics: (a) bind to CGRP; (b) block CGRP from binding to its receptor(s); (c) block or decrease CGRP receptor activation (including cAMP activation); (d) inhibit CGRP biological activity or downstream pathways mediated by CGRP signaling function; (e) prevent, ameliorate, or treat any aspect of CCH or ECH; (f) increase clearance of CGRP; and (g) inhibit (reduce) CGRP synthesis, production or release. In some embodiments, an anti-CGRP antagonist antibody or polypeptide is identified by incubating a candidate agent with CGRP and monitoring binding and/or attendant reduction or neutralization of a biological activity of CGRP. The binding assay may be performed with purified CGRP polypeptide(s), or with cells naturally expressing, or transfected to express, CGRP polypeptide(s). In one embodiment, the binding assay is a competitive binding assay, where the ability of a candidate antibody to compete with a known anti-CGRP antagonist for CGRP binding is evaluated. The assay may be performed in various formats, including the ELISA format. In other embodiments, an anti-CGRP antagonist antibody is identified by incubating a candidate agent with CGRP and monitoring binding and attendant inhibition of CGRP receptor activation expressed on the surface of a cell. In some embodiments, an anti-CGRP receptor antibody can be used in any of the methods described herein. For example, anti-CGRP receptor antibodies, as described in US20100172895 and U.S. Pat. No. 9,102,731, which are hereby incorporated by reference in their entireties, may be used. Therefore, antibodies with any of the following sequences may be used.

Light chain variable region protein sequence CDR1
(U.S. Pat. No. 9,102,731)
(SEQ ID NO: 100)
SGSSSNIGNNYVS
Light chain variable region protein sequence CDR2
(U.S. Pat. No. 9,102,731)
(SEQ ID NO: 101)
DNNKRPS
Light chain variable region protein sequence CDR3
(U.S. Pat. No. 9,102,731)
(SEQ ID NO: 102)
GTWDSRLSAVV
Heavy chain variable region protein sequence CDR1
(U.S. Pat. No. 9,102,731)
(SEQ ID NO: 103)
SFGMH
Heavy chain variable region protein sequence CDR2
(U.S. Pat. No. 9,102,731)
(SEQ ID NO: 104)
VISFDGSIKYSVDSVKG
Heavy chain variable region protein sequence CDR3
(U.S. Pat. No. 9,102,731)
(SEQ ID NO: 105)
DRLNYYDSSGYYHYKYYGMAV
Light chain variable region protein sequence
(U.S. Pat. No. 9,102,731)
(SEQ ID NO: 106)
QSVLTQPPSVSAAPGQKVTISCSGSSSNIGNNYVSWYQQLPGTAPKLLIY
DNNKRPSGIPDRFSGSKSGTSTTLGITGLQTGDEADYYCGTWDSRLSAVV
FGGGTKLTVL
Heavy chain variable region protein sequence
(U.S. Pat. No. 9,102,731)
(SEQ ID NO: 107)
QVQLVESGGGVVQPGRSLRLSCAASGFTFSSFGMHWVRQAPGKGLEWVAV
ISFDGSIKYSVDSVKGRFTISRDNSKNTLFLQMNSLRAEDTAVYYCARDR
LNYYDSSGYYHYKYYGMAVWGQGTTVTVSS
Light chain protein sequence
(U.S. Pat. No. 9,102,731)
(SEQ ID NO: 108)
MDMRVPAQLLGLLLLWLRGARCQSVLTQPPSVSAAPGQKVTISCSGSSSN
IGNNYVSWYQQLPGTAPKLLIYDNNKRPSGIPDRFSGSKSGTSTTLGITG
LQTGDEADYYCGTWDSRLSAVVFGGGTKLTVLGQPKANPTVTLFPPSSEE
LQANKATLVCLISDFYPGAVTVAWKADGSPVKAGVETTKPSKQSNNKYAA
SSYLSLTPEQWKSHRSYSCQVTHEGSTVEKTVAPTECS
Heavy chain protein sequence
(U.S. Pat. No. 9,102,731)
(SEQ ID NO: 109)
MDMRVPAQLLGLLLLWLRGARCQVQLVESGGGVVQPGRSLRLSCAASGFT
FSSFGMHWVRQAPGKGLEWVAVISFDGSIKYSVDSVKGRFTISRDNSKNT
LFLQMNSLRAEDTAVYYCARDRLNYYDSSGYYHYKYYGMAVWGQGTTVTV
SSASTKGPSVFPLAPCSRSTSESTAALGCLVKDYFPEPVTVSWNSGALTS
GVHTFPAVLQSSGLYSLSSVVTVPSSNFGTQTYTCNVDHKPSNTKVDKTV
ERKCCVECPPCPAPPVAGPSVFLFPPKPKDTLMISRTPEVTCVVVDVSHE
DPEVQFNWYVDGVEVHNAKTKPREEQFNSTFRVVSVLTVVHQDWLNGKEY
KCKVSNKGLPAPIEKTISKTKGQPREPQVYTLPPSREEMTKNQVSLTCLV
KGFYPSDIAVEWESNGQPENNYKTTPPMLDSDGSFFLYSKLTVDKSRWQQ
GNVFSCSVMHEALHNHYTQKSLSLSPGK

Following initial identification, the activity of a candidate antibody (e.g., anti-CGRP antagonist antibody) can be further confirmed and refined by bioassays, known to test the targeted biological activities. Animal models of CCH or ECH may further be used for testing efficacy of antagonist antibodies or polypeptides. Reuter, et al., Functional Neurology (15) Suppl. 3, 2000.

Antibodies, including anti-CGRP antagonist antibodies, may be characterized using methods well known in the art. For example, one method is to identify the epitope to which it binds, or “epitope mapping.” There are many methods known in the art for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999.

Yet another method which can be used to characterize an antibody, including an anti-CGRP antagonist antibody, is to use competition assays with other antibodies known to bind to the same antigen, i.e., various fragments on CGRP, to determine if the anti-CGRP antagonist antibody binds to the same epitope as other antibodies. Competition assays are well known to those of skill in the art.

### C. Antibody G1 and Related Antibodies, Polypeptides, Polynucleotides, Vectors and Host Cells

This invention encompasses compositions, including pharmaceutical compositions, comprising antibody G1 and its variants shown in Table 6 or polypeptide derived from antibody G1 and its variants shown in Table 6; and polynucleotides comprising sequences encoding G1 and its variants or the polypeptide. In some embodiments, compositions comprise one or more antibodies or polypeptides (which may or may not be an antibody) that bind to CGRP, and/or one or more polynucleotides comprising sequences encoding one or more antibodies or polypeptides that bind to CGRP. These compositions may further comprise suitable excipients, such as pharmaceutically acceptable excipients including buffers, which are well known in the art.

In some embodiments, the anti-CGRP antagonist antibodies and polypeptides of the invention are characterized by any (one or more) of the following characteristics: (a) bind to CGRP; (b) block CGRP from binding to its receptor(s); (c) block or decrease CGRP receptor activation (including cAMP activation); (d) inhibit CGRP biological activity or downstream pathways mediated by CGRP signaling function; (e) prevent, ameliorate, or treat any aspect of CCH or ECH; (f) increase clearance of CGRP; and (g) inhibit (reduce) CGRP synthesis, production or release.

In some embodiments, the invention provides any of the following, or compositions (including pharmaceutical compositions) comprising any of the following: (a) antibody G1 or its variants shown in Table 6; (b) a fragment or a region of antibody G1 or its variants shown in Table 6; (c) a light chain of antibody G1 or its variants shown in Table 6; (d) a heavy chain of antibody G1 or its variants shown in Table 6; (e) one or more variable region(s) from a light chain and/or a heavy chain of antibody G1 or its variants shown in Table 6; (f) one or more CDR(s) (one, two, three, four, five or six CDRs) of antibody G1 or its variants shown in Table 6; (g) CDR H3 from the heavy chain of antibody G1; (h) CDR L3 from the light chain of antibody G1 or its variants shown in Table 6; (i) three CDRs from the light chain of antibody G1 or its variants shown in Table 6; (j) three CDRs from the heavy chain of antibody G1 or its variants shown in Table 6; (k) three CDRs from the light chain and three CDRs from the heavy chain, of antibody G1 or its variants shown in Table 6; and (l) an antibody comprising any one of (b) through (k). In some embodiments, the invention also provides polypeptides comprising any one or more of the above.

The CDR portions of antibody G1 (including Chothia and Kabat CDRs) are diagrammatically depicted in FIG. 5. Determination of CDR regions is well within the skill of the art. It is understood that in some embodiments, CDRs can be a combination of the Kabat and Chothia CDR (also termed “combined CDRs” or “extended CDRs”). In some embodiments, the CDRs are the Kabat CDRs. In other embodiments, the CDRs are the Chothia CDRs. In other words, in embodiments with more than one CDR, the CDRs may be any of Kabat, Chothia, combination CDRs, or combinations thereof.

In some embodiments, the invention provides a polypeptide (which may or may not be an antibody) which comprises at least one CDR, at least two, at least three, or at least four, at least five, or all six CDRs that are substantially identical to at least one CDR, at least two, at least three, at least four, at least five or all six CDRs of G1 or its variants shown in Table 6. Other embodiments include antibodies which have at least two, three, four, five, or six CDR(s) that are substantially identical to at least two, three, four, five or six CDRs of G1 or derived from G1. In some embodiments, the at least one, two, three, four, five, or six CDR(s) are at least about 85%, 86%, 87%, 88%, 89%, 90%, 95%, 96%, 97%, 98%, or 99% identical to at least one, two, three, four, five or six CDRs of G1 or its variants shown in Table 6. It is understood that, for purposes of this invention, binding specificity and/or overall activity is generally retained, although the extent of activity may vary compared to G1 or its variants shown in Table 6 (may be greater or lesser).

In some embodiments, the invention also provides a polypeptide (which may or may not be an antibody) which comprises an amino acid sequence of G1 or its variants shown in Table 6 that has any of the following: at least 5 contiguous amino acids, at least 8 contiguous amino acids, at least about 10 contiguous amino acids, at least about 15 contiguous amino acids, at least about 20 contiguous amino acids, at least about 25 contiguous amino acids, at least about 30 contiguous amino acids of a sequence of G1 or its variants shown in Table 6, wherein at least 3 of the amino acids are from a variable region of G1 (FIG. 5) or its variants shown in Table 6. In one embodiment, the variable region is from a light chain of G1. In another embodiment, the variable region is from a heavy chain of G1. An exemplary polypeptide has contiguous amino acid (lengths described above) from both the heavy and light chain variable regions of G1. In another embodiment, the 5 (or more) contiguous amino acids are from a complementarity determining region (CDR) of G1 shown in FIG. 5. In some embodiments, the contiguous amino acids are from a variable region of G1.

The binding affinity (K_{D}) of an anti-CGRP antagonist antibody and polypeptide to CGRP (such as human α-CGRP) can be about 0.06 to about 200 nM. In some embodiments, the binding affinity is any of about 200 nM, 100 nM, about 50 nM, about 10 nM, about 1 nM, about 500 pM, about 100 pM, about 60 pM, about 50 pM, about 20 pM, about 15 pM, about 10 pM, about 5 pM, or about 2 pM. In some embodiments, the binding affinity is less than any of about 250 nM, about 200 nM, about 100 nM, about 50 nM, about 10 nM, about 1 nM, about 500 pM, about 100 pM, or about 50 pM.

The antibodies provided herein can be made by procedures known in the art. The polypeptides can be produced by proteolytic or other degradation of the antibodies, by recombinant methods (i.e., single or fusion polypeptides) as described above or by chemical synthesis. Polypeptides of the antibodies, especially shorter polypeptides up to about 50 amino acids, are conveniently made by chemical synthesis. Methods of chemical synthesis are known in the art and are commercially available. For example, an antibody could be produced by an automated polypeptide synthesizer employing the solid phase method. See also, U.S. Pat. Nos. 5,807,715; 4,816,567; and 6,331,415.

In another alternative, the antibodies can be made recombinantly using procedures that are well known in the art. In one embodiment, a polynucleotide comprises a sequence encoding the heavy chain and/or the light chain variable regions of antibody G1 shown in SEQ ID NO:9 and SEQ ID NO:10. In another embodiment, the polynucleotide comprising the nucleotide sequence shown in SEQ ID NO:9 and SEQ ID NO:10 are cloned into one or more vectors for expression or propagation. The sequence encoding the antibody of interest may be maintained in a vector in a host cell and the host cell can then be expanded and frozen for future use. Vectors (including expression vectors) and host cells are further described herein.

In some embodiments, the invention also encompasses single chain variable region fragments (“scFv”) of antibodies of this invention, such as G1. Single chain variable region fragments are made by linking light and/or heavy chain variable regions by using a short linking peptide. Bird et al. (1988) Science 242:423-426. An example of a linking peptide is (GGGGS)3 (SEQ ID NO:57) which bridges approximately 3.5 nm between the carboxy terminus of one variable region and the amino terminus of the other variable region. Linkers of other sequences have been designed and used. Bird et al. (1988). Linkers can in turn be modified for additional functions, such as attachment of drugs or attachment to solid supports. The single chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad Sci. USA 90:6444-6448; Poljak, R. J., et al. (1994) Structure 2:1121-1123).

For example, bispecific antibodies, monoclonal antibodies that have binding specificities for at least two different antigens, can be prepared using the antibodies disclosed herein. Methods for making bispecific antibodies are known in the art (see, e.g., Suresh et al., 1986, Methods in Enzymology 121:210). Traditionally, the recombinant production of bispecific antibodies was based on the coexpression of two immunoglobulin heavy chain-light chain pairs, with the two heavy chains having different specificities (Millstein and Cuello, 1983, Nature 305, 537-539).

According to one approach to making bispecific antibodies, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2 and CH3 regions. It is preferred to have the first heavy chain constant region (CH1), containing the site necessary for light chain binding, present in at least one of the fusions. DNAs encoding the immunoglobulin heavy chain fusions and, if desired, the immunoglobulin light chain, are inserted into separate expression vectors, and are cotransfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In one approach, the bispecific antibodies are composed of a hybrid immunoglobulin heavy chain with a first binding specificity in one arm, and a hybrid immunoglobulin heavy chain-light chain pair (providing a second binding specificity) in the other arm. This asymmetric structure, with an immunoglobulin light chain in only one half of the bispecific molecule, facilitates the separation of the desired bispecific compound from unwanted immunoglobulin chain combinations. This approach is described in PCT Publication No. WO 94/04690.

Heteroconjugate antibodies, comprising two covalently joined antibodies, are also within the scope of the invention. Such antibodies have been used to target immune system cells to unwanted cells (U.S. Pat. No. 4,676,980), and for treatment of HIV infection (PCT application publication Nos. WO 91/00360 and WO 92/200373; EP 03089). Heteroconjugate antibodies may be made using any convenient cross-linking methods. Suitable cross-linking agents and techniques are well known in the art, and are described in U.S. Pat. No. 4,676,980.

Chimeric or hybrid antibodies also may be prepared in vitro using known methods of synthetic protein chemistry, including those involving cross-linking agents. For example, immunotoxins may be constructed using a disulfide exchange reaction or by forming a thioether bond. Examples of suitable reagents for this purpose include iminothiolate and methyl-4-mercaptobutyrimidate.

Humanized antibody comprising one or more CDRs of antibody G1 or its variants shown in Table 6, or one or more CDRs derived from antibody G1 or its variants shown in Table 6 can be made using any methods known in the art. For example, four general steps may be used to humanize a monoclonal antibody.

In some embodiments, the invention encompasses modifications to antibody G1 or its variants shown in Table 6, including functionally equivalent antibodies which do not significantly affect their properties and variants which have enhanced or decreased activity and/or affinity. For example, the amino acid sequence of antibody G1 or its variants shown in Table 6 may be mutated to obtain an antibody with the desired binding affinity to CGRP. Modification of polypeptides is routine practice in the art and need not be described in detail herein. Modification of polypeptides is exemplified in the Examples. Examples of modified polypeptides include polypeptides with conservative substitutions of amino acid residues, one or more deletions or additions of amino acids which do not significantly deleteriously change the functional activity, or use of chemical analogs.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to an epitope tag. Other insertional variants of the antibody molecule include the fusion to the N- or C-terminus of the antibody of an enzyme or a polypeptide which increases the serum half-life of the antibody.

Substitution variants have at least one amino acid residue in the antibody molecule removed and a different residue inserted in its place. The sites of greatest interest for substitutional mutagenesis include the hypervariable regions, but FR alterations are also contemplated. Conservative substitutions are shown in Table 1 under the heading of “conservative substitutions”. If such substitutions result in a change in biological activity, then more substantial changes, denominated “exemplary substitutions” in Table 1, or as further described below in reference to amino acid classes, may be introduced and the products screened.

TABLE 1
Amino Acid Substitutions

| Original | Conservative | Exemplary |
|---|---|---|
| Residue | Substitutions | Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; |
| | | Norleucine |
| Leu (L) | Ile | Norleucine; Ile; Val; Met; Ala; |
| | | Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; Phe; Ala; |
| | | Norleucine |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) Non-polar: Norleucine, Met, Ala, Val, Leu, Ile;
(2) Polar without charge: Cys, Ser, Thr, Asn, Gln;
(3) Acidic (negatively charged): Asp, Glu;
(4) Basic (positively charged): Lys, Arg;
(5) Residues that influence chain orientation: Gly, Pro; and
(6) Aromatic: Trp, Tyr, Phe, His.

Non-conservative substitutions are made by exchanging a member of one of these classes for another class.

Any cysteine residue not involved in maintaining the proper conformation of the antibody also may be substituted, generally with serine, to improve the oxidative stability of the molecule and prevent aberrant cross-linking. Conversely, cysteine bond(s) may be added to the antibody to improve its stability, particularly where the antibody is an antibody fragment such as an Fv fragment.

Amino acid modifications can range from changing or modifying one or more amino acids to complete redesign of a region, such as the variable region. Changes in the variable region can alter binding affinity and/or specificity. In some embodiments, no more than one to five conservative amino acid substitutions are made within a CDR domain. In other embodiments, no more than one to three conservative amino acid substitutions are made within a CDR domain. In still other embodiments, the CDR domain is CDR H3 and/or CDR L3.

Modifications also include glycosylated and nonglycosylated polypeptides, as well as polypeptides with other post-translational modifications, such as, for example, glycosylation with different sugars, acetylation, and phosphorylation. Antibodies are glycosylated at conserved positions in their constant regions (Jefferis and Lund, 1997, Chem. Immunol. 65:111-128; Wright and Morrison, 1997, TibTECH 15:26-32). The oligosaccharide side chains of the immunoglobulins affect the protein's function (Boyd et al., 1996, Mol. Immunol. 32:1311-1318; Wittwe and Howard, 1990, Biochem. 29:4175-4180) and the intramolecular interaction between portions of the glycoprotein, which can affect the conformation and presented three-dimensional surface of the glycoprotein (Hefferis and Lund, supra; Wyss and Wagner, 1996, Current Opin. Biotech. 7:409-416). Oligosaccharides may also serve to target a given glycoprotein to certain molecules based upon specific recognition structures. Glycosylation of antibodies has also been reported to affect antibody-dependent cellular cytotoxicity (ADCC). In particular, CHO cells with tetracycline-regulated expression of β(1,4)-N-acetylglucosaminyltransferase III (GnTIII), a glycosyltransferase catalyzing formation of bisecting GlcNAc, was reported to have improved ADCC activity (Umana et al., 1999, Mature Biotech. 17:176-180).

Glycosylation of antibodies is typically either N-linked or O-linked. N-linked refers to the attachment of the carbohydrate moiety to the side chain of an asparagine residue. The tripeptide sequences asparagine-X-serine, asparagine-X-threonine, and asparagine-X-cysteine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Other methods of modification include using coupling techniques known in the art, including, but not limited to, enzymatic means, oxidative substitution and chelation. Modifications can be used, for example, for attachment of labels for immunoassay. Modified G1 polypeptides can be made using established procedures in the art and can be screened using standard assays known in the art, some of which are described below and in the Examples.

In some embodiments of the invention, the antibody comprises a modified constant region, such as a constant region that is immunologically inert or partially inert, e.g., does not trigger complement mediated lysis, does not stimulate antibody-dependent cell mediated cytotoxicity (ADCC), or does not activate microglia; or have reduced activities (compared to the unmodified antibody) in any one or more of the following: triggering complement mediated lysis, stimulating antibody-dependent cell mediated cytotoxicity (ADCC), or activating microglia. Different modifications of the constant region may be used to achieve optimal level and/or combination of effector functions. See, for example, Morgan et al., Immunology 86:319-324 (1995); Lund et al., J. Immunology 157:4963-9 157:4963-4969 (1996); Idusogie et al., J. Immunology 164:4178-4184 (2000); Tao et al., J. Immunology 143: 2595-2601 (1989); and Jefferis et al., Immunological Reviews 163:59-76 (1998). In some embodiments, the constant region is modified as described in Eur. J. Immunol. (1999) 29:2613-2624; PCT Application No. PCT/GB99/01441; and/or UK Patent Application No. 9809951.8. In other embodiments, the antibody comprises a human heavy chain IgG2 constant region comprising the following mutations: A330P331 to S330S331 (amino acid numbering with reference to the wildtype IgG2 sequence). Eur. J. Immunol. (1999) 29:2613-2624. In still other embodiments, the constant region is aglycosylated for N-linked glycosylation. In some embodiments, the constant region is aglycosylated for N-linked glycosylation by mutating the glycosylated amino acid residue or flanking residues that are part of the N-glycosylation recognition sequence in the constant region. For example, N-glycosylation site N297 may be mutated to A, Q, K, or H. See, Tao et al., J. Immunology 143: 2595-2601 (1989); and Jefferis et al., Immunological Reviews 163:59-76 (1998). In some embodiments, the constant region is aglycosylated for N-linked glycosylation. The constant region may be aglycosylated for N-linked glycosylation enzymatically (such as removing carbohydrate by enzyme PNGase), or by expression in a glycosylation deficient host cell.

Other antibody modifications include antibodies that have been modified as described in PCT Publication No. WO 99/58572, published Nov. 18, 1999. These antibodies comprise, in addition to a binding domain directed at the target molecule, an effector domain having an amino acid sequence substantially homologous to all or part of a constant domain of a human immunoglobulin heavy chain. These antibodies are capable of binding the target molecule without triggering significant complement dependent lysis, or cell-mediated destruction of the target. In some embodiments, the effector domain is capable of specifically binding FcRn and/or FcγRIIb. These are typically based on chimeric domains derived from two or more human immunoglobulin heavy chain C_{H}2 domains. Antibodies modified in this manner are particularly suitable for use in chronic antibody therapy, to avoid inflammatory and other adverse reactions to conventional antibody therapy.

In some embodiments, the invention includes affinity matured embodiments. For example, affinity matured antibodies can be produced by procedures known in the art (Marks et al., 1992, Bio/Technology, 10:779-783; Barbas et al., 1994, Proc Nat. Acad. Sci, USA 91:3809-3813; Schier et al., 1995, Gene, 169:147-155; Yelton et al., 1995, J. Immunol., 155:1994-2004; Jackson et al., 1995, J. Immunol., 154(7):3310-9; Hawkins et al, 1992, J. Mol. Biol., 226:889-896; and WO2004/058184).

In some embodiments, the invention also encompasses fusion proteins comprising one or more fragments or regions from the antibodies (such as G1) or polypeptides of this invention. In one embodiment, a fusion polypeptide is provided that comprises at least 10 contiguous amino acids of the variable light chain region shown in SEQ ID NO:2 (FIG. 5) and/or at least 10 amino acids of the variable heavy chain region shown in SEQ ID NO:1 (FIG. 5). In other embodiments, a fusion polypeptide is provided that comprises at least about 10, at least about 15, at least about 20, at least about 25, or at least about 30 contiguous amino acids of the variable light chain region shown in SEQ ID NO:2 (FIG. 5) and/or at least about 10, at least about 15, at least about 20, at least about 25, or at least about 30 contiguous amino acids of the variable heavy chain region shown in SEQ ID NO:1 (FIG. 5). In another embodiment, the fusion polypeptide comprises a light chain variable region and/or a heavy chain variable region of G1, as shown in SEQ ID NO:2 and SEQ ID NO:1 of FIG. 5. In another embodiment, the fusion polypeptide comprises one or more CDR(s) of G1. In still other embodiments, the fusion polypeptide comprises CDR H3 and/or CDR L3 of antibody G1. For purposes of this invention, an G1 fusion protein contains one or more G1 antibodies and another amino acid sequence to which it is not attached in the native molecule, for example, a heterologous sequence or a homologous sequence from another region. Exemplary heterologous sequences include, but are not limited to a “tag” such as a FLAG tag or a 6His tag (SEQ ID NO:56). Tags are well known in the art.

In some embodiments, the invention also provides compositions (including pharmaceutical compositions) and kits comprising antibody G1, and/or any or all of the antibodies or polypeptides described herein.

Preferably, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e., the window size) and multiplying the results by 100 to yield the percentage of sequence identity.

Variants may also, or alternatively, be substantially homologous to a native gene, or a portion or complement thereof. Such polynucleotide variants are capable of hybridizing under moderately stringent conditions to a naturally occurring DNA sequence encoding a native antibody (or a complementary sequence).

### D. Compositions

In some embodiments, compositions used in a method of the invention comprise an effective amount of an antibody (e.g., anti-CGRP antagonist antibody, monoclonal antibody that modulates the CGRP pathway) or an antibody derived polypeptide described herein. Examples of such compositions, as well as how to formulate, are also described in an earlier section and below. In one embodiment, the composition further comprises a CGRP antagonist. In some embodiments, the composition comprises one or more monoclonal antibodies that modulate the CGRP pathway. In some embodiments, the composition comprises one or more anti-CGRP antagonist antibodies. In some embodiments, the anti-CGRP antagonist antibody recognizes human CGRP. In some embodiments, the anti-CGRP antagonist antibody is humanized. In some embodiments, the anti-CGRP antagonist antibody comprises a constant region that does not trigger an unwanted or undesirable immune response, such as antibody-mediated lysis or ADCC. In some embodiments, the anti-CGRP antagonist antibody comprises one or more CDR(s) of antibody G1 (such as one, two, three, four, five, or, in some embodiments, all six CDRs from G1). In some embodiments, the anti-CGRP antagonist antibody is human.

It is understood that the compositions can comprise more than one antibody (e.g., more than one anti-CGRP antagonist antibody—a mixture of anti-CGRP antagonist antibodies that recognize different epitopes of CGRP). Other exemplary compositions comprise more than one anti-CGRP antagonist antibodies that recognize the same epitope(s), or different species of anti-CGRP antagonist antibodies that bind to different epitopes of CGRP.

A composition can further comprise pharmaceutically acceptable carriers, excipients, or stabilizers (Remington: The Science and practice of Pharmacy 20th Ed. (2000) Lippincott Williams and Wilkins, Ed. K. E. Hoover). Acceptable carriers, excipients, or stabilizers are nontoxic to recipients at the dosages and concentrations employed. A therapeutic formulation of an antibody may comprise one or more pharmaceutically acceptable carriers, excipients or stabilizes with non-limiting examples of such species that include buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids (e.g., at concentrations of 0.1 mM to 100 mM, 0.1 mM to 1 mM, 0.01 mM to 50 mM, 1 mM to 50 mM, 1 mM to 30 mM, 1 mM to 20 mM, 10 mM to 25 mM) such as glycine, glutamine, methionine, asparagine, histidine, arginine, or lysine; monosaccharides, disaccharides, and other carbohydrates including glucose, mannose, or dextrins; chelating agents (e.g., at concentrations of 0.001 mg/mL to 1 mg/mL, 0.001 mg/mL to 1 mg/mL, 0.001 mg/mL to 0.1 mg/mL, 0.001 mg/mL to 0.01 mg/mL) such as EDTA (e.g., disodium EDTA dihydrate); sugars (e.g., at concentrations of 1 mg/mL to 500 mg/mL, 10 mg/mL to 200 mg/mL, 10 mg/mL to 100 mg/mL, 50 mg/mL to 150 mg/mL) such as sucrose, mannitol, trehalose or sorbitol; salt-forming counter-ions such as sodium; metal complexes (e.g., Zn-protein complexes); and/or non-ionic surfactants (e.g., at concentrations of 0.01 mg/mL to 10 mg/mL, 0.01 mg/mL to 1 mg/mL, 0.1 mg/mL to 1 mg/mL, 0.01 mg/mL to 0.5 mg/mL) such as TWEEN™ (e.g., polysorbate (e.g., polysorbate 20, polysorbate 40, polysorbate 60, polysorbate 80)), PLURONICS™ or polyethylene glycol (PEG). Pharmaceutically acceptable excipients are further described herein.

An antibody (e.g., an anti-CGRP antagonist antibody) and compositions thereof can also be used in conjunction with other agents that serve to enhance and/or complement the effectiveness of the agents.

### E. Kits

In one aspect, the invention also provides kits for use in the instant methods. Kits can include one or more containers comprising an antibody described herein (e.g., an anti-CGRP antagonist antibody (such as a humanized antibody)) or polypeptide described herein and instructions for use in accordance with any of the methods described herein. Generally, these instructions comprise a description of administration of the antibody to treat, ameliorate or prevent CCH or ECH according to any of the methods described herein. The kit may further comprise a description of selecting an individual suitable for treatment based on identifying whether that individual has CCH or ECH or whether the individual is at risk of having CCH or ECH. In still other embodiments, the instructions comprise a description of administering an antibody (e.g., anti-CGRP antagonist antibody) to an individual at risk of having CCH or ECH.

In some embodiments, the antibody is a humanized antibody. In some embodiments, the antibody is human. In other embodiments, the antibody is a monoclonal antibody. In some embodiments, the antibody comprises one or more CDR(s) of antibody G1 (such as one, two, three, four, five, or, in some embodiments, all six CDRs from G1).

The instructions relating to the use of an antibody (e.g., anti-CGRP antagonist antibody) generally include information as to dosage, dosing schedule, and route of administration for the intended treatment. The containers may be unit doses, bulk packages (e.g., multi-dose packages) or sub-unit doses. Instructions supplied in the kits are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, ameliorating and/or preventing CCH or ECH. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an anti-CGRP antagonist antibody and/or a monoclonal antibody that modulates the CGRP pathway. The container may further comprise a second pharmaceutically active agent.

Kits may optionally provide additional components such as buffers and interpretive information. Normally, the kit comprises a container and a label or package insert(s) on or associated with the container.

The following Examples are provided to illustrate but not limit the invention. It is understood that the examples and embodiments described herein are for illustrative purposes only and that various modifications or changes in light thereof will be suggested to persons skilled in the art and are to be included within the spirit and purview of this application. All publications, patents, and patent applications cited herein are hereby incorporated by reference in their entirety for all purposes to the same extent as if each individual publication, patent or patent application were specifically and individually indicated to be so incorporated by reference.

### EXAMPLES

### Example 1: Generation and Characterization of Monoclonal Antibodies Directed Against CGRP

Generation of anti-CGRP antibodies. To generate anti-CGRP antibodies that have cross-species reactivity for rat and human CGRP, mice were immunized with 25-100 μg of human α-CGRP or β-CGRP conjugated to KLH in adjuvant (50 μl per footpad, 100 μl total per mouse) at various intervals. Immunization was generally performed as described in Geerligs H J et al., 1989, J. Immunol. Methods 124:95-102; Kenney J S et al., 1989, J. Immunol. Methods 121:157-166; and Wicher K et al., 1989, Int. Arch. Allergy Appl. Immunol. 89:128-135. Mice were first immunized with 50 μg of human α-CGRP or β-CGRP conjugated to KLH in CFA (complete Freund's adjuvant). After 21 days, mice were secondly immunized with 25 μg of human β-CGRP (for mice first immunized with human α-CGRP) or α-CGRP (for mice first immunized with human β-CGRP) conjugated to KLH in IFA (incomplete Freund's adjuvant). Twenty-three days later after the second immunization, third immunization was performed with 25 μg of rat α-CGRP conjugated to KLH in IFA. Ten days later, antibody titers were tested using ELISA. Forth immunization was performed with 25 μg of the peptide (rat α-CGRP-KLH) in IFA 34 days after the third immunization. Final booster was performed with 100 μg soluble peptide (rat α-CGRP) 32 days after the forth immunization.

Splenocytes were obtained from the immunized mouse and fused with NSO myeloma cells at a ratio of 10:1, with polyethylene glycol 1500. The hybrids were plated out into 96-well plates in DMEM containing 20% horse serum and 2-oxaloacetate/pyruvate/insulin (Sigma), and hypoxanthine/aminopterin/thymidine selection was begun. On day 8, 100 μl of DMEM containing 20% horse serum was added to all the wells. Supernatants of the hybrids were screened by using antibody capture immunoassay. Determination of antibody class was done with class-specific second antibodies.

A panel of monoclonal antibody-producing cell lines was selected based on their binding to human and rat CGRP for further characterization. These antibodies and characteristics are shown below in Tables 2 and 3.

Purification and Fab fragment preparation. Monoclonal antibodies selected for further characterization were purified from supernatants of hybridoma cultures using protein A affinity chromatography. The supernatants were equilibrated to pH 8. The supernatants were then loaded to the protein A column MabSelect (Amersham Biosciences #17-5199-02) equilibrated with PBS to pH 8. The column was washed with 5 column volumes of PBS, pH 8. The antibodies were eluted with 50 mM citrate-phosphate buffer, pH 3. The eluted antibodies were neutralized with 1 M Phosphate Buffer, pH 8. The purified antibodies were dialyzed with PBS, pH 7.4. The antibody concentrations were determined by SDS-PAGE, using a murine monoclonal antibody standard curve.

Fabs were prepared by papain proteolysis of the full antibodies using Immunopure Fab kit (Pierce #44885) and purified by flow through protein A chromatography following manufacturer instructions. Concentrations were determined by ELISA and/or SDS-PAGE electrophoresis using a standard Fab of known concentration (determined by amino acid analysis), and by A280 using 1OD=0.6 mg/ml (or theoretical equivalent based on the amino acid sequence).

Affinity determination of the Fabs. Affinities of the anti-CGRP monoclonal antibodies were determined at either 25° C. or 37° C. using the BIACORE3000™ surface plasmon resonance (SPR) system (Biacore, INC, Piscataway N.J.) with the manufacture's own running buffer, HBS-EP (10 mM HEPES pH 7.4, 150 mM NaCl, 3 mM EDTA, 0.005% v/v polysorbate P20). Affinity was determined by capturing N-terminally biotinylated CGRP peptides (custom ordered from GenScript Corporation, New Jersey or Global Peptide Services, Colorado) via pre-immobilized streptavidin on SA chip and measuring binding kinetics of antibody Fab titrated across the CGRP surface. Biotinylated CGRP was diluted into HBS-EP and injected over the chip at a concentration of less than 0.001 mg/ml. Using variable flow time across the individual chip channels, two ranges of antigen density were achieved: <50 response units (RU) for detailed kinetic studies and about 800 RU for concentration studies and screening. Two- or three-fold serial dilutions typically at concentrations spanning 1 μM-0.1 nM (aimed at 0.1-10× estimated K_{D}) of purified Fab fragments were injected for 1 minute at 100 μL/min and dissociation times of 10 minutes were allowed. After each binding cycle, surfaces were regenerated with 25 mM NaOH in 25% v/v ethanol, which was tolerated over hundreds of cycles. Kinetic association rate (kₒₙ) and dissociation rate (k_{off}) were obtained simultaneously by fitting the data to a 1:1 Langmuir binding model (Karlsson, R. Roos, H. Fagerstam, L. Petersson, B. (1994). Methods Enzymology 6. 99-110) using the BIAevaluation program. Global equilibrium dissociation constants (K_{D}) or “affinities” were calculated from the ratio K_{D}=k_{off}/kₒₙ. Affinities of the murine Fab fragments are shown in Tables 2 and 3.

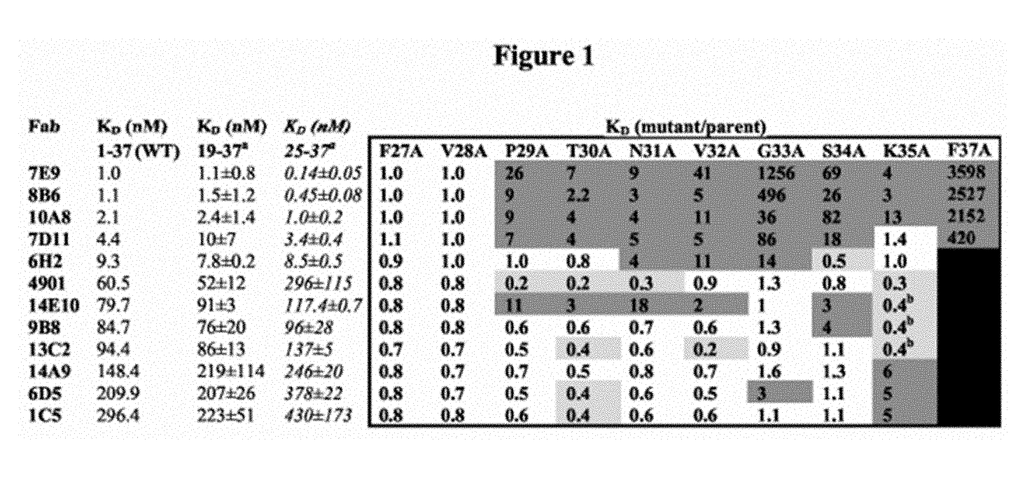

Epitope mapping of the murine anti-CGRP antibodies. To determine the epitope that anti-CGRP antibodies bind on human α-CGRP, binding affinities of the Fab fragments to various CGRP fragments were measured as described above by capturing N-terminally biotinylated CGRP fragments amino acids 19-37 and amino acids 25-37 on a SA sensor chip. FIG. 1 shows their binding affinities measured at 25° C. As shown in FIG. 1, all antibodies, except antibody 4901, bind to human α-CGRP fragments 19-37 and 25-37 with affinity similar to their binding affinity to full length human α-CGRP (1-37). Antibody 4901 binds to human α-CGRP fragment 25-37 with six-fold lower affinity than binding to full length human α-CGRP fragment, due mainly to a loss in off-rate. The data indicate that these anti-CGRP antibodies generally bind to the C-terminal end of CGRP.

Alanine scanning was performed to further characterize amino acids in human α-CGRP involved in binding of anti-CGRP antibodies. Different variants of human α-CGRP with single alanine substitutions were generated by peptide synthesis. Their amino acid sequences are shown in Table 4 along with all the other peptides used in the Biacore analysis. Affinities of Fab fragments of the anti-CGRP antibodies to these variants were determined using Biacore as described above. As shown in FIG. 1, all 12 antibodies target a C-terminal epitope, with amino acid F37 being the most crucial residue. Mutation of F37 to alanine significantly lowered the affinity or even completely knocked out binding of the anti-CGRP antibodies to the peptide. The next most important amino acid residue is G33, however, only the high affinity antibodies (7E9, 8B6, 10A8, and 7D11) were affected by alanine replacement at this position. Amino acid residue S34 also plays a significant, but lesser, role in the binding of these four high affinity antibodies.

TABLE 2
Characteristics of the anti-CGRP monoclonal antibodies' binding to human
α-CGRP and their antagonist activity

| | | | Cell-based blocking | IC₅₀ (nM binding |
|---|---|---|---|---|
| | | | human α-CGRP | sites) at 25° C. |
| | | | binding to its | (room temp.) |
| | K_{D} to human α- | K_{D} to human α- | receptor at 25° C. | measured in |
| | CGRP at 25° C. | CGRP at 37° C. | (measured by cAMP | radioligand binding |
| Antibodies | (nM) | (nM) | activation) | assay. |
| 7E9 | 1.0 | 0.9 | Yes | 2.5 |
| 8B6 | 1.1 | 1.2 | Yes | 4.0 |
| 10A8 | 2.1 | 3.0 | Yes | n.d. |
| 7D11 | 4.4 | 5.4 | Yes | n.d. |
| 6H2 | 9.3 | 42 | Yes | 12.9 |
| 4901 | 61 | 139 | Yes | 58 |
| 14E10 | 80 | 179 | Yes | n.d. |
| 9B8 | 85 | 183 | No | n.d. |
| 13C2 | 94 | 379 | No | n.d. |
| 14A9 | 148 | 581 | No | n.d. |
| 6D5 | 210 | 647 | No | n.d. |
| 1C5 | 296 | 652 | No | n.d. |

Note:
Antibody 4901 is commercially available (Sigma, Product No. C7113).
n.d. = not determined

TABLE 3
Characteristics of the anti-CGRP monoclonal
antibodies' binding to rat α-CGRP and antagonist activity

| | | Cell-based blocking of | |
|---|---|---|---|
| | | binding of rat α-CGRP | |
| | K_{D} to | to its receptor at 25° C. | In vivo blocking |
| | rat α-CGRP at | (measured by cAMP | in saphenous |
| Antibodies | 37° C. (nM) | activation) | nerve assay |
| 4901 | 3.4 | Yes | Yes |
| 7E9 | 47 | Yes | Yes |
| 6H2 | 54 | No | No |
| 8B6 | 75 | Yes | Yes |
| 7D11 | 218 | Yes | Yes |
| 10A8 | 451 | No | n.d. |
| 9B8 | 876 | No | n.d. |
| 14E10 | 922 | No | n.d. |
| 13C2 | >1000 | No | n.d. |
| 14A9 | >1000 | No | n.d. |
| 6D5 | >1000 | No | n.d. |
| 1C5 | >1000 | No | n.d. |

“n.d.” indicates no test was performed for the antibody.

TABLE 4
Amino acid sequences of human α-CGRP fragments
(SEQ ID NOS: 15-40) and related peptides (SEQ
ID NOS: 41-47). All peptides are C-terminally
amidated except SEQ ID NOS: 36-40. Residues in
bold indicate point mutations.

| | | SEQ |
|---|---|---|
| | | ID |
| CGRP | Amino acid sequence | NO |
| 1-37 | ACDTATCVTHRLAGLLSRSGGVVKNNFVPTNVGSKAF | 15 |
| (WT) | | |
| 8-37 | VTHRLAGLLSRSGGVVKNNFVPTNVGSKAF | 16 |
| 19-37 | SGGVVKNNFVPTNVGSKAF | 17 |
| P29A | SGGVVKNNFVATNVGSKAF | 18 |
| (19-37) | | |
| K35A | SGGVVKNNFVPTNVGSAAF | 19 |
| (19-37) | | |
| K35E | SGGVVKNNFVPTNVGSEAF | 20 |
| (19-37) | | |
| K35M | SGGVVKNNFVPTNVGSMAF | 21 |
| (19-37) | | |
| K35Q | SGGVVKNNFVPTNVGSQAF | 22 |
| (19-37) | | |
| F37A | SGGVVKNNFVPTNVGSKAA | 23 |
| (19-37) | | |
| 25-38A | NNFVPTNVGSKAFA | 24 |
| 25-37 | NNFVPTNVGSKAF | 25 |
| F27A | NNAVPTNVGSKAF | 26 |
| (25-37) | | |
| V28A | NNFAPTNVGSKAF | 27 |
| (25-37) | | |
| P29A | NNFVATNVGSKAF | 28 |
| (25-37) | | |
| T30A | NNFVPANVGSKAF | 29 |
| (25-37) | | |
| N31A | NNFVPTAVGSKAF | 30 |
| (25-37) | | |
| V32A | NNFVPTNAGSKAF | 31 |
| (25-37) | | |
| G33A | NNFVPTNVASKAF | 32 |
| (25-37) | | |
| S34A | NNFVPTNVGAKAF | 33 |
| (25-37) | | |
| F37A | NNFVPTNVGSKAA | 34 |
| (25-37) | | |
| 26-37 | NFVPTNVGSKAF | 35 |
| 19-37- | SGGVVKNNFVPTNVGSKAF | 36 |
| COOH | | |
| 19-36- | SGGVVKNNFVPTNVGSKA | 37 |
| COOH | | |
| 1-36-COOH | ACDTATCVTHRLAGLLSRSGGVVKNNFVPTNVGSKA | 38 |
| 1-19-COOH | ACDTATCVTHRLAGLLSRS | 39 |
| 1-13-COOH | ACDTATCVTHRLA | 40 |
| rat α | SCNTATCVTHRLAGLLSRSGGVVKDNFVPTNVGSEAF | 41 |
| (1-37) | | |
| rat α | SGGVVKDNFVPTNVGSEAF | 42 |
| (19-37) | | |
| human β | ACNTATCVTHRLAGLLSRSGGMVKSNFVPTNVGSKAF | 43 |
| (1-37) | | |
| rat β | SCNTATCVTHRLAGLLSRSGGVVKDNFVPTNVGSKAF | 44 |
| (1-37) | | |
| Human | CGNLSTCMLGTYTQDFNKFHTFPQTAIGVGAP | 45 |
| calci- | | |
| tonin | | |
| (1-32) | | |
| Human | KCNTATCATQRLANFLVHSSNNFGAILSSTNVGSNTY | 46 |
| amylin | | |
| (1-37) | | |
| Human | YRQSMNNFQGLRSFGCRFGTCTVQKLAHQIYQFTDK | 47 |
| adreno- | DKDNVAPRSKISPQGY | |
| medullin | | |
| (1-52) | | |

### Example 2: Screening of Anti-CGRP Antagonist Antibodies Using In Vitro Assays

Murine anti-CGRP antibodies were further screened for antagonist activity in vitro using cell based cAMP activation assay and binding assay.

Antagonist activity measured by cAMP assay. Five microliters of human or rat α-CGRP (final concentration 50 nM) in the presence or absence of an anti-CGRP antibody (final concentration 1-3000 nM), or rat α-CGRP or human α-CGRP (final concentration 0.1 nM-10 μM; as a positive control for c-AMP activation) was dispensed into a 384-well plate (Nunc, Cat. No. 264657). Ten microliters of cells (human SK-N-MC if human α-CGRP is used, or rat L6 from ATCC if rat α-CGRP is used) in stimulation buffer (20 mM HEPES, pH 7.4, 146 mM NaCl, 5 mM KCl, 1 mM CaCl₂, 1 mM MgCl₂, and 500 μM 3-Isobutyl-1-methylxanthine (IBMX)) were added into the wells of the plate. The plate was incubated at room temperature for 30 minutes.

After the incubation, cAMP activation was performed using HitHunter™ Enzyme Fragment Complementation Assay (Applied Biosystems) following manufacture's instruction. The assay is based on a genetically engineered β-galactosidase enzyme that consists of two fragments—termed Enzyme Acceptor (EA) and Enzyme Donor (ED). When the two fragments are separated, the enzyme is inactive. When the fragments are together they can recombine spontaneously to form active enzyme by a process called complementation. The EFC assay platform utilizes an ED-cAMP peptide conjugate in which cAMP is recognized by anti-cAMP. This ED fragment is capable of reassociation with EA to form active enzyme. In the assay, anti-cAMP antibody is optimally titrated to bind ED-cAMP conjugate and inhibit enzyme formation. Levels of cAMP in cell lysate samples compete with ED-cAMP conjugate for binding to the anti-cAMP antibody. The amount of free ED conjugate in the assay is proportional to the concentration of cAMP. Therefore, cAMP is measured by the formation of active enzyme that is quantified by the turnover of β-galactosidase luminescent substrate. The cAMP activation assay was performed by adding 10 μl of lysis buffer and anti-cAMP antibody (1:1 ratio) following by incubation at room temperature for 60 min. Then 10 μl of ED-cAMP reagent was added into each well and incubated for 60 minutes at room temperature. After the incubation, 20 μl of EA reagent and CL mixture (containing the substrate) (1:1 ratio) was added into each well and incubated for 1-3 hours or overnight at room temperature. The plate was read at 1 second/well on PMT instrument or 30 seconds/place on imager. The antibodies that inhibit activation of cAMP by α-CGRP were identified (referred to as “yes”) in Tables 2 and 3 above. Data in Tables 2 and 3 indicate that antibodies that demonstrated antagonist activity in the assay generally have high affinity. For example, antibodies having K_{D} (determined at 25° C.) of about 80 nM or less to human α-CGRP or having K_{D} (determined at 37° C.) of about 47 nM or less to rat α-CGRP showed antagonist activity in this assay.

Radioligand binding assay. Binding assay was performed to measure the IC₅₀ of anti-CGRP antibody in blocking the CGRP from binding to the receptor as described previously. Zimmermann et al., Peptides 16:421-4, 1995; Mallee et al., J. Biol. Chem. 277:14294-8, 2002. Membranes (25 μg) from SK-N-MC cells were incubated for 90 min at room temperature in incubation buffer (50 mM Tris-HCl, pH 7.4, 5 mM MgCl₂, 0.1% BSA) containing 10 pM ¹²⁵I-human α-CGRP in a total volume of 1 mL. To determine inhibition concentrations (IC₅₀), antibodies or unlabeled CGRP (as a control), from a about 100 fold higher stock solution were dissolved at varying concentrations in the incubation buffer and incubated at the same time with membranes and 10 pM ¹²⁵I-human α-CGRP. Incubation was terminated by filtration through a glass microfiber filter (GF/B, 1 μm) which had been blocked with 0.5% polyethylemimine. Dose response curves were plotted and K values were determined by using the equation: Kᵢ=IC₅₀/(1+([ligand]/K_{D}); where the equilibrium dissociation constant K_{D}=8 pM for human α-CGRP to CGRP1 receptor as present in SK-N-MC cells, and Bₘₐₓ=0.025 pmol/mg protein. The reported IC₅₀ value (in terms of IgG molecules) was converted to binding sites (by multiplying it by 2) so that it could be compared with the affinities (K_{D}) determined by Biacore (see Table 2).

Table 2 shows the IC₅₀ of murine antibodies 7E9, 8B6, 6H2 and 4901. Data indicate that antibody affinity generally correlates with IC₅₀: antibodies with higher affinity (lower K_{D} values) have lower IC₅₀ in the radioligand binding assay.

### Example 3: Effect of Anti-CGRP Antagonist Antibodies on Skin Vasodilatation Induced by Stimulation of Rat Saphenous Nerve

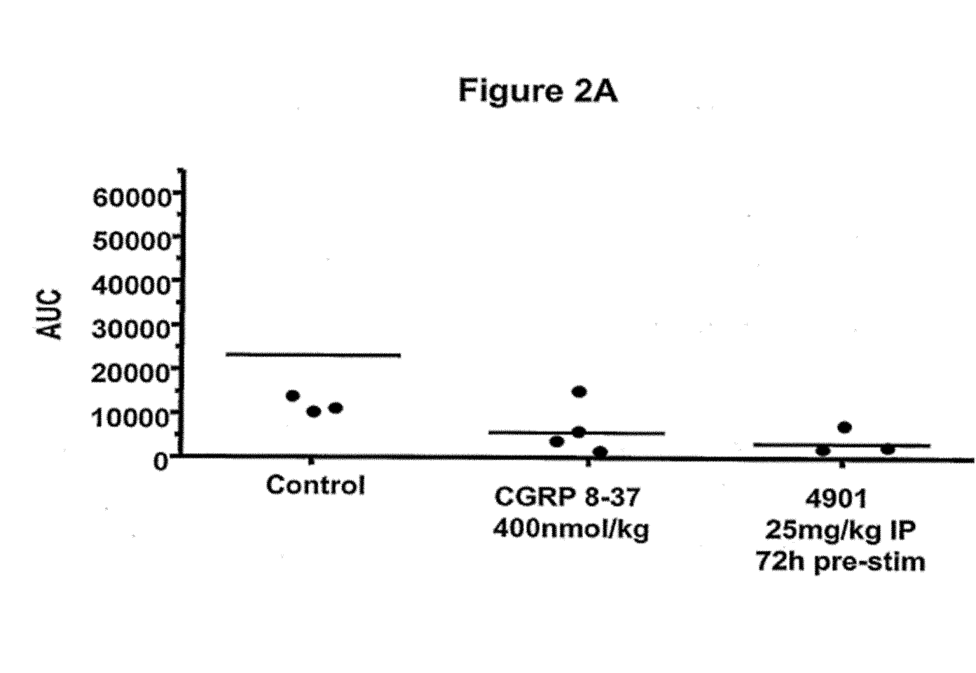

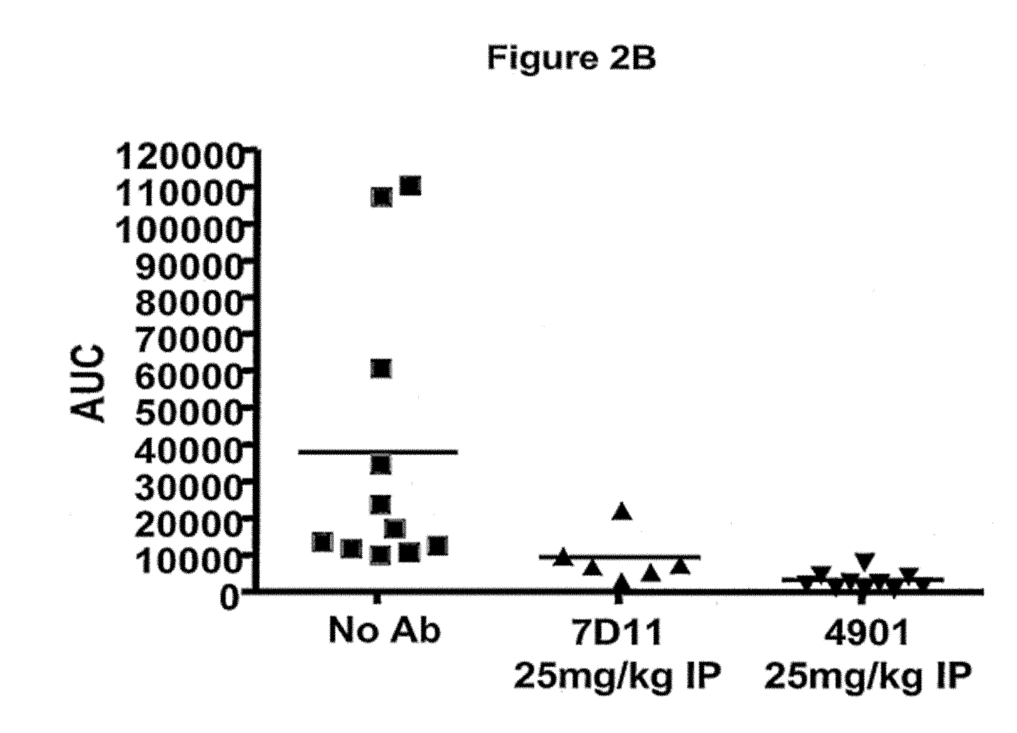

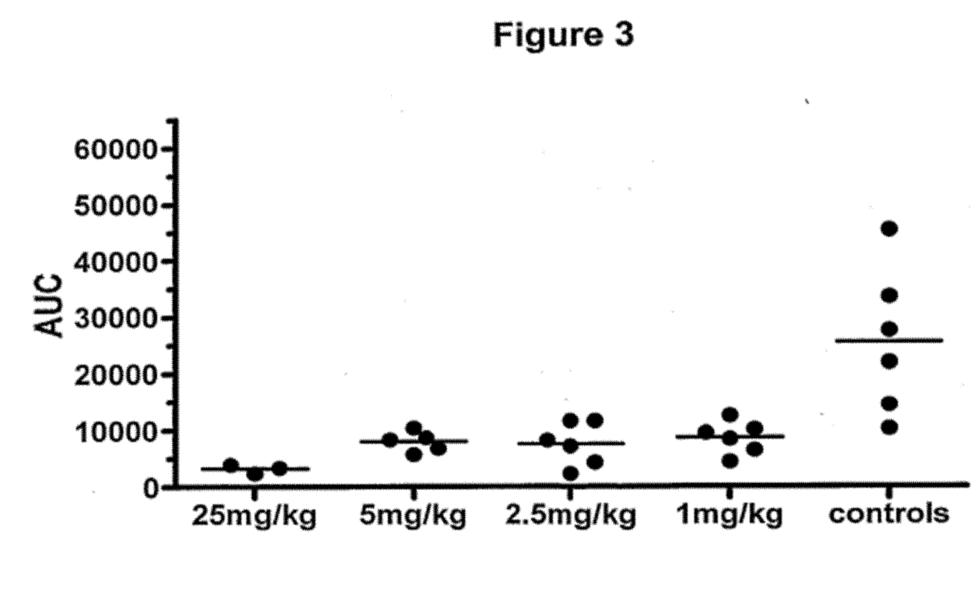

To test antagonist activity of anti-CGRP antibodies, effect of the antibodies on skin vasodilatation by stimulation of rat saphenous nerve was tested using a rat model described previously. Escott et al., Br. J. Pharmacol. 110:772-776, 1993. In this rat model, electrical stimulation of saphenous nerve induces release of CGRP from nerve endings, resulting in an increase in skin blood flow. Blood flow in the foot skin of male Sprague Dawley rats (170-300 g, from Charles River Hollister) was measured after saphenous nerve stimulation. Rats were maintained under anesthesia with 2% isoflurane. Bretylium tosylate (30 mg/kg, administered i.v.) was given at the beginning of the experiment to minimize vasoconstriction due to the concomitant stimulation of sympathetic fibers of the saphenous nerve. Body temperature was maintained at 37° C. by the use of a rectal probe thermostatically connected to a temperature controlled heating pad. Compounds including antibodies, positive control (CGRP 8-37), and vehicle (PBS, 0.01% Tween 20) were given intravenously through the right femoral vein, except for the experiment shown in FIG. 3, the test compound and the control were injected through tail vein, and for experiments shown in FIGS. 2A and 2B, antibodies 4901 and 7D11 were injected intraperitoneally (IP). Positive control compound CGRP 8-37 (vasodilatation antagonist), due to its short half-life, was given 3-5 min before nerve stimulation at 400 nmol/kg (200 μl). Tan et al., Clin. Sci. 89:656-73, 1995. The antibodies were given in different doses (1 mg/kg, 2.5 mg/kg, 5 mg/kg, 10 mg/kg, and 25 mg/kg).

For experiments shown in FIGS. 2A and 2B, antibody 4901 (25 mg/kg), antibody 7D11 (25 mg/kg), or vehicle control (PBS with 0.01% Tween 20) was administered intraperitoneally (IP) 72 hours before the electrical pulse stimulation. For experiment shown in FIG. 3, antibody 4901 (1 mg/kg, 2.5 mg/kg, 5 mg/kg, or 25 mg/kg) or vehicle control (PBS with 0.01% Tween 20) was administered intravenously 24 hours before the electrical pulse stimulation. After administration of the antibodies or vehicle control, the saphenous nerve of the right hindlimb was exposed surgically, cut proximally and covered with plastic wrap to prevent drying. A laser Doppler probe was placed over the medio-dorsal side of the hindpaw skin, which is the region innervated by the saphenous nerve. Skin blood flow, measured as blood cell flux, was monitored with a laser Doppler flow meter. When a stable base-line flux (less than 5% variation) was established for at least 5 minutes, the nerve was placed over platinum bipolar electrodes and electrically stimulated with 60 pulses (2 Hz, 10 V, 1 ms, for 30 seconds) and then again 20 minutes later. Cumulative change in skin blood flow was estimated by the area under the flux-time curve (AUC, which is equal to change in flux multiplied by change in time) for each flux response to electrical pulse stimulation. The average of the blood flow response to the two stimulations was taken. Animals were kept under anesthesia for a period of one to three hours.

As shown in FIG. 2A and FIG. 2B, blood flow increase stimulated by applying electronic pulses on saphenous nerve was inhibited by the presence of CGRP 8-37 (400 nmol/kg, administered i.v.), antibody 4901 (25 mg/kg, administered ip), or antibody 7D11 (25 mg/kg, administered ip) as compared to the control. CGRP 8-37 was administered 3-5 minutes before the saphenous nerve stimulation; and antibodies were administered 72 hours before the saphenous nerve stimulation. As shown in FIG. 3, blood flow increase stimulated by applying electronic pulses on saphenous nerve was inhibited by the presence of antibody 4901 at different doses (1 mg/kg, 2.5 mg/kg, 5 mg/kg, and 25 mg/kg) administered intravenously at 24 hours before the saphenous nerve stimulation.

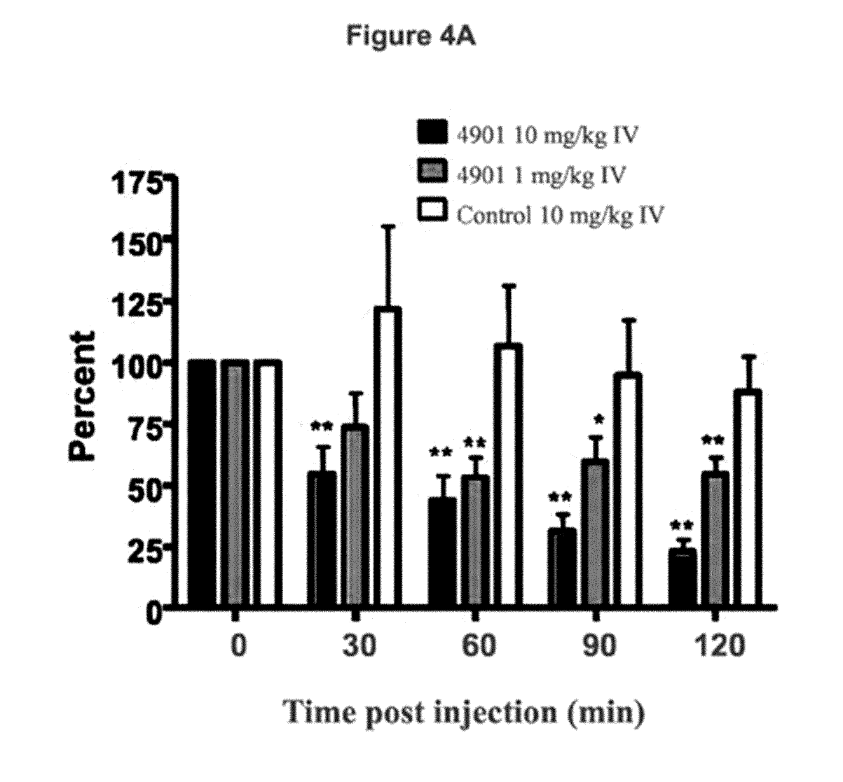

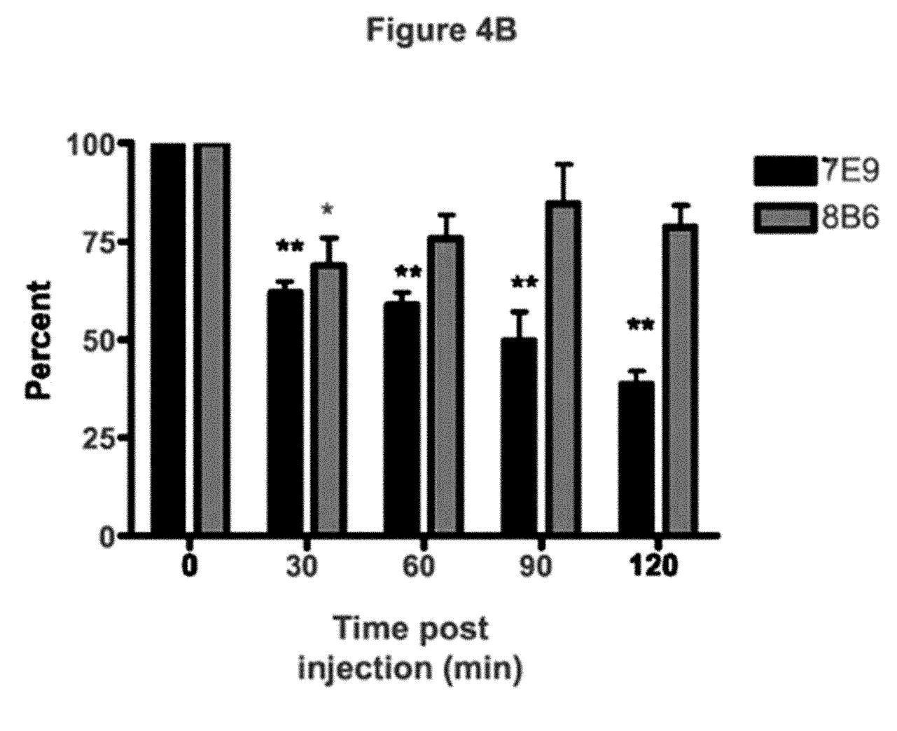

For experiments shown in FIGS. 4A and 4B, saphenous nerve was exposed surgically before antibody administration. The saphenous nerve of the right hindlimb was exposed surgically, cut proximally and covered with plastic wrap to prevent drying. A laser Doppler probe was placed over the medio-dorsal side of the hindpaw skin, which is the region innervated by the saphenous nerve. Skin blood flow, measured as blood cell flux, was monitored with a laser Doppler flow meter. Thirty to forty-five minutes after bretylium tosylate injection, when a stable base-line flux (less than 5% variation) was established for at least 5 minutes, the nerve was placed over platinum bipolar electrodes and electrically stimulated (2 Hz, 10V, 1 ms, for 30 seconds) and again 20 minutes later. The average of the blood flow flux response to these two stimulations was used to establish the baseline response (time 0) to electrical stimulation. Antibody 4901 (1 mg/kg or 10 mg/kg), antibody 7E9 (10 mg/kg), antibody 8B6 (10 mg/kg), or vehicle (PBS with 0.01% Tween 20) were then administered intravenously (i.v.). The nerve was subsequently stimulated (2 Hz, 10V, 1 ms, for 30 sec) at 30 minutes, 60 minutes, 90 minutes, and 120 minutes after antibody or vehicle administration. Animals were kept under anesthesia for a period of approximately three hours. Cumulative change in skin blood flow was estimated by the area under the flux-time curve (AUC, which is equal to change in flux multiplied by change in time) for each flux response to electrical pulse stimulations.

As shown in FIG. 4A, blood flow increase stimulated by applying electronic pulses on saphenous nerve was significantly inhibited by the presence of antibody 4901 1 mg/kg administered i.v., when electronic pulse stimulation was applied at 60 minutes, 90 minutes, and 120 minutes after the antibody administration, and blood flow increase stimulated by applying electronic pulses on saphenous nerve was significantly inhibited by the presence of antibody 4901 10 mg/kg administered i.v., when electronic pulse stimulation was applied at 30 minutes, 60 minutes, 90 minutes, and 120 minutes after the antibody administration. FIG. 4B shows that blood flow increase stimulated by applying electronic pulses on saphenous nerve was significantly inhibited by the presence of antibody 7E9 (10 mg/kg, administered i.v.) when electronic pulse stimulation was applied at 30 min, 60 min, 90 min, and 120 min after antibody administration, and by the presence of antibody 8B6 (10 mg/kg, administered i.v.) when electronic pulse stimulation was applied at 30 min after antibody administration.

These data indicate that antibodies 4901, 7E9, 7D11, and 8B6 are effective in blocking CGRP activity as measured by skin vasodilatation induced by stimulation of rat saphenous nerve.

### Example 4. Characterization of Anti-CGRP Antibody G1 and its Variants

Amino acid sequences for the heavy chain variable region and light chain variable region of anti-CGRP antibody G1 are shown in FIG. 5. The following methods were used for expression and characterization of antibody G1 and its variants.

Expression vector used. Expression of the Fab fragment of the antibodies was under control of an IPTG inducible lacZ promoter similar to that described in Barbas (2001) Phage display: a laboratory manual, Cold Spring Harbor, N.Y., Cold Spring Harbor Laboratory Press pg. 2.10. Vector pComb3X), however, modifications included addition and expression of the following additional domains: the human Kappa light chain constant domain and the CH1 constant domain of IgG2 human immunoglobulin, Ig gamma-2 chain C region, protein accession number P01859; Immunoglobulin kappa light chain (Homo sapiens), protein accession number CAA09181.

Small scale Fab preparation. From E. coli transformed (either using electroporation-competent TG1 cells or chemically-competent Top 10 cells) with a Fab library, single colonies were used to inoculate both a master plate (agar LB+carbenicillin (50 μg/mL)+2% glucose) and a working plate (2 mL/well, 96-well/plate) where each well contained 1.5 mL LB+carbenicillin (50 μg/mL)+2% glucose. A gas permeable adhesive seal (ABgene, Surrey, UK) was applied to the plate. Both plates were incubated at 30° C. for 12-16 hours; the working plate was shaken vigorously. The master plate was stored at 4° C. until needed, while the cells from the working plate were pelleted (4000 rpm, 4° C., 20 minutes) and resuspended in 1.0 mL LB+carbenicillin (50 μg/mL)+0.5 mM IPTG to induce expression of Fabs by vigorous shaking for 5 hours at 30° C. Induced cells were centrifuges at 4000 rpm, 4° C. for 20 minutes and resuspended in 0.6 mL Biacore HB-SEP buffer (10 mM HEPES pH 7.4, 150 mM NaCl, 3 mM EDTA, 0.005% v/v P20). Lysis of HB-SEP resuspended cells was accomplished by freezing (−80° C.) and then thawing at 37° C. Cell lysates were centrifuged at 4000 rpm, 4° C. for 1 hour to separate the debris from the Fab-containing supernatants, which were subsequently filtered (0.2 μm) using a Millipore MultiScreen Assay System 96-Well Filtration Plate and vacuum manifold. Biacore was used to analyze filtered supernatants by injecting them across CGRPs on the sensor chip. Affinity-selected clones expressing Fabs were rescued from the master plate, which provided template DNA for PCR, sequencing, and plasmid preparation.

Large scale Fab preparation. To obtain kinetic parameters, Fabs were expressed on a larger scale as follows. Erlenmeyer flasks containing 150 mL LB+carbenicillin (50 μg/mL)+2% glucose were inoculated with 1 mL of a “starter” overnight culture from an affinity-selected Fab-expressing E. coli clone. The remainder of the starter culture (˜3 mL) was used to prepare plasmid DNA (QIAprep mini-prep, Qiagen kit) for sequencing and further manipulation. The large culture was incubated at 30° C. with vigorous shaking until an OD_{600 nm} of 1.0 was attained (typically 12-16 h). The cells were pelleted by centrifuging at 4000 rpm, 4° C. for 20 minutes, and resuspended in 150 mL LB+carbenicillin (50 μg/mL)+0.5 mM IPTG. After 5 hours expression at 30° C., cells were pelleted by centrifuging at 4000 rpm, 4° C. for 20 minutes, resuspended in 10 mL Biacore HBS-EP buffer, and lysed using a single freeze (−80° C.)/thaw (37° C.) cycle. Cell lysates were pelleted by centrifuging at 4000 rpm, 4° C. for one hour, and the supernatant was collected and filtered (0.2 um). Filtered supernatants were loaded onto Ni-NTA superflow sepharose (Qiagen, Valencia, Calif.) columns equilibrated with PBS, pH 8, then washed with 5 column volumes of PBS, pH 8. Individual Fabs eluted in different fractions with PBS (pH 8)+300 mM Imidazole. Fractions containing Fabs were pooled and dialyzed in PBS, then quantified by ELISA prior to affinity characterization.

Full antibody preparation. For expression of full antibodies, heavy and light chain variable regions were cloned in mammalian expression vectors and transfected using lipofectamine into HEK 293 cells for transient expression. Antibodies were purified using protein A using standard methods.

Vector pDb.CGRP.hFcGI is an expression vector comprising the heavy chain of the G1 antibody, and is suitable for transient or stable expression of the heavy chain. Vector pDb.CGRP.hFcGI has nucleotide sequences corresponding to the following regions: the murine cytomegalovirus promoter region (nucleotides 7-612); a synthetic intron (nucleotides 613-1679); the DHFR coding region (nucleotides 688-1253); human growth hormone signal peptide (nucleotides 1899-1976); heavy chain variable region of G1 (nucleotides 1977-2621); human heavy chain IgG2 constant region containing the following mutations: A330P331 to S330S331 (amino acid numbering with reference to the wildtype IgG2 sequence; see Eur. J. Immunol. (1999) 29:2613-2624). Vector pDb.CGRP.hFcGI was deposited at the ATCC on Jul. 15, 2005, and was assigned ATCC Accession No. PTA-6867.

Vector pEb.CGRP.hKGI is an expression vector comprising the light chain of the G1 antibody, and is suitable for transient expression of the light chain. Vector pEb.CGRP.hKGI has nucleotide sequences corresponding to the following regions: the murine cytomegalovirus promoter region (nucleotides 2-613); human EF-1 intron (nucleotides 614-1149); human growth hormone signal peptide (nucleotides 1160-1237); antibody G1 light chain variable region (nucleotides 1238-1558); human kappa chain constant region (nucleotides 1559-1882). Vector pEb.CGRP.hKGI was deposited at the ATCC on Jul. 15, 2005, and was assigned ATCC Accession No. PTA-6866.

Biacore assay for affinity determination. Affinities of G1 monoclonal antibody and its variants were determined at either 25° C. or 37° C. using the BIACORE3000™ surface plasmon resonance (SPR) system (Biacore, INC, Piscataway N.J.). Affinity was determined by capturing N-terminally biotinylated CGRP or fragments via pre-immobilized streptavidin (SA sensor chip) and measuring the binding kinetics of antibody G1 Fab fragments or variants titrated across the CGRP or fragment on the chip. All Biacore assays were conducted in HBS-EP running buffer (10 mM HEPES pH 7.4, 150 mM NaCl, 3 mM EDTA, 0.005% v/v polysorbate P20). CGRP surfaces were prepared by diluting the N-biotinylated CGRP to a concentration of less than 0.001 mg/mL into HBS-EP buffer and injecting it across the SA sensor chip using variable contact times. Low capacity surfaces, corresponding to capture levels <50 response units (RU) were used for high-resolution kinetic studies, whereas high capacity surfaces (about 800 RU of captured CGRP) were used for concentration studies, screening, and solution affinity determinations. Kinetic data were obtained by diluting antibody G1 Fab serially in two- or three-fold increments to concentrations spanning 1 uM-0.1 nM (aimed at 0.1-10× estimated K_{D}). Samples were typically injected for 1 minute at 100 μL/min and dissociation times of at least 10 minutes were allowed. After each binding cycle, surfaces were regenerated with 25 mM NaOH in 25% v/v ethanol, which was tolerated over hundreds of cycles. An entire titration series (typically generated in duplicate) was fit globally to a 1:1 Langmuir binding model using the BIAevaluation program. This returned a unique pair of association and dissociation kinetic rate constants (respectively, kₒₙ and k_{off}) for each binding interaction, whose ratio gave the equilibrium dissociation constant (K_{D}=k_{off}/kₒₙ). Affinities (K_{D} values) determined in this way are listed in Tables 6 and 7.

High-resolution analysis of binding interactions with extremely slow offrates. For interactions with extremely slow offrates (in particular, antibody G1 Fab binding to human α-CGRP on the chip at 25° C.), affinities were obtained in a two-part experiment. The protocol described above was used with the following modifications. The association rate constant (kₒₙ) was determined by injecting a 2-fold titration series (in duplicate) spanning 550 nM-1 nM for 30 seconds at 100 μL/min and allowing only a 30 second dissociation phase. The dissociation rate constant (k_{off}) was determined by injecting three concentrations (high, medium, and low) of the same titration series in duplicate for 30 seconds and allowing a 2-hour dissociation phase. The affinity (K_{D}) of each interaction was obtained by combining the kₒₙ and k_{off} values obtained in both types of experiments, as shown in Table 5.

Determining solution affinity by Biacore. The solution affinity of antibody G1 for rat α-CGRP and F37A (19-37) human α-CGRP was measured by Biacore at 37° C. A high capacity CGRP chip surface was used (the high-affinity human α-CGRP was chosen for detection purposes) and HBS-EP running buffer was flowed at 5 μL/min. Antibody G1 Fab fragment at a constant concentration of 5 nM (aimed to be at or below the expected K_{D} of the solution-based interaction) was pre-incubated with competing peptide, either rat α-CGRP or F37A (19-37) human α-CGRP, at final concentrations spanning 1 nM to 1 μM in 3-fold serial dilutions. Antibody G1 Fab solutions in the absence or presence of solution-based competing peptide, were injected across CGRP on the chip and the depletion of binding responses detected at the chip surface as a result of solution competition was monitored. These binding responses were converted to “free Fab concentrations” using a calibration curve, which was constructed by titrating antibody G1 Fab alone (5, 2.5, 1.25, 0.625, 0.325 and 0 nM) across the CGRP on the chip. “Free Fab concentrations” were plotted against the concentration of competing solution-based peptide used to generate each data point and fit to a solution affinity model using the BIAevaluation software. The solution affinities determined (indirectly) in this way are shown in Tables 5 and 7 and were used to validate the affinities obtained when Fabs are injected directly across N-biotinylated CGRPs on a SA chip. The close agreement between the affinities determined by these two methods confirms that tethering an N-biotinylated version of the CGRP to the chip does not alter its native solution binding activity.

Table 5 below shows the binding affinities of antibody G1 to human α-CGRP, human β-CGRP, rat α-CGRP, and rat β-CGRP determined by Biacore, by flowing Fab fragments across N-biotinylated CGRPs on a SA chip. To better resolve the affinities of binding interactions with extremely slow offrates, affinities were also determined in a two-part experiment to complement this assay orientation, the solution affinity of the rat α-CGRP interaction was also determined (as described above). The close agreement of the affinities measured in both assay orientations confirms that the binding affinity of the native rat α-CGRP in solution is not altered when it is N-biotinylated and tethered to a SA chip.

TABLE 5
Binding affinities of antibody G1 Fabs titrated across CGRPs on the chip

| | Temp. | | | |
|---|---|---|---|---|
| CGRP on chip | (° C.) | kₒₙ (1/Ms) | k_{off} (1/s) | K_{D} (nM) |
| Human | 25 | 1.86 × 10⁵ | 7.80 × 10⁻⁶ | 0.042 (7%, n = 4)* |
| α-CGRP | | | | |
| Human | 37 | 5.78 × 10⁵ | 3.63 × 10⁻⁵ | 0.063 (4%, n = 2)* |
| α-CGRP | | | | |
| Human | 37 | 4.51 × 10⁵ | 6.98 × 10⁻⁵ | 0.155 |
| β-CGRP | | | | |
| Rat α-CGRP | 25 | 5.08 × 10⁴ | 6.18 × 10⁻⁵ | 1.22 (12%, n = 2)* |
| Rat α-CGRP | 37 | 1.55 × 10⁵ | 3.99 × 10⁻⁴ | 2.57* |
| | | | | (Solution K_{D} = 10 |
| | | | | (50%, n = 4)** |
| Rat β-CGRP | 37 | 5.16 × 10⁵ | 7.85 × 10⁻⁵ | 0.152 |

*Affinities for α-CGRPs (rat and human) were determined in a high-resolution two-part experiment, in which the dissociation phase was monitored for 2 hours (the values for kₒₙ, k_{off}, and K_{D} represent the average of n replicate experiments with the standard deviation expressed as a percent variance). Affinities for β-CGRPs (rat and human) were determined by global analysis using only a 20-min dissociation phase, which was not accurate enough to quantify their extremely offrates (their offrates are likely slower than stated here and therefore their affinities are likely even higher). Antibody G1 Fab dissociated extremely slowly from all CGRPs (except α-rat CGRP) with offrates that approached the resolution limit of the Biacore assay (especially at 25° C.).
**Solution affinity determined by measuring the depletion of binding responses detected at CGRP on the chip for antibody G1 Fab pre-incubated with solution-based rat α-CGRP competitor.

Table 6 below shows antibodies having the amino acid sequence variation as compared to antibody G1 and their affinities to both rat α-CGRP and human α-CGRP. All amino acid substitutions of the variants shown in Table 6 are described relative to the sequence of G1. The binding affinities of Fab fragments were determined by Biacore by flowing them across CGRPs on a SA chip.

TABLE 6
Amino acid sequences and binding affinity data for antibody G1 variants
determined at 37° C. by Biacore.

| | | | | | α-rat | α-rat | α-human | α-human |
|---|---|---|---|---|---|---|---|---|
| Clone | L1 | L2 | H2 | HC-FW3 | k_{off} (1/s) | K_{D} (nM) | k_{off} (1/s) | K_{D} (nM) |
| G1 | | | | | 3.99 × 10⁻⁴ | 2.57 | 3.63 × 10⁻⁵ | 0.063 |
| M1 | | | | A100L | 1.10 × 10⁻³ | | 1.73 × 10⁻⁴ | |
| M2 | | | | L99A | 2.6 × 10⁻³ | 58 | 3.1 × 10⁻⁴ | 3 |
| | | | | A100R | | | | |
| M3 | | | | L99A | 2.0 × 10⁻³ | 61 | 2.1 × 10⁻⁴ | 1.7 |
| | | | | A100S | | | | |
| M4 | | | | L99A | 1.52 × 10⁻³ | 84.4 | 6.95 × 10⁻⁵ | 0.43 |
| | | | | A100V | | | | |
| M5 | | | | L99A | 7.35 × 10⁻⁴ | 40.8 | 3.22 × 10⁻⁵ | 0.20 |
| | | | | A100Y | | | | |
| M6 | | | | L99N | 7.84 × 10⁻⁴ | 43.6 | 1.33 × 10⁻⁴ | 0.83 |
| M7 | | | | L99N | 9.18 × 10⁻⁴ | 51.0 | 2.43 × 10⁻⁴ | 1.52 |
| | | | | A100C | | | | |
| M8 | | | | L99N | 7.45 × 10⁻⁴ | 41.4 | 9.20 × 10⁻⁵ | 0.58 |
| | | | | A100G | | | | |
| M9 | | | | L99N | n.d. | n.d. | 1.00 × 10⁻⁵ | 0.06 |
| | | | | A100Y | | | | |
| M10 | | | | L99S | 1.51 × 10⁻³ | 83.9 | 1.73 × 10⁻⁴ | 1.08 |
| | | | | A100S | | | | |
| M11 | | | | L99S | 4.83 × 10⁻³ | 268.3 | 2.83 × 10⁻⁴ | 1.77 |
| | | | | A100T | | | | |
| M12 | | | | L99S | 1.94 × 10⁻³ | 107.8 | 1.01 × 10⁻⁴ | 0.63 |
| | | | | A100V | | | | |
| M13 | | | | L99T | 1.84 × 10⁻³ | 102.2 | 1.86 × 10⁻⁴ | 1.16 |
| | | | | A100G | | | | |
| M14 | | | | L99T | n.d. | n.d. | 1.00 × 10⁻⁵ | 0.06 |
| | | | | A100K | | | | |
| M15 | | | | L99T | 1.15 × 10⁻³ | 63.9 | 1.58 × 10⁻⁵ | 0.10 |
| | | | | A100P | | | | |
| M16 | | | | L99T | 9.96 × 10⁻⁴ | 55.3 | 1.65 × 10⁻⁴ | 1.03 |
| | | | | A100S | | | | |
| M17 | | | | L99T | 2.06 × 10⁻³ | 114.4 | 1.85 × 10⁻⁴ | 1.16 |
| | | | | A100V | | | | |
| M18 | | | | L99V | 1.22 × 10⁻³ | 67.8 | 7.03 × 10⁻⁵ | 0.44 |
| | | | | A100G | | | | |
| M19 | | | | L99V | n.d. | n.d. | 1.00 × 10⁻⁵ | 0.06 |
| | | | | A100R | | | | |
| M20 | R28W | | | L99R | 1.44 × 10⁻³ | 80.0 | 1.36 × 10⁻⁴ | 0.85 |
| | | | | A100L | | | | |
| M21 | R28W | | | L99S | 6.95 × 10⁻⁴ | 15.2 | 1.42 × 10⁻⁴ | 1.23 |
| M22 | R28W | | | L99T | 1.10 × 10⁻³ | 61.1 | 1.16 × 10⁻⁴ | 0.73 |
| M23 | R28G | | | L99T | 7.99 × 10⁻⁴ | 44.4 | 1.30 × 10⁻⁴ | 0.81 |
| | | | | A100V | | | | |
| M24 | R28L | | | L99T | 1.04 × 10⁻³ | 57.8 | 1.48 × 10⁻⁴ | 0.93 |
| | | | | A100V | | | | |
| M25 | R28N | | | L99T | 1.4 × 10⁻³ | 76 | 1.4 × 10⁻⁴ | 1.3 |
| | | | | A100V | | | | |
| M26 | R28N | | A57G | L99T | 9.24 × 10⁻⁴ | 51.3 | 1.48 × 10⁻⁴ | 0.93 |
| | | | | A100V | | | | |
| M27 | R28N | | | L99T | 3.41 × 10⁻³ | 189.4 | 3.57 × 10⁻⁴ | 2.23 |
| | T30A | | | A100V | | | | |
| M28 | R28N | | E54R | L99T | 1.25 × 10⁻³ | 69.4 | 9.96 × 10⁻⁵ | 0.62 |
| | T30D | | A57N | A100V | | | | |
| M29 | R28N | | | L99T | 3.59 × 10⁻³ | 199.4 | 3.80 × 10⁻⁴ | 2.38 |
| | T30G | | | A100V | | | | |
| M30 | R28N | | E54K | L99T | 6.38 × 10⁻³ | 354.4 | 5.90 × 10⁻⁴ | 3.69 |
| | T30G | | A57E | A100V | | | | |
| M31 | R28N | | E54K | L99T | 3.61 × 10⁻³ | 200.6 | 3.47 × 10⁻⁴ | 2.17 |
| | T30G | | A57G | A100V | | | | |
| M32 | R28N | | E54K | L99T | 2.96 × 10⁻³ | 164.4 | 2.71 × 10⁻⁴ | 1.69 |
| | T30G | | A57H | A100V | | | | |
| M33 | R28N | | E54K | L99T | 9.22 × 10⁻³ | 512.2 | 7.50 × 10⁻⁴ | 4.69 |
| | T30G | | A57N | A100V | | | | |
| | | | S58G | | | | | |
| M34 | R28N | | E54K | L99T | 2.17 × 10⁻³ | 120.6 | 6.46 × 10⁻⁴ | 4.04 |
| | T30G | | A57N | A100V | | | | |
| | | | S58T | | | | | |
| M35 | R28N | | E54K | L99T | 3.99 × 10⁻³ | 221.7 | 3.39 × 10⁻⁴ | 2.12 |
| | T30G | | A57S | A100V | | | | |
| M36 | R28N | | | L99T | 4.79 × 10⁻³ | 266.1 | 2.39 × 10⁻⁴ | 1.49 |
| | T30R | | | A100V | | | | |
| M37 | R28N | | A57G | L99T | 1.45 × 10⁻³ | 80.6 | 2.26 × 10⁻⁴ | 1.41 |
| | T30S | | | A100V | | | | |
| M38 | R28N | | | L99T | 5.11 × 10⁻³ | 283.9 | 2.18 × 10⁻⁴ | 1.36 |
| | T30W | | | A100V | | | | |
| M39 | R28N | G50A | A57N | L99T | 9.95 × 10⁻³ | 552.8 | 4.25 × 10⁻⁴ | 2.66 |
| | | L56T | S58Y | A100V | | | | |
| M40 | R28N | G50A | E54K | L99T | 0.36 | 20000.0 | 1.28 × 10⁻³ | 8.00 |
| | | L56T | A57L | A100V | | | | |
| M41 | R28N | G50A | E54K | L99T | 4.53 × 10⁻³ | 251.7 | 2.10 × 10⁻⁴ | 1.31 |
| | | L56T | A57N | A100V | | | | |
| | | | E64D | | | | | |
| M42 | R28N | G50A | E54K | L99T | 7.52 × 10⁻³ | 417.8 | 4.17 × 10⁻⁴ | 2.61 |
| | | L56T | A57N | A100V | | | | |
| | | | H61F | | | | | |
| M43 | R28N | G50A | E54K | L99T | 4.53 × 10⁻³ | 251.7 | 2.63 × 10⁻⁴ | 1.64 |
| | | L56T | A57N | A100V | | | | |
| | | | S58C | | | | | |
| M44 | R28N | G50A | E54K | L99T | 6.13 × 10⁻³ | 443 | 2.10 × 10⁻⁴ | 2.05 |
| | | L56T | A57N | A100V | | | | |
| | | | S58E | | | | | |
| M45 | R28N | G50A | E54K | L99T | 5.58 × 10⁻³ | 259 | 2.11 × 10⁻⁴ | 1.85 |
| | | L56T | A57N | A100V | | | | |
| | | | S58E | | | | | |
| | | | E64D | | | | | |
| M46 | R28N | G50A | E54K | L99T | 2.94 × 10⁻³ | 163.3 | 5.39 × 10⁻⁴ | 3.37 |
| | | L56T | A57N | A100V | | | | |
| | | | S58E | | | | | |
| | | | H61F | | | | | |
| M47 | R28N | G50A | E54K | L99T | 8.23 × 10⁻³ | 457.2 | 3.32 × 10⁻⁴ | 2.08 |
| | | L56T | A57N | A100V | | | | |
| | | | S58G | | | | | |
| M48 | R28N | G50A | E54K | L99T | 0.0343 | 1905.6 | 8.42 × 10⁻⁴ | 5.26 |
| | | L56T | A57N | A100V | | | | |
| | | | S58L | | | | | |
| M49 | R28N | G50A | E54K | L99T | 0.0148 | 822.2 | 5.95 × 10⁻⁴ | 3.72 |
| | | L56T | A57N | A100V | | | | |
| | | | S58Y | | | | | |
| | | | H61F | | | | | |
| M50 | R28N | G50A | E54K | L99T | 5.30 × 10⁻³ | 294.4 | 4.06 × 10⁻⁴ | 2.54 |
| | | L56T | A57R | A100V | | | | |
| M51 | R28N | L56I | E54K | L99T | 1.18 × 10⁻³ | 65.6 | 1.31 × 10⁻⁴ | 0.82 |
| | | | A57G | A100V | | | | |
| M52 | R28N | L56I | E54K | L99T | 2.29 × 10⁻³ | 127.2 | 2.81 × 10⁻⁴ | 1.76 |
| | | | A57N | A100V | | | | |
| | | | S58A | | | | | |
| M53 | R28N | L56I | E54K | L99T | 1.91 × 10⁻³ | 106.1 | 3.74 × 10⁻⁴ | 2.34 |
| | | | A57N | A100V | | | | |
| | | | S58G | | | | | |
| M54 | R28N | G50A | E54K | L99T | 2.16 × 10⁻³ | 120.0 | 1.79 × 10⁻³ | 11.19 |
| | T30A | | A57N | A100V | | | | |
| | | | S58P | | | | | |
| M55 | R28N | L56S | E54K | L99T | 5.85 × 10⁻³ | 325.0 | 4.78 × 10⁻⁴ | 2.99 |
| | T30A | | A57N | A100V | | | | |
| | | | S58E | | | | | |
| | | | E64D | | | | | |
| M56 | R28N | L56S | E54K | L99T | 9.35 × 10⁻³ | 519.4 | 4.79 × 10⁻⁴ | 2.99 |
| | T30D | | A57N | A100V | | | | |
| | | | H61F | | | | | |
| M57 | R28N | L56S | E54K | L99T | 0.0104 | 1,200 | 3.22 × 10⁻⁴ | 3.08 |
| | T30D | | A57N | A100V | | | | |
| | | | S58E | | | | | |
| M58 | R28N | L56S | E54K | L99T | No binding | n.d. | 1.95 × 10⁻³ | 12.19 |
| | T30D | | A57N | A100V | | | | |
| | | | S58I | | | | | |
| | | | H61F | | | | | |
| M59 | R28N | L56S | E54K | L99T | 0.0123 | 683.3 | 5.24 × 10⁻⁴ | 3.28 |
| | T30D | | A57N | A100V | | | | |
| | | | S58N | | | | | |
| | | | H61F | | | | | |
| M60 | R28N | L56S | E54K | L99T | 0.0272 | 1511.1 | 9.11 × 10⁻⁴ | 5.69 |
| | T30D | | A57N | A100V | | | | |
| | | | S58R | | | | | |
| | | | H61F | | | | | |
| M61 | R28N | A51H | E54Q | L99T | 5.21 × 10⁻³ | 289.4 | 4.59 × 10⁻⁴ | 2.87 |
| | T30G | | A57N | A100V | | | | |
| | | | H61F | | | | | |
| M62 | R28N | A51H | E54K | L99T | 5.75 × 10⁻³ | 242 | 5.57 × 10⁻⁴ | 5.86 |
| | T30G | L56T | A57N | A100V | | | | |
| | | | S58E | | | | | |
| M63 | R28N | G50A | E54K | L99T | 2.65 × 10⁻³ | 147.2 | 1.50 × 10⁻³ | 9.38 |
| | T30G | | A57N | A100V | | | | |
| | | | S58T | | | | | |
| M64 | R28N | G50A | E54K | L99T | 0.0234 | 1300.0 | 1.32 × 10⁻³ | 8.25 |
| | T30G | | A57N | A100V | | | | |
| | | | S58V | | | | | |
| M65 | R28N | G50A | E54K | L99T | 4.07 × 10⁻³ | 226.1 | 8.03 × 10⁻⁴ | 5.02 |
| | T30G | L56I | A57C | A100V | | | | |
| M66 | R28N | L56I | E54K | L99T | 5.11 × 10⁻³ | 283.9 | 5.20 × 10⁻⁴ | 3.25 |
| | T30G | | A57E | A100V | | | | |
| M67 | R28N | L56I | E54K | L99T | 1.71 × 10⁻³ | 95.0 | 8.20 × 10⁻⁴ | 5.13 |
| | T30G | | A57F | A100V | | | | |
| M68 | R28N | L56I | E54K | L99T | 6.76 × 10⁻³ | 375.6 | 4.28 × 10⁻⁴ | 2.68 |
| | T30G | | A57N | A100V | | | | |
| | | | S58D | | | | | |
| | | | E64D | | | | | |
| M69 | R28N | L56I | E54K | L99T | 1.81 × 10⁻³ | 100.6 | 7.33 × 10⁻⁴ | 4.58 |
| | T30G | | A57N | A100V | | | | |
| | | | S58E | | | | | |
| M70 | R28N | L56I | E54K | L99T | 6.07 × 10⁻³ | 337.2 | 5.59 × 10⁻⁴ | 3.49 |
| | T30G | | A57S | A100V | | | | |
| M71 | R28N | L56I | E54K | L99T | 2.12 × 10⁻³ | 117.8 | 1.28 × 10⁻³ | 8.00 |
| | T30G | | A57Y | A100V | | | | |
| M72 | R28N | L56S | E54K | L99T | 3.95 × 10⁻³ | 219.4 | 4.00 × 10⁻⁴ | 2.50 |
| | T30G | | | A100V | | | | |
| M73 | R28N | L56S | E54K | L99T | 3.00 × 10⁻³ | 166.7 | 2.55 × 10⁻⁴ | 1.59 |
| | T30G | | A57N | A100V | | | | |
| | | | S58Y | | | | | |
| | | | E64D | | | | | |
| M74 | R28N | L56S | E54K | L99T | 6.03 × 10⁻³ | 335.0 | 5.97 × 10⁻⁴ | 3.73 |
| | T30G | | A57S | A100V | | | | |
| M75 | R28N | L56S | E54K | L99T | 1.87 × 10⁻² | 1038.9 | 1.16 × 10⁻³ | 7.25 |
| | T30G | | A57V | A100V | | | | |
| M76 | R28N | G50A | A57G | L99T | 1.16 × 10⁻³ | 64.4 | 3.64 × 10⁻⁴ | 2.28 |
| | T30S | L56T | | A100V | | | | |
| M77 | R28N | G50A | E54K | L99T | 0.0143 | 794.4 | 4.77 × 10⁻⁴ | 2.98 |
| | T30S | L56T | A57D | A100V | | | | |
| M78 | R28N | G50A | E54K | L99T | 0.167 | 9277.8 | 1.31 × 10⁻³ | 8.19 |
| | T30S | L56T | A57N | A100V | | | | |
| | | | S58T | | | | | |
| M79 | R28N | G50A | E54K | L99T | 0.19 | 10555.6 | 1.29 × 10⁻³ | 8.06 |
| | T30S | L56T | A57P | A100V | | | | |
| M80 | R28N | L56I | E54K | L99T | 0.0993 | 5516.7 | 2.09 × 10⁻³ | 13.06 |
| | T30S | | A57N | A100V | | | | |
| | | | S58V | | | | | |
| M81 | R28N | L56S | E54K | L99T | 4.29 × 10⁻³ | 238.3 | 4.90 × 10⁻⁴ | 3.06 |
| | T30S | | A57N | A100V | | | | |
| | | | S58E | | | | | |
| M82 | R28N | A51H | A57N | L99T | 6.99 × 10⁻³ | 388.3 | 8.77 × 10⁻⁴ | 5.48 |
| | T30V | L56T | | A100V | | | | |
| M83 | R28N | A51H | E54K | L99T | No binding | n.d. | 9.33 × 10⁻⁴ | 5.83 |
| | T30V | L56T | A57N | A100V | | | | |
| | | | S58M | | | | | |
| | | | H61F | | | | | |
| M84 | R28N | A51H | E54N | L99T | 1.76 × 10⁻² | 977.8 | 1.08 × 10⁻³ | 6.75 |
| | T30V | L56T | A57N | A100V | | | | |

All CDRs including both Kabat and Chothia CDRs. Amino acid residues are numbered sequentially (see FIG. 5). All clones have L3 + H1 + H3 sequences identical to G1.
K_{D} = k_{off}/kₒₙ. All k_{off} values were determined in a screening mode except those that are underlined, which were obtained by global analysis of a Fab concentration series (G1 was analyzed in a high-resolution mode). Underlined K_{D} values were therefore determined experimentally by measuring kₒₙ. Other kₒₙ values were estimated to be the same as M25.
n.d. = not determined

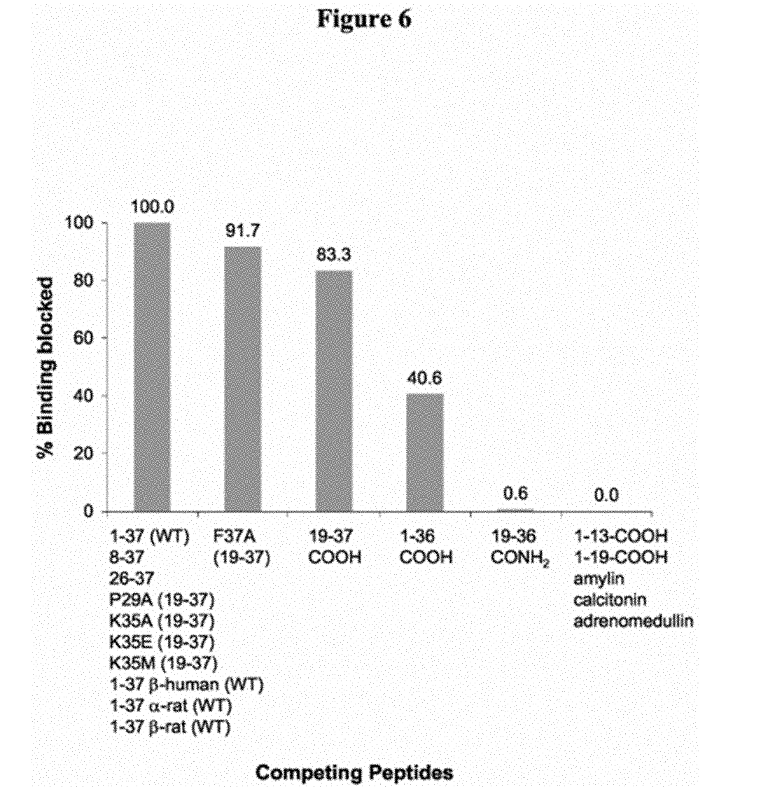

To determine the epitope on human α-CGRP that is recognized by antibody G1, Biacore assays described above were used. Human α-CGRP was purchased as an N-biotinylated version to enable its high-affinity capture via SA sensor chips. The binding of G1 Fab fragment to the human α-CGRP on the chip in the absence or presence of a CGRP peptide was determined. Typically, a 2000:1 mol peptide/Fab solution (e.g., 10 μM peptide in 50 nM G1 Fab) was injected across human α-CGRP on the chip. FIG. 6 shows the percentage of binding blocked by competing peptide. Data shown in FIG. 6 indicate that peptides that block 100% binding of G1 Fab to human α-CGRP are 1-37 (WT), 8-37, 26-37, P29A (19-37), K35A (19-37), K35E (19-37), and K35M (19-37) of human α-CGRP; 1-37 of β-CGRP (WT); 1-37 of rat α-CGRP (WT); and 1-37 of rat β-CGRP (WT). All these peptides are amidated at the C-terminus. Peptides F37A (19-37) and 19-37 (the latter not amidated at the C-terminus) of human α-CGRP also blocked about 80% to 90% of binding of G1 Fab to human α-CGRP. Peptide 1-36 (not amidated at the C-terminus) of human α-CGRP blocked about 40% of binding of G1 Fab to human α-CGRP. Peptide fragment 19-36 (amidated at the C-terminus) of human α-CGRP; peptide fragments 1-13 and 1-19 of human α-CGRP (neither of which are amidated at the C-terminus); and human amylin, calcitonin, and adrenomedullin (all amidated at the C-terminus) did not compete with binding of G1 Fab to human α-CGRP on the chip. These data demonstrate that G1 targets a C-terminal epitope of CGRP and that both the identity of the most terminal residue (F37) and its amidation is important for binding.

Binding affinities of G1 Fab to variants of human α-CGRP (at 37° C.) was also determined. Table 7 below shows the affinities as measured directly by titrating G1 Fab across N-biotinylated human α-CGRP and variants on the chip. Data in Table 7 indicate that antibody G1 binds to a C-terminal epitope with F37 and G33 being the most important residues. G1 does not bind to CGRP when an extra amino acid residue (alanine) is added at the C-terminal (which is amidated).

TABLE 7
Binding affinities of G1 Fab to human α-CGRP and variants
measured at 37° C. (see Table 4 for their amino acid sequences)

| CGRP on chip | kₒₙ (1/Ms) | k_{off} (1/s) | K_{D} (nM) |
|---|---|---|---|
| 1-37 (WT) | 4.68 × 10⁵ | 7.63 × 10⁻⁵ | 0.16 (high resolution |
| | | | K_{D} = 0.06) |
| 19-37 | 4.60 × 10⁵ | 7.30 × 10⁻⁵ | 0.16 |
| 25-37 | 3.10 × 10⁵ | 8.80 × 10⁻⁵ | 0.28 |
| F27A (25-37) | 3.25 × 10⁵ | 1.24 × 10⁻⁴ | 0.38 |
| V28A (25-37) | 3.32 × 10⁵ | 9.38 × 10⁻⁵ | 0.28 |
| P29A (25-37) | 2.26 × 10⁵ | 1.78 × 10⁻⁴ | 0.79 |
| T30A (25-37) | 1.79 × 10⁵ | 8.41 × 10⁻⁵ | 0.47 |
| N31A (25-37) | 2.17 × 10⁵ | 1.14 × 10⁻⁴ | 0.53 |
| V32A (25-37) | 2.02 × 10⁵ | 3.46 × 10⁻⁴ | 1.71 |
| G33A (25-37) | 2.07 × 10⁵ | 0.0291 | 141 |
| S34A (25-37) | 2.51 × 10⁵ | 7.64 × 10⁻⁴ | 3.04 |
| K35A (19-37) | 2.23 × 10⁵ | 2.97 × 10⁻⁴ | 1.33 |
| K35E (19-37) | 5.95 × 10⁴ | 5.79 × 10⁻⁴ | 9.73 |
| K35M (19-37) | 2.63 × 10⁵ | 1.34 × 10⁻⁴ | 0.51 |
| K35Q (19-37) | 1.95 × 10⁵ | 2.70 × 10⁻⁴ | 1.38 |
| F37A (25-37) | 8.90 × 10⁴ | 8.48 × 10⁻³ | 95 (solution K_{D} = 172 nM) |
| 38A (25-38A) | — | — | No binding detected |

The above data indicate that the epitope that antibody G1 binds is on the C-terminal end of human α-CGRP, and amino acids 33 and 37 on human α-CGRP are important for binding of antibody G1. Also, the amidation of residue F37 is important for binding.

### Example 5: CH Clinical Study Protocol

### General Design

This is a 68-week extension study to evaluate the long term safety of TEV-48125 in adult patients with CH. Eligible patients with ECH and CCH will receive TEV-48125 during this study as summarized in table below.

### Summary of Treatments During Study

| | Treatment group | |
|---|---|---|
| Study number | in pivotal study | Treatment in the long-term safety extension study^{a),b)} |
| Study | TEV-48125 900-mg | TEV-48125 at 225 mg sc monthly (approximately every |
| TV48125- | iv loading dose | 4 weeks) through week 36 |
| CNS-30056 | group^{c)} | |
| (ECH) | TEV-48125 675-mg | TEV-48125 at 675 mg sc quarterly (approximately every |
| | sc quarterly group^{d)} | 12 weeks) through week 36 |
| | Placebo group^{e)} | TEV-48125 at 675 mg sc quarterly (approximately every |
| | | 12 weeks) through week 36 |
| Study | TEV-48125 900-mg | TEV-48125 at 225 mg sc monthly (approximately every |
| TV48125- | iv loading dose | 4 weeks) through week 36 |
| CNS-30057 | group^{c} | |
| (CCH) | TEV-48125 675-mg | TEV-48125 at 225 mg sc monthly (approximately every |
| | sc loading dose | 4 weeks) through week 36 |
| | group^{f)} | |
| | Placebo group^{e} | TEV-48125 675-mg sc loading dose followed by monthly |
| | | (approximately every 4 weeks) TEV-48125 at 225 mg sc |
| | | through week 36 |

^{a)}In order to maintain blinding throughout the study, the number of sc injections at each visit will be the same for all patients rolling over from Study TV48125-CNS-30056, regardless of their assigned treatment group. Thus, patients will receive 3 sc injections of either test IMP (1.5 mL-injections each containing TEV-48125 at a concentration of 150 mg/mL) or placebo IMP (1.5-mL injections) at visits 1, 4, 7, and 10, and a single sc injection of test IMP or placebo IMP at visits 2, 3, 5, 6, 8, and 9.
^{b)}In order to maintain blinding throughout the study, the number of sc injections at each visit will be the same for all patients rolling over from Study TV48125-CNS-30057, regardless of their assigned treatment group. Thus, patients will receive 3 sc injections of either test IMP (1.5 mL-injections each containing TEV-48125 at a concentration of 150 mg/mL) or 1 sc injection of test IMP and 2 sc injections of placebo IMP (1.5-mL injections) at visit 1.
^{c)}TEV-48125 at 900 mg iv at visit 2 (week 0) of the pivotal study and TEV-48125 at 225 mg sc at visits 3 and 4 (weeks 4 and 8, respectively) of the pivotal study.
^{d)}TEV-48125 at 675 mg sc at visit 2 (week 0) of the pivotal study and placebo sc at visits 3 and 4 (weeks 4 and 8, respectively) of the pivotal study.
^{e)}Placebo iv and sc at visit 2 (week 0) of the pivotal study and placebo sc at visits 3 and 4 (weeks 4 and 8, respectively) of the pivotal study.
^{f)}TEV-48125 at 675 mg sc at visit 2 (week 0) of the pivotal study and TEV-48125 at 225 mg sc at visits 3 and 4 (weeks 4 and 8, respectively) of the pivotal study.
CCH = chronic cluster headache;
ECH = episodic cluster headache;
IMP = investigational medicinal product;
iv = intravenous;
sc = subcutaneous.

Patients with a diagnosis of ECH who experience CH remission, defined as no CH attacks for 12 successive weeks at any time after starting IMP (i.e., administration of the first dose of IMP in the pivotal study), will stop treatment. If CH attacks resume within 12 weeks after stopping treatment, patients will restart treatment at their previous dose regimen through week 36. If CH attacks resume after more than 12 weeks after stopping treatment, patients will restart TEV-48125 treatment at 675 mg sc quarterly through week 36.

Patients with a diagnosis of CCH who experience CH remission, defined as no CH attacks for 24 successive weeks at any time after starting IMP (i.e., administration of the first dose of IMP in the pivotal study), will stop treatment. If CH attacks resume within 12 weeks after stopping treatment, patients will restart TEV-48125 treatment at 225 mg sc monthly through week 36. If CH attacks resume after more than 12 weeks after stopping treatment, patients will restart TEV-48125 treatment with a 675-mg sc loading dose followed by 225-mg sc monthly through week 36.

All patients will return to the investigational center approximately every 4 weeks after administration of the first dose of the IMP (visit 1 [week 0]) through the end-of-treatment (EOT) visit (visit 11 [week 40]), which will occur approximately 4 weeks after administration of the final dose of the IMP (week 36). Patients will return for a follow-up visit (visit 12) to evaluate ADAs, TEV-48125 concentrations, biomarkers, and safety (adverse events and concomitant medications) approximately 7.5 months after the last dose of the IMP. This visit is for the purpose of evaluating ADAs, TEV-48125 concentrations, biomarkers, and safety only. Patients who withdraw from the study before completing the 40 week treatment period will have EOT visit procedures and assessments performed on the last day the patients received the IMP or as soon as possible thereafter, and they will be asked to return for a follow-up visit approximately 7.5 weeks after their last dose of IMP.

CH attack information will be captured daily throughout the treatment period (i.e., visit 1 [week 0] through visit 11 [week 40]) using an electronic diary device. Assessments of CH-related disability, change in quality of life, and health status (using the Hospital Anxiety and Depression Scale [HADS], EuroQol 5 Dimension [EQ-5D] questionnaire, 12-Item Short-Form Health Survey [SF 12], Impact on Partner and Family questionnaire, and Work Productivity and Activity Impairment [WPAI] questionnaire); satisfaction with treatment (using the Patient Perceived Satisfactory Improvement [PPSI] and the Patient Global Impression of Change [PGIC] scale); safety evaluations; blood collection for pharmacokinetic, immunogenicity, and biomarker analyses; and urine sampling for biomarker analysis will be performed at prespecified time points.

The long-term safety of TEV-48125 in patients with CH will be evaluated through adverse event and concomitant medication inquiries, ECGs, vital signs measurements, clinical laboratory tests, physical examinations, injection site assessments, assessments for anaphylaxis and hypersensitivity, and administration of the eC-SSRS. The end of study is defined as the date the last patient attends the follow-up visit.

Investigational medicinal product is defined as the test IMP and placebo IMP. Details of the test and placebo IMPs are presented in the table below.

Investigational Medicinal Products Used in the Study

| IMP name | Test IMP | Placebo IMP |
|---|---|---|
| Trade name and | TEV-48125 (formerly LBR-101, PF- | n/a |
| INN, if applicable, | 04427429, or RN 307) | |
| or | | |
| company-assigned | | |
| number | | |
| Formulation | solution for injection | solution for injection |
| Unit dose | 225 mg/1.5 mL | n/a |
| strength(s) | 675 mg sc quarterly, 225 mg sc | |
| dosage level(s) | monthly, or 675 mg sc loading dose | |
| | followed by 225 mg monthly | |
| Route of | TEV-48125 will be administered as sc | Placebo will be administered as sc |
| administration | injections (1.5 mL per injection) by | injections (1.5 mL per injection) by |
| | qualified study personnel at the | qualified study personnel at the |
| | investigational center. | investigational center. |
| Packaging | TEV-48125 will be provided in | Placebo will be provided in prefilled |
| | prefilled syringes contained in uniquely | syringes contained in uniquely |
| | numbered kits and stored (refrigerated | numbered kits and stored (refrigerated |
| | at 2° C. to 8° C.) at the investigational | at 2° C. to 8° C.) at the investigational |
| | centers. Prefilled syringes (1.5 mL) | centers. Prefilled syringes (1.5 mL) |
| | will contain TEV-48125 at a | will contain the same vehicle and |
| | concentration of 150 mg/mL. | excipients as those for active infusion |
| | | and injection. |

IMP = investigational medicinal product;
INN = International Nonproprietary Names;
n/a = not applicable;
sc = subcutaneous.

The recommended sc injection sites follow the National Institutes of Health Patient Education Guidelines of September 2015, which are available at the following website: cc.nih.gov/ccc/patient_education/pepubs/subq.pdf. The suggested sites of injection are back of upper arms, lower abdomen/belly/waistline, and front of thighs. At each visit, the injections should be given in a different location (eg, not in precisely the same place), and study staff member(s) responsible for administration of injections should inspect previous injection sites to ensure that they are free of bruising and tenderness and that proper rotation of sites is performed. The total number of sc injections and their locations will be recorded for each dosing visit (visits 1 through 10). A 1.5-mL volume from each prefilled syringe must be injected sc for dosing to be considered complete.

### Test Investigational Medicinal Product

TEV-48125 is a humanized IgG2a/kappa monoclonal antibody derived from a murine precursor.

### Starting Dose and Dose Levels

Patients rolling over from Study TV48125-CNS-30056 with ECH:
Patients who were in the TEV-48125 900-mg iv loading dose group will receive TEV-48125 at 225 mg sc as a single injection at visit 1 and every 4 weeks thereafter through week 36 (visit 10). For blinding, these patients will also receive 2 placebo sc injections at visits 1, 4, 7, and 10.
Patients who were in the placebo group and the TEV-48125 675 mg sc quarterly group will receive TEV-48125 at 675 mg sc as three injections (225 mg/1.5 mL) at visit 1 and every 12 weeks thereafter through week 36 (visit 10). For blinding, patients will receive 1 single placebo sc injections at visits 2, 3, 5, 6, 8, and 9.

Patients with a diagnosis of ECH who experience CH remission, defined as no CH attacks for 12 successive weeks at any time after starting IMP (i.e., administration of the first dose of IMP in the pivotal study), will stop treatment. If CH attacks resume within 12 weeks after stopping treatment, patients will restart treatment at their previous dose regimen through week 36. If CH attacks resume after more than 12 weeks after stopping treatment, patients will restart TEV-48125 treatment at 675 mg sc quarterly through week 36.

Patients rolling over from Study TV48125-CNS-30057 with CCH:
Patients who were in the TEV-48125 900-mg iv loading dose group and the TEV-48125 675-mg sc loading dose group will receive TEV-48125 at 225 mg as a single sc injection (225 mg/1.5 mL) at visit 1 and every 4 weeks thereafter through week 36 (visit 10). For blinding, these patients will also receive 2 sc placebo injections at visit 1.
Patients who were in the placebo group will receive a loading dose of TEV-48125 at 675 mg as 3 sc injections (225 mg/1.5 mL) at visit 1 and TEV-48125 at 225 mg administered as a single sc injection every 4 weeks thereafter through week 36 (visit 10).

Patients with a diagnosis of CCH who experience CH remission, defined as no CH attacks for 24 successive weeks at any time after starting IMP (i.e., administration of the first dose of IMP in the pivotal study), will stop treatment. If CH attacks resume within 12 weeks after stopping treatment, patients will restart TEV-48125 treatment at 225 mg sc monthly through week 36. If CH attacks resume after more than 12 weeks after stopping treatment, patients will restart TEV-48125 treatment with a 675-mg sc loading dose followed by 225-mg sc monthly through week 36.

### Example 6: CCH Clinical Study Protocol

A Multicenter, Randomized, Double-Blind, Double-Dummy, Placebo Controlled, Parallel Group Study Comparing the Efficacy and Safety of Two Dose Regimens (Intravenous/Subcutaneous and Subcutaneous) of TEV-48125 versus Placebo for the Prevention of Chronic Cluster Headache. This is a 16-week, multicenter, randomized, double-blind, double-dummy, placebo-controlled, parallel group study to compare the efficacy and safety of 2 dose regimens of TEV-48125 versus placebo in adult patients for the prevention of CCH. The study will consist of a screening visit, a run in period lasting approximately 4 weeks (+3 days), and a 12 week double blind treatment period. Patients will complete a screening visit (visit 1) after providing written informed consent, and eligible patients will enter a run-in period lasting at least 4 weeks (+3 days) during which they will enter baseline CH attack information into an electronic diary device daily. Patients will return to the study center after completing the run-in period (visit 2 [week 0]). Patients who have at least 10 CH attacks during the run-in period and who continue to meet eligibility criteria (including entry of CH attack information in an electronic diary demonstrating compliance for 85% of days during the run-in period) will be randomly assigned at visit 2 (week 0) in a 1:1:1 ratio to 1 of 3 treatment groups as follows:
1) TEV-48125 900-mg iv loading dose group: TEV-48125 at 900 mg administered via an approximately 1-hour iv infusion at visit 2 (week 0) followed by TEV-48125 at 225 mg administered as single sc injections (225 mg/1.5 mL) at visits 3 and 4 (weeks 4 and 8, respectively).
2) TEV-48125 675-mg sc loading dose group: TEV-48125 at 675 mg administered as 3 sc injections (225 mg/1.5 mL) at visit 2 (week 0) followed by TEV-48125 at 225 mg administered as single sc injections (225 mg/1.5 mL) at visits 3 and 4 (weeks 4 and 8, respectively).
3) Placebo group: placebo administered via an approximately 1-hour iv infusion and as 3 sc injections at visit 2 (week 0) followed by placebo administered as single sc injections at visits 3 and 4 (weeks 4 and 8, respectively).

In order to maintain blinding throughout the study, the number of infusions and injections at each visit will be the same for all patients regardless of the treatment group to which they are randomized. Thus, all patients will receive an iv infusion of the test IMP or placebo IMP followed by 3 sc injections of the test IMP or placebo IMP at visit 2 (week 0), and all patients will receive single sc injections of the test IMP or placebo IMP at visits 3 and 4 (weeks 4 and 8, respectively).

Randomization will be performed using electronic interactive response technology (IRT). Patients will be randomly assigned with stratification based on sex, country, and baseline concomitant preventive medications (yes/no) to the TEV-48125 900-mg iv loading dose group, the TEV-48125 675-mg sc loading dose group, or the placebo group in a 1:1:1 ratio.

Blinded treatment will be administered once monthly (i.e., approximately every 4 weeks) for a total of 3 months. Final study assessments will be performed at the final visit for this study (visit 5), approximately 12 weeks after administration of the first dose of the IMP. Upon satisfactory completion of the final study assessments, patients will be offered to enter a 68 week long-term safety study (Study TV48125-CNS-30058), consisting of a 40-week long-term treatment period and a final follow-up visit approximately 7.5 months after the last dose of the IMP. A separate protocol will be issued for the long-term safety study. Patients who do not enroll in the long-term safety study for any reason will be offered to enter the study for the purpose of evaluating ADAs, TEV-48125 concentrations, and safety (adverse events and concomitant medications) at approximately 7.5 months after receiving the last dose of the IMP.

CH attack information will be captured daily during the double-blind treatment period using an electronic diary device. Assessments of CH-related disability, change in quality of life, and health status (using the Hospital Anxiety and Depression Scale [HADS], EuroQol-5 Dimension [EQ-5D] questionnaire, 12-Item Short-Form Health Survey [SF-12], Impact on Partner and Family questionnaire, and Work Productivity and Activity Impairment [WPAI] questionnaire); satisfaction with treatment (using the PPSI and Patients' Global Impression of Change [PGIC] scale); safety evaluations (including eC-SSRS); blood collection for pharmacokinetics, immunogenicity, biomarker, and pharmacogenomics (unless not allowed per local regulation) analyses; and urine sampling for biomarker analysis will be performed at prespecified time points.

### Investigational Medicinal Products Used in the Study

Investigational medicinal product is defined as the test IMP and the placebo IMP. Details of the test and placebo IMPs are presented table below.

| IMP Name | Test IMP | Placebo IMP |
|---|---|---|
| Trade name and INN, if | TEV-48125 (formerly LBR-101, | n/a |
| applicable, or | PF-04427429, or RN307) | |
| company-assigned number | | |
| Formulation | Solution for injection | Solution for injection |
| Unit dose strength(s)/Dosage | 225 mg/1.5 mL | n/a |
| level(s) | 900 mg iv loading dose followed | |
| | by 225 mg sc monthly | |
| | or | |
| | 675 mg sc dose followed by | |
| | 225 mg sc monthly | |
| Route of Administration | TEV-48125 will be administered | Placebo will be administered as |
| | as iv infusions and sc injections | iv infusions and sc injections by |
| | by qualified study personnel at | qualified study personnel at the |
| | the study center. | study center. |
| Packaging | TEV-48125 will be provided in | Placebo will be provided in |
| | prefilled syringes contained in | prefilled syringes contained in |
| | uniquely numbered kits and | uniquely numbered kits and |
| | stored (refrigerated at 2° C. to | stored (refrigerated at 2° C. to |
| | 8° C.) on site. Prefilled syringes | 8° C.) on site. Prefilled syringes |
| | will contain TEV-48125 at a | will contain the same vehicle and |
| | concentration of 150 mg/mL. | excipients as those for active |
| | | infusion and injection. |

IMP = investigational medicinal product;
iv = intravenous;
n/a = not applicable;
sc = subcutaneous.

The TEV-48125 doses, regimens, and routes of administration to be evaluated in this double blind, double-dummy, placebo-controlled study were selected on the basis of three key factors. First, simulations suggest that Cₘₐₓ is the most significant pharmacokinetic parameter in the efficacy of TEV-48125 (in migraine). As CH is considered one of the most severe forms of pain a person can experience, treatments that provide quick and lasting relief (i.e., for the duration of the cluster period) are a priority for this patient population. Second, the biological nature of the disease mandates the need for any treatment to desensitize the third order neuron, not the second (as is the case in migraine), suggesting that high levels of blockade at the first neuron would be necessary. Third, the favorable safety profile of the drug and calculated safety margins based modelling and simulations, as well as clinical and nonclinical safety data on exposure, suggest that the proposed doses, regimens, and routes of administration will not present any safety concerns.

In the current study, high doses are planned for the first dose (900 mg iv or 675 mg sc) in order to provide a rapid response, especially following iv infusion where higher peak plasma concentrations (Cₘₐₓ) generally occur at or shortly after the end of infusion (median tmax values of 1.0 to 5.0 hours after starting the iv infusion) compared with 96 to 108 hours postdose for sc injections. The two forms of loading dose will provide data to confirm the benefit of either the iv or sc as loading dose. Monthly doses of TEV-48125 at 225 mg sc were added to both the initial dose of 900 mg iv and 675 mg sc for maintenance of efficacy. Based on modelling, the inclusion of a loading dose should allow patients to reach steady state faster. The monthly doses of 225 mg sc in this CCH population will account for the continuous attacks, with less than 1 month free of headaches between attacks, seen with this CH form.

Administration of IMP will be via the iv and sc routes. At visit 2, IMP (test or placebo) will be administered via an approximately 1-hour iv infusion followed by 3 sc injections of IMP (test or placebo). At visits 3 and 4, IMP (test or placebo) will be administered as single sc injections. The recommended sc injection sites follow the National Institutes of Health Patient Education Guidelines of September 2015, which are available at the following website: www.cc.nih.gov/ccc/patient_education/pepubs/subq.pdf. The suggested sites of injection are back of upper arms, lower abdomen/belly/waistline, and front of thighs. At each visit, the injections should be given in a different location (eg, not in precisely the same place), and study staff member(s) responsible for administration of injections should inspect previous injection sites to ensure that they are free of bruising and tenderness and that proper rotation of sites is performed. The total number of sc injections and their locations will be recorded for each dosing visit (visits 2 through 4, test or placebo). A 1.5-mL volume from each prefilled syringe must be injected sc for dosing to be considered complete.

### Test Investigational Medicinal Product

TEV-48125 is a humanized IgG2a/kappa monoclonal antibody derived from a murine precursor. TEV-48125 for CH is being developed for iv and sc administration.

Starting Dose and Dose Levels

Patients randomized to receive TEV-48125 will receive either 900 mg or 675 mg. At visit 2 (week 0), iv administration of treatment (IMP or placebo) will precede sc administration (IMP or placebo). Patients randomized to the 900-mg iv loading dose group will receive 900 mg of TEV-48125 as an approximately 1-hour iv infusion followed by 3 placebo sc injections at visit 2 (week 0) and TEV-48125 at 225 mg administered as single sc injections (225 mg/1.5 mL) at visits 3 and 4 (weeks 4 and 8, respectively). Patients in the 675-mg sc dose group will receive placebo as an approximately 1-hour iv infusion followed by TEV-48125 at 675 mg administered as 3 sc injections (225 mg/1.5 mL) at visit 2 (week 0) and TEV-48125 at 225 mg administered as single sc injections at visits 3 and 4 (weeks 4 and 8, respectively).

### Study Objectives and Endpoints

Primary Objective and Endpoint:

The primary objective of this study is to demonstrate the efficacy of fremanezumab in the prevention of chronic cluster headache (CCH) in adult patients.

The primary efficacy endpoint of this study is the mean change from baseline (run-in period) in the monthly average number of cluster headache (CH) attacks during the 12-week period after administration of the first dose of the investigational medicinal product (IMP), i.e., based on week 0 to 12 data.

Secondary Objective and Endpoints

A secondary objective of this study is to further demonstrate the efficacy of fremanezumab in the prevention of CCH in adult patients.

The secondary efficacy endpoints to further demonstrate efficacy are:
the proportion of patients with a ≥50% reduction from baseline (run-in period) in the monthly average number of CH attacks over the 12-week period after the administration of the first dose of the IMP, i.e., based on week 0 to 12 data;
the mean change from baseline (run-in period) in the number of CH attacks during the 4-week period after administration of the first dose of the IMP, i.e., based on week 0 to 4 data;
the mean change from baseline (run-in period) in the number of CH attacks during the 4-week period after administration of the third dose of the IMP, i.e., based on week 8 to 12 data;
the mean change from baseline (run-in period) in the weekly average number of days with use of cluster-specific acute headache medications (triptans and ergot compounds) during the 12-week period after administration of the first dose of the IMP, i.e., based on week 0 to 12 data;
the mean change from baseline (run-in period) in the weekly average number of days oxygen is used to treat CCH during the 12-week period after administration of the first dose of the IMP, i.e., based on week 0 to 12 data; and
assessment of patient's perceived improvement, as measured by the Patient-Perceived Satisfactory Improvement (PPSI) at 1, 4, 8, and 12 weeks after administration of the first dose of the IMP relative to baseline (day 0).

A secondary objective of this study is to evaluate the safety of fremanezumab in adult patients with CCH.

The secondary safety endpoints are as follows:
occurrence of adverse events throughout the study;
clinical laboratory (serum chemistry, hematology, coagulation, and urinalysis) test results at each visit;
vital signs (systolic and diastolic blood pressure, oral temperature, and pulse rate) measurements at each visit. Note: Oxygen saturation will be measured in cases of suspected anaphylaxis and severe hypersensitivity. Respiratory rate will also be measured in these cases but not as a standard vital sign;
12-lead electrocardiogram (ECG) findings at screening, baseline, and week 12
use of concomitant medication during the study;
clinically significant changes in physical examinations, including body weight;
injection site reaction (i.e., erythema, induration, and ecchymosis) and injection site pain assessments;
occurrence of hypersensitivity/anaphylaxis reactions; and
suicidal ideation and behavior as measured by the electronic Columbia Suicide Severity Rating Scale (eC-SSRS).

### Example 7: ECH Clinical Study Protocol

### General Design

This is a 13 week, multicenter, randomized, double-blind, double-dummy, placebo-controlled, parallel group study to compare the safety, and efficacy of two dose regimens of TEV-48125 versus placebo in adult patients for the prevention of ECH. The study will consist of a screening visit, a run in period lasting at least 1 week (+3 days), and a 12-week double blind treatment period.

Patients will complete a screening visit (visit 1) after providing written informed consent, and eligible patients will enter a run-in period lasting at least 1 week (+3 days) during which they will enter baseline CH attack information into an electronic diary device daily. Patients will return to the study center after completing the run-in period (visit 2 [week 0]). Patients who had at least 7 CH attacks during the run-in period and who continue to meet eligibility criteria (including entry of CH attack information in an electronic diary demonstrating compliance for 85% of days during the run-in period) will be randomly assigned at visit 2 (week 0) in a 1:1:1 ratio to 1 of 3 treatment groups as follows:
1) TEV-48125 900-mg iv loading dose group: TEV-48125 at 900 mg administered via an approximately 1-hour iv infusion at visit 2 (week 0) followed by TEV-48125 at 225 mg administered as single sc injections (225 mg/1.5 mL) at visits 3 and 4 (weeks 4 and 8, respectively)
2) TEV-48125 675-mg sc quarterly group: TEV-48125 at 675 mg administered as 3 sc injections (225 mg/1.5 mL) at visit 2 (week 0) followed by placebo administered as single sc injections at visits 3 and 4 (weeks 4 and 8, respectively)
3) Placebo group: placebo administered via an approximately 1-hour iv infusion and as 3 sc injections at visit 2 (week 0) followed by placebo administered as single sc injections at visits 3 and 4 (weeks 4 and 8, respectively)

In order to maintain blinding throughout the study, the number of infusions and injections at each visit will be the same for all patients regardless of the treatment group to which they are randomized. Thus, all patients will receive an iv infusion of test IMP or placebo IMP followed by 3 sc injections of test IMP or placebo IMP at visit 2 (week 0), and all patients will receive single sc injections of test IMP or placebo IMP at visits 3 and 4 (weeks 4 and 8, respectively).

Randomization will be performed using electronic interactive response technology (IRT). Patients will be randomly assigned with stratification based on sex, country, and baseline concomitant preventive medications (yes/no) to the TEV-48125 900-mg iv loading dose group, the TEV-48125 675 mg sc quarterly group, or the placebo group in a 1:1:1 ratio.

Blinded treatment will be administered once monthly (i.e., approximately every 4 weeks) for a total of 3 months. Final study assessments will be performed at the final visit for this study (visit 5), approximately 12 weeks after administration of the first dose of the IMP. Upon satisfactory completion of the final study assessments, patients will be offered to enter a 68 week long-term safety study (Study TV48125-CNS-30058), consisting of a 40-week long-term treatment period and a final follow-up visit approximately 7.5 months after the last dose of the IMP. A separate protocol will be issued for the long-term safety study. Patients who do not enroll in the long-term safety study for any reason will be offered to enter the study for the purpose of evaluating ADAs, TEV-48125 concentrations, and safety (adverse events and concomitant medications) at approximately 7.5 months after receiving the last dose of the IMP.

CH attack information will be captured daily during the double-blind treatment period using an electronic diary device. Assessments of CH-related disability, change in quality of life, and health status (using the Hospital Anxiety and Depression Scale [HADS], EuroQol-5 Dimension [EQ-5D] questionnaire, 12-Item Short Form Health Survey [SF-12], Impact on Partner and Family questionnaire, and Work Productivity and Activity Impairment [WPAI] questionnaire); satisfaction with treatment (using the PPSI and Patients' Global Impression of Change [PGIC] scale); safety evaluations (including eC-SSRS); blood collection for pharmacokinetics, immunogenicity, biomarker, and pharmacogenomics (unless not allowed per local regulation) analyses; and urine sampling for biomarker analysis will be performed at prespecified time points.

The end of study is defined as the last visit of the last patient.

### Investigational Medicinal Products Used in the Study

Investigational medicinal product is defined as the test IMP and the placebo IMP. Details of the test and placebo IMPs are presented in table below.

| IMP Name | Test IMP | Placebo IMP |
|---|---|---|
| Trade name and INN, if | TEV-48125 (formerly LBR-101, | n/a |
| applicable, or | PF-04427429, or RN307) | |
| company-assigned number | | |
| Formulation | Solution for injection | Solution for injection |
| Unit dose strength(s) | 225 mg/mL | n/a |
| Dosage level(s) | 900 mg iv loading dose followed | |
| | by 225 mg sc monthly | |
| | or | |
| | 675 mg sc quarterly | |
| Route of Administration | TEV-48125 will be administered | Placebo will be administered as |
| | as iv infusions and sc injections | iv infusions and sc injections by |
| | by qualified study personnel at | qualified study personnel at the |
| | the study center. | study center. |
| Packaging | TEV-48125 will be provided in | Placebo will be provided in |
| | prefilled syringes contained in | prefilled syringes contained in |
| | uniquely numbered kits and | uniquely numbered kits and |
| | stored (refrigerated at 2° C. to | stored (refrigerated at 2° C. to |
| | 8° C.) on site. Prefilled syringes | 8° C.) on site. Prefilled syringes |
| | will contain TEV-48125 at a | will contain the same vehicle and |
| | concentration of 150 mg/mL. | excipients as those for active |
| | | infusion and injection. |

IMP = Investigational Medicinal Product;
iv = intravenous;
n/a = not applicable;
sc = subcutaneous.

The TEV-48125 doses, regimens, and routes of administration to be evaluated in this double blind, double-dummy, placebo-controlled study were selected on the basis of three key factors. First, simulations suggest that Cₘₐₓ is the most significant pharmacokinetic parameter in the efficacy of TEV-48125 (in migraine). As CH is considered one of the most severe forms of pain a person can experience, treatments that provide quick and lasting relief (i.e., for the duration of the cluster period) are a priority for this patient population. Second, the biological nature of the disease mandates the need for any treatment to desensitize the third order neuron, not the second (as is the case in migraine), suggesting that high levels of blockade at the first neuron would be necessary. Third, the favorable safety profile of the drug and calculated safety margins based modelling and simulations, as well as clinical and nonclinical safety data on exposure, suggest that the proposed doses, regimens, and routes of administration will not present any safety concerns.

In the current study, high doses are planned for the first dose (900 mg iv or 675 mg sc) in order to provide a rapid response, especially following iv infusion where higher peak plasma concentrations (Cₘₐₓ) generally occur at or shortly after the end of infusion (median tmax values of 1.0 to 5.0 hours after starting the iv infusion) compared with 96 to 108 hours postdose for sc injections. The two forms of loading dose will provide data to confirm the benefit of either the iv or sc as loading dose. Monthly doses of TEV-48125 at 225 mg sc were added to the initial dose of 900 mg iv for maintenance of efficacy. Based on modelling, the inclusion of a loading dose should allow patients to reach steady state faster. The dose of 675 mg sc quarterly in this ECH population will allow for the evaluation of a single treatment dose taking into account the periods of remission seen with this CH form.

Administration of IMP will be via the iv and sc routes. At visit 2, IMP (test or placebo) will be administered via an approximately 1-hour iv infusion followed by 3 sc injections of IMP (test or placebo). At visits 3 and 4, IMP (test or placebo) will be administered as single sc injections. The recommended sc injection sites follow the National Institutes of Health Patient Education Guidelines of September 2015, which are available at the following website: http://www.cc.nih.gov/ccc/patient_education/pepubs/subq.pdf. The suggested sites of injection are back of upper arms, lower abdomen/belly/waistline, and front of thighs. At each visit, the injections should be given in a different location (eg, not in precisely the same place), and study staff member(s) responsible for administration of injections should inspect previous injection sites to ensure that they are free of bruising and tenderness and that proper rotation of sites is performed. The total number of IMP sc injections and their locations will be recorded for each dosing visit (visits 2 through 4, test or placebo). A 1.5-mL volume from each prefilled syringe must be injected sc for dosing to be considered complete.

### Test Investigational Medicinal Product

TEV-48125 is a humanized IgG2a/kappa monoclonal antibody derived from a murine precursor. TEV-48125 for CH is being developed for iv and sc administration.

### Starting Dose and Dose Levels

Patients randomized to receive TEV-48125 will receive either 900 mg or 675 mg. At visit 2 (week 0), iv administration of treatment (IMP or placebo) will precede sc administration (IMP or placebo). Patients randomized to the 900-mg iv loading dose group will receive 900 mg of TEV-48125 administered via an approximately 1-hour iv infusion followed by placebo as 3 sc injections at visit 2 (week 0) and TEV-48125 at 225 mg administered as single sc injections (225 mg/1.5 mL) at visits 3 and 4 (weeks 4 and 8, respectively). Patients randomized to the TEV-48125 675-mg sc quarterly group will receive placebo administered via an approximately one hour iv infusion followed by TEV-48125 at 675 mg administered as 3 sc injections (225 mg/1.5 mL) at visit 2 (week 0) and placebo administered as single sc injections at visits 3 and 4 (weeks 4 and 8, respectively).

### Study Objectives and Endpoints

Primary Objective and Endpoint:

The primary objective of this study is to demonstrate the efficacy of fremanezumab in the prevention of episodic cluster headache (ECH) in adult patients.

The primary efficacy endpoint of this study is the mean change from baseline (run-in period) in the weekly average number of cluster headache (CH) attacks during the 4-week period after administration of the first dose of the investigational medicinal product (IMP), i.e., based on week 0 to 4 data.

Secondary Objective and Endpoints:

A secondary objective of this study is to further demonstrate the efficacy of fremanezumab in the prevention of ECH in adult patients.

The secondary efficacy endpoints to further demonstrate efficacy are:
the proportion of patients with a ≥50% reduction from baseline (run-in period) in the weekly average number of CH attacks during the 4-week period after the first dose of the IMP, i.e., based on week 0 to 4 data;
the mean change from baseline (run-in period) in the number of CH attacks during the 12-week period after administration of the first dose of the IMP, i.e., based on week 0 to 12 data;
the mean change from baseline (run-in period) in the number of CH attacks during the 4-week period after administration of the third dose of the IMP, i.e., based on week 8 to 12 data;
the mean change from baseline (run-in period) in the weekly average number of days with use of cluster-specific acute headache medications (triptans and ergot compounds) during the 12-week period after administration of the first dose of the IMP, i.e., based on week 0 to 12 data;
the mean change from baseline (run-in period) in the weekly average number of days oxygen is used to treat ECH during the 12-week period after administration of the first dose of the IMP, i.e., based on week 0 to 12 data; and
assessment of patient's perceived improvement, as measured by the Patient-Perceived Satisfactory Improvement (PPSI) at 1, 4, 8, and 12 weeks after administration of the first dose of the IMP relative to baseline (day 0).

A secondary objective of this study is to evaluate the safety of fremanezumab in adult patients with ECH.

The secondary safety endpoints are as follows:
occurrence of adverse events throughout the study;
clinical laboratory (serum chemistry, hematology, coagulation, and urinalysis) test results at each visit;
vital signs (systolic and diastolic blood pressure, oral temperature, and pulse rate) measurements at each visit. Note: Oxygen saturation will be measured in cases of suspected anaphylaxis and severe hypersensitivity. Respiratory rate will also be measured in these cases but not as a standard vital sign;
12-lead electrocardiogram (ECG) findings at screening, baseline, and week 12;
use of concomitant medication during the study;
clinically significant changes in physical examinations, including body weight;
injection site reaction (i.e., erythema, induration, and ecchymosis) and injection site pain assessments;
occurrence of hypersensitivity/anaphylaxis reactions; and
suicidal ideation and behavior as measured by the electronic Columbia Suicide Severity Rating Scale (eCSSRS).

Antibody Sequences
G1 heavy chain variable region amino acid sequence
(SEQ ID NO: 1)
EVQLVESGGGLVQPGGSLRLSCAASGFTFSNYWISVVVRQAPGKGLEVVV
AEIRSESDASATHYAEAVKGRFTISRDNAKNSLYLQMNSLRAEDTAVYYC
LAYFDYGLAIQNYWGQGTLVTVSS
G1 light chain variable region amino acid sequence
(SEQ ID NO: 2)
EIVLTQSPATLSLSPGERATLSCKASKRVTTYVSVVYQQKPGQAPRLLIY
GASNRYLGIPARFSGSGSGTDFTLTISSLEPEDFAVYYCSQSYNYPYTFG
QGTKLEIK
G1 CDR H1 (extended CDR)
(SEQ ID NO: 3)
GFTFSNYWIS
G1 CDR H2 (extended CDR)
(SEQ ID NO: 4)
EIRSESDASATHYAEAVKG
G1 CDR H3
(SEQ ID NO: 5)
YFDYGLAIQNY
G1 CDR L1
(SEQ ID NO: 6)
KASKRVTTYVS
G1 CDR L2
(SEQ ID NO: 7)
GASNRYL
G1 CDR L3
(SEQ ID NO: 8)
SQSYNYPYT
G1 heavy chain variable region nucleotide sequence
(SEQ ID NO: 9)
GAAGTTCAGCTGGTTGAATCCGGTGGTGGTCTGGTTCAGCCAGGTGGTTC
CCTGCGTCTGTCCTGCGCTGCTTCCGGTTTCACCTTCTCCAACTACTGGA
TCTCCTGGGTTCGTCAGGCTCCTGGTAAAGGTCTGGAATGGGTTGCTGAA
ATCCGTTCCGAATCCGACGCGTCCGCTACCCATTACGCTGAAGCTGTTAA
AGGTCGTTTCACCATCTCCCGTGACAACGCTAAGAACTCCCTGTACCTGC
AGATGAACTCCCTGCGTGCTGAAGACACCGCTGTTTACTACTGCCTGGCT
TACTTTGACTACGGTCTGGCTATCCAGAACTACTGGGGTCAGGGTACCCT
GGTTACCGTTTCCTCC
G1 light chain variable region nucleotide sequence
(SEQ ID NO: 10)
GAAATCGTTCTGACCCAGTCCCCGGCTACCCTGTCCCTGTCCCCAGGTGA
ACGTGCTACCCTGTCCTGCAAAGCTTCCAAACGGGTTACCACCTACGTTT
CCTGGTACCAGCAGAAACCCGGTCAGGCTCCTCGTCTGCTGATCTACGGT
GCTTCCAACCGTTACCTCGGTATCCCAGCTCGTTTCTCCGGTTCCGGTTC
CGGTACCGACTTCACCCTGACCATCTCCTCCCTGGAACCCGAAGACTTCG
CTGTTTACTACTGCAGTCAGTCCTACAACTACCCCTACACCTTCGGTCAG
GGTACCAAACTGGAAATCAAA
G1 heavy chain full antibody amino acid sequence
(including modified IgG2 as described herein)
(SEQ ID NO: 11)
EVQLVESGGGLVQPGGSLRLSCAASGFTFSNYWISVVVRQAPGKGLEVVV
AEIRSESDASATHYAEAVKGRFTISRDNAKNSLYLQMNSLRAEDTAVYYC
LAYFDYGLAIQNYWGQGTLVTVSSASTKGPSVFPLAPCSRSTSESTAALG
CLVKDYFPEPVTVSWNSGALTSGVHTFPAVLQSSGLYSLSSVVTVPSSNF
GTQTYTCNVDHKPSNTKVDKTVERKCCVECPPCPAPPVAGPSVFLFPPKP
KDTLMISRTPEVTCVVVDVSHEDPEVQFNVVYVDGVEVHNAKTKPREEQF
NSTFRVVSVLTVVHQDWLNGKEYKCKVSNKGLPSSIEKTISKTKGQPREP
QVYTLPPSREEMTKNQVSLTCLVKGFYPSDIAVEWESNGQPENNYKTTPP
MLDSDGSFFLYSKLTVDKSRWQQGNVFSCSVMHEALHNHYTQKSLSLSPG
K
G1 light chain full antibody amino acid sequence
(SEQ ID NO: 12)
EIVLTQSPATLSLSPGERATLSCKASKRVTTYVSVVYQQKPGQAPRLLIY
GASNRYLGIPARFSGSGSGTDFTLTISSLEPEDFAVYYCSQSYNYPYTFG
QGTKLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC
G1 heavy chain full antibody nucleotide sequence
(including modified IgG2 as described herein)
(SEQ ID NO: 13)
GAAGTTCAGCTGGTTGAATCCGGTGGTGGTCTGGTTCAGCCAGGTGGTTC
CCTGCGTCTGTCCTGCGCTGCTTCCGGTTTCACCTTCTCCAACTACTGGA
TCTCCTGGGTTCGTCAGGCTCCTGGTAAAGGTCTGGAATGGGTTGCTGAA
ATCCGTTCCGAATCCGACGCGTCCGCTACCCATTACGCTGAAGCTGTTAA
AGGTCGTTTCACCATCTCCCGTGACAACGCTAAGAACTCCCTGTACCTGC
AGATGAACTCCCTGCGTGCTGAAGACACCGCTGTTTACTACTGCCTGGCT
TACTTTGACTACGGTCTGGCTATCCAGAACTACTGGGGTCAGGGTACCCT
GGTTACCGTTTCCTCCGCCTCCACCAAGGGCCCATCTGTCTTCCCACTGG
CCCCATGCTCCCGCAGCACCTCCGAGAGCACAGCCGCCCTGGGCTGCCTG
GTCAAGGACTACTTCCCAGAACCTGTGACCGTGTCCTGGAACTCTGGCGC
TCTGACCAGCGGCGTGCACACCTTCCCAGCTGTCCTGCAGTCCTCAGGTC
TCTACTCCCTCAGCAGCGTGGTGACCGTGCCATCCAGCAACTTCGGCACC
CAGACCTACACCTGCAACGTAGATCACAAGCCAAGCAACACCAAGGTCGA
CAAGACCGTGGAGAGAAAGTGTTGTGTGGAGTGTCCACCTTGTCCAGCCC
CTCCAGTGGCCGGACCATCCGTGTTCCTGTTCCCTCCAAAGCCAAAGGAC
ACCCTGATGATCTCCAGAACCCCAGAGGTGACCTGTGTGGTGGTGGACGT
GTCCCACGAGGACCCAGAGGTGCAGTTCAACTGGTATGTGGACGGAGTGG
AGGTGCACAACGCCAAGACCAAGCCAAGAGAGGAGCAGTTCAACTCCACC
TTCAGAGTGGTGAGCGTGCTGACCGTGGTGCACCAGGACTGGCTGAACGG
AAAGGAGTATAAGTGTAAGGTGTCCAACAAGGGACTGCCATCCAGCATCG
AGAAGACCATCTCCAAGACCAAGGGACAGCCAAGAGAGCCACAGGTGTAT
ACCCTGCCCCCATCCAGAGAGGAGATGACCAAGAACCAGGTGTCCCTGAC
CTGTCTGGTGAAGGGATTCTATCCATCCGACATCGCCGTGGAGTGGGAGT
CCAACGGACAGCCAGAGAACAACTATAAGACCACCCCTCCAATGCTGGAC
TCCGACGGATCCTTCTTCCTGTATTCCAAGCTGACCGTGGACAAGTCCAG
ATGGCAGCAGGGAAACGTGTTCTCTTGTTCCGTGATGCACGAGGCCCTGC
ACAACCACTATACCCAGAAGAGCCTGTCCCTGTCTCCAGGAAAGTAA
G1 light chain full antibody nucleotide sequence
(SEQ ID NO: 14)
GAAATCGTTCTGACCCAGTCCCCGGCTACCCTGTCCCTGTCCCCAGGTGA
ACGTGCTACCCTGTCCTGCAAAGCTTCCAAACGGGTTACCACCTACGTTT
CCTGGTACCAGCAGAAACCCGGTCAGGCTCCTCGTCTGCTGATCTACGGT
GCTTCCAACCGTTACCTCGGTATCCCAGCTCGTTTCTCCGGTTCCGGTTC
CGGTACCGACTTCACCCTGACCATCTCCTCCCTGGAACCCGAAGACTTCG
CTGTTTACTACTGCAGTCAGTCCTACAACTACCCCTACACCTTCGGTCAG
GGTACCAAACTGGAAATCAAACGCACTGTGGCTGCACCATCTGTCTTCAT
CTTCCCTCCATCTGATGAGCAGTTGAAATCCGGAACTGCCTCTGTTGTGT
GCCTGCTGAATAACTTCTATCCGCGCGAGGCCAAAGTACAGTGGAAGGTG
GATAACGCCCTCCAATCCGGTAACTCCCAGGAGAGTGTCACAGAGCAGGA
CAGCAAGGACAGCACCTACAGCCTCAGCAGCACCCTGACCCTGAGCAAAG
CAGACTACGAGAAACACAAAGTCTACGCCTGCGAAGTCACCCATCAGGGC
CTGAGTTCTCCAGTCACAAAGAGCTTCAACCGCGGTGAGTGCTAA
Amino acid sequence comparison of human and rat
CGRP (human α-CGRP (SEQ ID NO: 15); human β-CGRP
(SEQ ID NO: 43); rat α-CGRP (SEQ ID NO: 41);
and rat β-CGRP (SEQ ID NO: 44)):

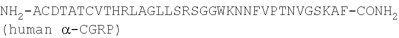

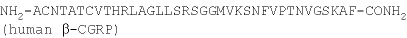

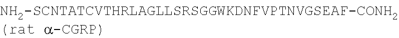

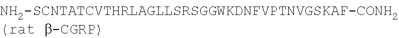

Light chain variable region LCVR17 amino acid
sequence
(SEQ ID NO: 58)
DIQMTQSPSSLSASVGDRVTITCRASQDIDNYLNVVYQQKPGKAPKLLIY
YTSEYHSGVPSRFSGSGSGTDFTFTISSLQPEDIATYYCQQGDALPPTFG
QGTKLEIK
Heavy chain variable region HCVR22 amino acid
sequence
(SEQ ID NO: 59)
QVQLVQSGAEVKKPGASVKVSCKASGYTFGNYWMQVVVRQAPGQGLEWMG
AIYEGTGDTRYIQKFAGRVTMTRDTSTSTVYMELSSLRSEDTAVYYCARL
SDYVSGFSYWGQGTLVTVSS
Light chain variable region LCVR18 amino acid
sequence
(SEQ ID NO: 60)
DIQMTQSPSSLSASVGDRVTITCRASQDIDNYLNVVYQQKPGKAPKLLIY
YTSEYHSGVPSRFSGSGSGTDFTFTISSLQPEDIATYYCQQGDALPPTFG
QGTKLEIK
Heavy chain variable region HCVR23 amino acid
sequence
(SEQ ID NO: 61)
QVQLVQSGAEVKKPGASVKVSCKASGYTFGNYWMQVVVRQAPGQGLEWMG
AIYEGTGKTVYIQKFAGRVTMTRDTSTSTVYMELSSLRSEDTAVYYCARL
SDYVSGFSYWGQGTLVTVSS
Light chain variable region LCVR19 amino acid
sequence
(SEQ ID NO: 62)
DIQMTQSPSSLSASVGDRVTITCRASKDISKYLNVVYQQKPGKAPKLLIY
YTSGYHSGVPSRFSGSGSGTDFTLTISSLQPEDFATYYCQQGDALPPTFG
GGTKVEIK
Heavy chain variable region HCVR24 amino acid
sequence
(SEQ ID NO: 63)
QVQLVQSGAEVKKPGSSVKVSCKASGYTFGNYWMQVVVRQAPGQGLEWMG
AIYEGTGKTVYIQKFADRVTITADKSTSTAYMELSSLRSEDTAVYYCARL
SDYVSGFGYWGQGTTVTVSS
Light chain variable region LCVR20 amino acid
sequence
(SEQ ID NO: 64)
DIQMTQSPSSLSASVGDRVTITCRASRPIDKYLNVVYQQKPGKAPKLLIY
YTSEYHSGVPSRFSGSGSGTDFTFTISSLQPEDIATYYCQQGDALPPTFG
QGTKLEIK
Heavy chain variable region HCVR25 amino acid
sequence
(SEQ ID NO: 65)
QVQLVQSGAEVKKPGASVKVSCKASGYTFGNYWMQVVVRQAPGQGLEWMG
AIYEGTGKTVYIQKFAGRVTMTRDTSTSTVYMELSSLRSEDTAVYYCARL
SDYVSGFGYWGQGTLVTVSS
Light chain variable region LCVR21 amino acid
sequence
(SEQ ID NO: 66)
DIQMTQSPSSLSASVGDRVTITCRASQDIDKYLNVVYQQKPGKAPKLLIY
YTSGYHSGVPSRFSGSGSGTDFTLTISSLQPEDFATYYCQQGDALPPTFG
GGTKVEIK
Heavy chain variable region HCVR26 amino acid
sequence
(SEQ ID NO: 67)
QVQLVQSGAEVKKPGSSVKVSCKASGYTFGNYWMQVVVRQAPGQGLEWMG
AIYEGTGKTVYIQKFAGRVTITADKSTSTAYMELSSLRSEDTAVYYCARL
SDYVSGFGYWGQGTTVTVSS
Light chain variable region LCVR27 amino acid
sequence
(SEQ ID NO: 68)
QVLTQSPSSLSASVGDRVTINCQASQSVYHNTYLAVVYQQKPGKVPKQLI
YDASTLASGVPSRFSGSGSGTDFTLTISSLQPEDVATYYCLGSYDCTNGD
CFVFGGGTKVEIKR
Heavy chain variable region HCVR28 amino acid
sequence
(SEQ ID NO: 69)
EVQLVESGGGLVQPGGSLRLSCAVSGIDLSGYYMNVVVRQAPGKGLEVVV
GVIGINGATYYASWAKGRFTISRDNSKTTVYLQMNSLRAEDTAVYFCARG
DIWGQGTLVTVSS
Light chain variable region LCVR29 amino acid
sequence
(SEQ ID NO: 70)
QVLTQSPSSLSASVGDRVTINCQASQSVYDNNYLAVVYQQKPGKVPKQLI
YSTSTLASGVPSRFSGSGSGTDFTLTISSLQPEDVATYYCLGSYDCSSGD
CFVFGGGTKVEIKR
Heavy chain variable region HCVR30 amino acid
sequence
(SEQ ID NO: 71)
EVQLVESGGGLVQPGGSLRLSCAVSGLDLSSYYMQVVVRQAPGKGLEVVV
GVIGINDNTYYASWAKGRFTISRDNSKTTVYLQMNSLRAEDTAVYFCARG
DIWGQGTLVTVSS
Light chain variable region LCVR31 amino acid
sequence
(SEQ ID NO: 72)
QVLTQSPSSLSASVGDRVTINCQASQSVYDNNYLAVVYQQKPGKVPKQLI
YSTSTLASGVPSRFSGSGSGTDFTLTISSLQPEDVATYYCLGSYDCSSGD
CFVFGGGTKVEIKR
Heavy chain variable region HCVR32 amino acid
sequence
(SEQ ID NO: 73)
EVQLVESGGGLVQPGGSLRLSCAVSGLDLSSYYMQVVVRQAPGKGLEVVV
GVIGINDNTYYASWAKGRFTISRDNSKTIVYLQMNSLRAEDTAVYFCARG
DIWGQGTLVTVSS
Light chain variable region LCVR33 amino acid
sequence
(SEQ ID NO: 74)
QVLTQTPSPVSAAVGSTVTINCQASQSVYHNTYLAVVYQQKPGQPPKQLI
YDASTLASGVPSRFSGSGSGTQFTLTISGVQCNDAAAYYCLGSYDCTNGD
CFVFGGGTEVVVKR
Heavy chain variable region HCVR34 amino acid
sequence
(SEQ ID NO: 75)
QSLEESGGRLVTPGTPLTLTCSVSGIDLSGYYMNVVVRQAPGKGLEWIGV
IGINGATYYASWAKGRFTISKTSSTTVDLKMTSLTTEDTATYFCARGDIW
GPGTLVTVSS
Light chain variable region LCVR35 amino acid
sequence
(SEQ ID NO: 76)
QVLTQSPSSLSASVGDRVTINCQASQSVYHNTYLAVVYQQKPGKVPKQLI
YDASTLASGVPSRFSGSGSGTDFTLTISSLQPEDVATYYCLGSYDCTNGD
CFVFGGGTKVEIKR
Heavy chain variable region HCVR36 amino acid
sequence
(SEQ ID NO: 77)
EVQLVESGGGLVQPGGSLRLSCAVSGIDLSGYYMNVVVRQAPGKGLEVVV
GVIGINGATYYASWAKGRFTISRDNSKTTVYLQMNSLRAEDTAVYFCARG
DIWGQGTLVTVSS
Light chain variable region LCVR37 amino acid
sequence
(SEQ ID NO: 78)
QSVLTQPPSVSAAPGQKVTISCSGSSSNIGNNYVSVVYQQLPGTAPKWYD
NNKRPSGIPDRFSGSKSGTSTTLGITGLQTGDEADYYCGTWDSRLSAVVF
GGGTKLTVL
Heavy chain variable region HCVR38 amino acid
sequence
(SEQ ID NO: 79)
QVQLVESGGGVVQPGRSLRLSCAASGFTFSSFGMHVVVRQAPGKGLEVVV
AVISFDGSIKYSVDSVKGRFTISRDNSKNTLFLQMNSLRAEDTAVYYCAR
DRLNYYDSSGYYHYKYYGMAVWGQGTTVTVSS

What is claimed is:

1. A method of treating a chronic cluster headache (CCH) in a subject, the method comprising:
selecting a subject who is susceptible to or having a CCH; and
administering to the subject a therapeutically effective amount of a monoclonal antibody that modulates the calcitonin gene-related peptide (CGRP) pathway, wherein the monoclonal antibody is administered intravenously at a dose of about 900 mg followed by subsequent doses of about 225 mg administered subcutaneously at one month intervals.

2. The method of claim 1, wherein the administering comprises administering the antibody to the subject from a pre-filled syringe, pre-filled syringe with a needle safety device, injection pen, or auto-injector comprising a dose of the monoclonal antibody.

3. The method of claim 1, wherein the monoclonal antibody is administered as a formulation comprising the antibody at a concentration of at least about 150 mg/mL.

4. The method of claim 1, wherein the monoclonal antibody is administered in a volume of less than 2 mL.

5. The method of claim 1, wherein the monoclonal antibody is an anti-CGRP antagonist antibody.

6. The method of claim 1, wherein the monoclonal antibody is human or humanized.

7. The method of claim 1, wherein the monoclonal antibody is a humanized anti-CGRP antagonist antibody.

8. The method of claim 1, wherein the monoclonal antibody comprises a CDR H1 as set forth in SEQ ID NO:3; a CDR H2 as set forth in SEQ ID NO:4; a CDR H3 as set forth in SEQ ID NO:5; a CDR L1 as set forth in SEQ ID NO:6; a CDR L2 as set forth in SEQ ID NO:7; and a CDR L3 as set forth in SEQ ID NO:8.

9. The method of claim 1, wherein the monoclonal antibody is an IgG1, IgG2, IgG3, or IgG4 antibody.

10. The method of claim 1, wherein the subject is human.

11. A method of treating an episodic cluster headache (ECH) in a subject, the method comprising:
selecting a subject who is susceptible to or having a ECH; and
administering to the subject a therapeutically effective amount of a monoclonal antibody that modulates the calcitonin gene-related peptide (CGRP) pathway, wherein the monoclonal antibody is administered intravenously at a dose of about 900 mg followed by subsequent doses of about 225 mg administered subcutaneously at one month intervals.

12. The method of claim 11, wherein the administering comprises administering the antibody to the subject from a pre-filled syringe, pre-filled syringe with a needle safety device, injection pen, or auto-injector comprising a dose of the monoclonal antibody.

13. The method of claim 11, wherein the monoclonal antibody is administered as a formulation comprising the antibody at a concentration of at least about 150 mg/mL.

14. The method of claim 11, wherein the monoclonal antibody is administered in a volume of less than 2 mL.

15. The method of claim 11, wherein the monoclonal antibody is an anti-CGRP antagonist antibody.

16. The method of claim 11, wherein the monoclonal antibody is human or humanized.

17. The method of claim 11, wherein the monoclonal antibody is a humanized anti-CGRP antagonist antibody.

18. The method of claim 11, wherein the monoclonal antibody comprises a CDR H1 as set forth in SEQ ID NO:3; a CDR H2 as set forth in SEQ ID NO:4; a CDR H3 as set forth in SEQ ID NO:5; a CDR L1 as set forth in SEQ ID NO:6; a CDR L2 as set forth in SEQ ID NO:7; and a CDR L3 as set forth in SEQ ID NO:8.

19. The method of claim 11, wherein the monoclonal antibody is an IgG1, IgG2, IgG3, or IgG4 antibody.

20. The method of claim 11, wherein the subject is human.
